SEQUENCE LISTING (529 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 117 aa, Artificial Sequence; identical to SEQ ID NO: 1
  Description of Artificial Sequence Synthetic polypeptide

SEQ ID NO: 4 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 5 - protein, 117 aa, Artificial Sequence; identical to SEQ ID NO: 1
  Description of Artificial Sequence Synthetic polypeptide

SEQ ID NO: 6 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 7 - protein, 117 aa, Artificial Sequence; identical to SEQ ID NO: 1
  Description of Artificial Sequence Synthetic polypeptide

SEQ ID NO: 8 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 117 aa, Artificial Sequence; identical to SEQ ID NO: 1
  Description of Artificial Sequence Synthetic polypeptide

SEQ ID NO: 10 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 11 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 12 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 13 - protein, 117 aa, Artificial Sequence; identical to SEQ ID NO: 1
  Description of Artificial Sequence Synthetic polypeptide

SEQ ID NO: 14 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 15 - protein, 117 aa, Artificial Sequence; identical to SEQ ID NO: 1
  Description of Artificial Sequence Synthetic polypeptide

SEQ ID NO: 16 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 17 - protein, 117 aa, Artificial Sequence; identical to SEQ ID NO: 1
  Description of Artificial Sequence Synthetic polypeptide

SEQ ID NO: 18 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 19 - protein, 117 aa, Artificial Sequence; identical to SEQ ID NO: 1
  Description of Artificial Sequence Synthetic polypeptide

SEQ ID NO: 20 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 21 - protein, 117 aa, Artificial Sequence; identical to SEQ ID NO: 1
  Description of Artificial Sequence Synthetic polypeptide

SEQ ID NO: 22 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 23 - protein, 117 aa, Artificial Sequence; identical to SEQ ID NO: 1
  Description of Artificial Sequence Synthetic polypeptide

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 117 aa, Artificial Sequence; identical to SEQ ID NO: 1
  Description of Artificial Sequence Synthetic polypeptide

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 27 - protein, 117 aa, Artificial Sequence; identical to SEQ ID NO: 1
  Description of Artificial Sequence Synthetic polypeptide

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 29 - protein, 117 aa, Artificial Sequence; identical to SEQ ID NO: 1
  Description of Artificial Sequence Synthetic polypeptide

SEQ ID NO: 30 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 31 - protein, 117 aa, Artificial Sequence; identical to SEQ ID NO: 1
  Description of Artificial Sequence Synthetic polypeptide

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 33 - protein, 117 aa, Artificial Sequence; identical to SEQ ID NO: 1
  Description of Artificial Sequence Synthetic polypeptide

SEQ ID NO: 34 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 35 - protein, 117 aa, Artificial Sequence; identical to SEQ ID NO: 1
  Description of Artificial Sequence Synthetic polypeptide

SEQ ID NO: 36 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 37 - protein, 117 aa, Artificial Sequence; identical to SEQ ID NO: 1
  Description of Artificial Sequence Synthetic polypeptide

SEQ ID NO: 38 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 39 - protein, 117 aa, Artificial Sequence; identical to SEQ ID NO: 1
  Description of Artificial Sequence Synthetic polypeptide

SEQ ID NO: 40 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 41 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 42 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 43 - protein, 9 aa, Artificial Sequence
  Description of Artificial Sequence Synthetic peptide
GTFSSYAIS

SEQ ID NO: 44 - protein, 17 aa, Artificial Sequence
  Description of Artificial Sequence Synthetic peptide
GIIPIFGTANYAQKFQG

SEQ ID NO: 45 - protein, 18 aa, Artificial Sequence
  Description of Artificial Sequence Synthetic peptide
ARGDSSIRHAYYYYGMDV

SEQ ID NO: 46 - protein, 17 aa, Artificial Sequence
  Description of Artificial Sequence Synthetic peptide
KSSQSVLYSSNNKNYLA

SEQ ID NO: 47 - protein, 7 aa, Artificial Sequence
  Description of Artificial Sequence Synthetic peptide
WASTRES

SEQ ID NO: 48 - protein, 9 aa, Artificial Sequence
  Description of Artificial Sequence Synthetic peptide
QQYYSTPIT

SEQ ID NO: 49 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 50 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 51 - protein, 11 aa, Artificial Sequence
  Description of Artificial Sequence Synthetic peptide
GSISSSSYYWG

SEQ ID NO: 52 - protein, 16 aa, Artificial Sequence
  Description of Artificial Sequence Synthetic peptide
SIYYSGSTYYNPSLKS

SEQ ID NO: 53 - protein, 13 aa, Artificial Sequence
  Description of Artificial Sequence Synthetic peptide
ARGSDRFHPYFDY

SEQ ID NO: 54 - protein, 11 aa, Artificial Sequence
  Description of Artificial Sequence Synthetic peptide
RASQSVSRYLA

SEQ ID NO: 55 - protein, 7 aa, Artificial Sequence
  Description of Artificial Sequence Synthetic peptide
DASNRAT

SEQ ID NO: 56 - protein, 9 aa, Artificial Sequence
  Description of Artificial Sequence Synthetic peptide
QQFDTWPPT

SEQ ID NO: 57 - protein, 117 aa, Artificial Sequence; identical to SEQ ID NO: 1
  Description of Artificial Sequence Synthetic polypeptide

SEQ ID NO: 58 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 59 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 60 - protein, 113 aa, Artificial Sequence [sequence omitted]

SEQ ID NO: 61 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 62 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 63 - protein, 9 aa, Artificial Sequence
  Description of Artificial Sequence Synthetic peptide
YTFTSYYMH

SEQ ID NO: 64 - protein, 17 aa, Artificial Sequence
  Description of Artificial Sequence Synthetic peptide
IINPSGGSTSYAQKFQG

SEQ ID NO: 65 - protein, 19 aa, Artificial Sequence
  Description of Artificial Sequence Synthetic peptide
ARGAPNYGDTTHDYYYMDV

SEQ ID NO: 66 - protein, 11 aa, Artificial Sequence
  Description of Artificial Sequence Synthetic peptide
RASQSVSSNLA

SEQ ID NO: 67 - protein, 7 aa, Artificial Sequence
  Description of Artificial Sequence Synthetic peptide
GASTRAT

SEQ ID NO: 68 - protein, 9 aa, Artificial Sequence
  Description of Artificial Sequence Synthetic peptide
QQYDDWPFT

SEQ ID NO: 69 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 70 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 71 - protein, 9 aa, Artificial Sequence
  Description of Artificial Sequence Synthetic peptide
YTFTGYYMH

SEQ ID NO: 72 - protein, 17 aa, Artificial Sequence
  Description of Artificial Sequence Synthetic peptide
WINPNSGGTNYAQKFQG

SEQ ID NO: 73 - protein, 17 aa, Artificial Sequence
  Description of Artificial Sequence Synthetic peptide
ARDTGEYYDTDDHGMDV

SEQ ID NO: 74 - protein, 11 aa, Artificial Sequence
  Description of Artificial Sequence Synthetic peptide
RASQSVSSNLA

SEQ ID NO: 75 - protein, 7 aa, Artificial Sequence
  Description of Artificial Sequence Synthetic peptide
GASTRAT

SEQ ID NO: 76 - protein, 9 aa, Artificial Sequence
  Description of Artificial Sequence Synthetic peptide
QQDDYWPPT

SEQ ID NO: 77 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 78 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 79 - protein, 9 aa, Artificial Sequence
  Description of Artificial Sequence Synthetic peptide
FTFSSYAMS

SEQ ID NO: 80 - protein, 17 aa, Artificial Sequence
  Description of Artificial Sequence Synthetic peptide
AISGSGGSTYYADSVKG

SEQ ID NO: 81 - protein, 14 aa, Artificial Sequence
  Description of Artificial Sequence Synthetic peptide
AKDGGYYDSGAGDY

SEQ ID NO: 82 - protein, 11 aa, Artificial Sequence
  Description of Artificial Sequence Synthetic peptide
RASQGIDSWLA

SEQ ID NO: 83 - protein, 7 aa, Artificial Sequence
  Description of Artificial Sequence Synthetic peptide
AASSLQS

SEQ ID NO: 84 - protein, 9 aa, Artificial Sequence
  Description of Artificial Sequence Synthetic peptide
QQGVSYPRT

SEQ ID NO: 85 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 86 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 87 - protein, 9 aa, Artificial Sequence
  Description of Artificial Sequence Synthetic peptide
FTFSSYSMN

SEQ ID NO: 88 - protein, 17 aa, Artificial Sequence
  Description of Artificial Sequence Synthetic peptide
SISSSSSYIYYADSVKG

SEQ ID NO: 89 - protein, 15 aa, Artificial Sequence
  Description of Artificial Sequence Synthetic peptide
ARGAPMGAAAGWFDP

SEQ ID NO: 90 - protein, 11 aa, Artificial Sequence
  Description of Artificial Sequence Synthetic peptide
RASQGISSWLA

SEQ ID NO: 91 - protein, 7 aa, Artificial Sequence
  Description of Artificial Sequence Synthetic peptide
AASSLQS

SEQ ID NO: 92 - protein, 9 aa, Artificial Sequence
  Description of Artificial Sequence Synthetic peptide
QQGVSFPRT

SEQ ID NO: 93 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 94 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 95 - protein, 9 aa, Artificial Sequence
  Description of Artificial Sequence Synthetic peptide
YTFTSYYMH

SEQ ID NO: 96 - protein, 17 aa, Artificial Sequence
  Description of Artificial Sequence Synthetic peptide
IINPSGGSTSYAQKFQG

SEQ ID NO: 97 - protein, 18 aa, Artificial Sequence
  Description of Artificial Sequence Synthetic peptide
AREGAGFAYGMDYYYMDV

SEQ ID NO: 98 - protein, 11 aa, Artificial Sequence
  Description of Artificial Sequence Synthetic peptide
RASQSVSSYLA

SEQ ID NO: 99 - protein, 7 aa, Artificial Sequence
  Description of Artificial Sequence Synthetic peptide
DASNRAT

SEQ ID NO: 100 - protein, 9 aa, Artificial Sequence
  Description of Artificial Sequence Synthetic peptide
QQSDNWPFT

SEQ ID NO: 101 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 102 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 103 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 104 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 105 - protein, 9 aa, Artificial Sequence
  Description of Artificial Sequence Synthetic peptide
GSFSGYYWS

SEQ ID NO: 106 - protein, 16 aa, Artificial Sequence
  Description of Artificial Sequence Synthetic peptide
EIDHSGSTNYNPSLKS

SEQ ID NO: 107 - protein, 11 aa, Artificial Sequence
  Description of Artificial Sequence Synthetic peptide
ARARGPWSFDP

SEQ ID NO: 108 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 109 - protein, 126 aa, Artificial Sequence
  Description of Artificial Sequence Synthetic polypeptide
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSA

SEQ ID NO: 110 - protein, 108 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKR

SEQ ID NO: 111 - protein, 7 aa, Artificial Sequence
  Description of Artificial Sequence Synthetic peptide
GFTFSSY

SEQ ID NO: 112 - protein, 6 aa, Artificial Sequence
  Description of Artificial Sequence Synthetic peptide
SSRSRT

SEQ ID NO: 113 - protein, 16 aa, Artificial Sequence
  Description of Artificial Sequence Synthetic peptide
DYGGQPPYYYYYGMDV

SEQ ID NO: 114 - protein, 8 aa, Artificial Sequence
  Description of Artificial Sequence Synthetic peptide
QGISSWLA

SEQ ID NO: 115 - protein, 7 aa, Artificial Sequence
  Description of Artificial Sequence Synthetic peptide
AASSLQS

SEQ ID NO: 116 - protein, 9 aa, Artificial Sequence
  Description of Artificial Sequence Synthetic peptide
QQYNSYPRT

SEQ ID NO: 117 - protein, 126 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTPGIAARRYYYYGMDVWGQGTTVTVSS

SEQ ID NO: 118 - protein, 109 aa, Artificial Sequence
  Description of Artificial Sequence Synthetic polypeptide
SSELTQDPAVSVALGQTVRITCQGDSLRKFFASWYQQKPGQAPVLVIYGKNSRPSGIPDRFSGSNSRNTASLTITGAQAEDEGDYYCNSRDSRDNHQVFGAGTKVTVLS

SEQ ID NO: 119 - protein, 7 aa, Artificial Sequence
  Description of Artificial Sequence Synthetic peptide
GFTFSSY

SEQ ID NO: 120 - protein, 6 aa, Artificial Sequence
  Description of Artificial Sequence Synthetic peptide
SAYNGN

SEQ ID NO: 121 - protein, 17 aa, Artificial Sequence
  Description of Artificial Sequence Synthetic peptide
DTPGIAARRYYYYGMDV

SEQ ID NO: 122 - protein, 8 aa, Artificial Sequence
  Description of Artificial Sequence Synthetic peptide
SLRKFFAS

SEQ ID NO: 123 - protein, 7 aa, Artificial Sequence
  Description of Artificial Sequence Synthetic peptide
GKNSRPS

SEQ ID NO: 124 - protein, 11 aa, Artificial Sequence
  Description of Artificial Sequence Synthetic peptide
NSRDSRDNHQV

SEQ ID NO: 125 - protein, 121 aa, Artificial Sequence
  Description of Artificial Sequence Synthetic polypeptide
EVQLVESGGGLVQPGGSLRLSCAASGFTSTDYYFSWVRQAPGKGLEWVGFIRTKSKGYTTEYSGSVKGRFTISRDDSKNSLYLQMNSLKTEDTAVYYCAREPITTDPRDYWGQGTLVTVSS

SEQ ID NO: 126 - protein, 112 aa, Artificial Sequence
  Description of Artificial Sequence Synthetic polypeptide
DIVMTQSPDSLAVSLGERATINCKSSQSLFNSRTRKKYLAWYQQKPGQPPKLLIYWASKRKSGVPDRFSGSGSGTDFTLTISSLQAEDVAVYYCKQSRFLRAFGQGTKLEIK

SEQ ID NO: 127 - protein, 10 aa, Artificial Sequence
  Description of Artificial Sequence Synthetic peptide
GFTSTDYYFS

SEQ ID NO: 128 - protein, 19 aa, Artificial Sequence
  Description of Artificial Sequence Synthetic peptide
FIRTKSKGYTTEYSGSVKG

SEQ ID NO: 129 - protein, 10 aa, Artificial Sequence
  Description of Artificial Sequence Synthetic peptide
EPITTDPRDY

SEQ ID NO: 130 - protein, 16 aa, Artificial Sequence
  Description of Artificial Sequence Synthetic peptide
KSSQSLFNSRTRKKYL

SEQ ID NO: 131 - protein, 7 aa, Artificial Sequence
  Description of Artificial Sequence Synthetic peptide
WASKRKS

SEQ ID NO: 132 - protein, 8 aa, Artificial Sequence
  Description of Artificial Sequence Synthetic peptide
KQSRFLRA

SEQ ID NO: 133 - protein, 352 aa, Artificial Sequence
  Description of Artificial Sequence Synthetic polypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 134 - protein, 117 aa, Artificial Sequence
  Description of Artificial Sequence Synthetic polypeptide
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSA

SEQ ID NO: 135 - protein, 107 aa, Artificial Sequence
  Description of Artificial Sequence Synthetic polypeptide
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKR

SEQ ID NO: 136 - protein, 7 aa, Artificial Sequence
  Description of Artificial Sequence Synthetic peptide
GYTFTSY

SEQ ID NO: 137 - protein, 6 aa, Artificial Sequence
  Description of Artificial Sequence Synthetic peptide
NPSTGY

SEQ ID NO: 138 - protein, 7 aa, Artificial Sequence
  Description of Artificial Sequence Synthetic peptide
GGGVFDY

SEQ ID NO: 139 - protein, 7 aa, Artificial Sequence
  Description of Artificial Sequence Synthetic peptide
SSISYMH

SEQ ID NO: 140 - protein, 7 aa, Artificial Sequence
  Description of Artificial Sequence Synthetic peptide
TTSNLAS

SEQ ID NO: 141 - protein, 9 aa, Artificial Sequence
  Description of Artificial Sequence Synthetic peptide
HQRSTYPLT

SEQ ID NO: 142 - protein, 116 aa, Artificial Sequence
  Description of Artificial Sequence Synthetic polypeptide
QLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSA

SEQ ID NO: 143 - protein, 105 aa, Artificial Sequence
  Description of Artificial Sequence Synthetic polypeptide
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKR

SEQ ID NO: 144 - protein, 7 aa, Artificial Sequence
  Description of Artificial Sequence Synthetic peptide
GYSFTRY

SEQ ID NO: 145 - protein, 6 aa, Artificial Sequence
  Description of Artificial Sequence Synthetic peptide
YPGNSD

SEQ ID NO: 146 - protein, 8 aa, Artificial Sequence
  Description of Artificial Sequence Synthetic peptide
DYGYYFDF

SEQ ID NO: 147 - protein, 7 aa, Artificial Sequence
  Description of Artificial Sequence Synthetic peptide
SSRSYMQ

SEQ ID NO: 148 - protein, 7 aa, Artificial Sequence
  Description of Artificial Sequence Synthetic peptide
DTSKLAS

SEQ ID NO: 149 - protein, 7 aa, Artificial Sequence
  Description of Artificial Sequence Synthetic peptide
HQRSSYT

SEQ ID NO: 150 - protein, 116 aa, Artificial Sequence
  Description of Artificial Sequence Synthetic polypeptide
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSA

SEQ ID NO: 151 - protein, 108 aa, Artificial Sequence
  Description of Artificial Sequence Synthetic polypeptide
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKR

SEQ ID NO: 152 - protein, 10 aa, Artificial Sequence
  Description of Artificial Sequence Synthetic peptide
GGTFSRYIIN

SEQ ID NO: 153 - protein, 17 aa, Artificial Sequence
  Description of Artificial Sequence Synthetic peptide
RIIPILGVENYAQKFQG

SEQ ID NO: 154 - protein, 6 aa, Artificial Sequence
  Description of Artificial Sequence Synthetic peptide
KDWFDY

SEQ ID NO: 155 - protein, 12 aa, Artificial Sequence
  Description of Artificial Sequence Synthetic peptide
CRASQSVSSYLA

SEQ ID NO: 156 - protein, 7 aa, Artificial Sequence
  Description of Artificial Sequence Synthetic peptide
GASSRAT

SEQ ID NO: 157 - protein, 9 aa, Artificial Sequence
  Description of Artificial Sequence Synthetic peptide
QQYGSSPLT

SEQ ID NO: 158 - protein, 272 aa, Artificial Sequence
  Description of Artificial Sequence Synthetic polypeptide
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 159 - protein, 1032 aa, Artificial Sequence [sequence omitted]

SEQ ID NO: 160 - protein, 798 aa, Artificial Sequence
  Description of Artificial Sequence Synthetic polypeptide
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 161 - protein, 742 aa, Artificial Sequence
  Description of Artificial Sequence Synthetic polypeptide
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 162 - protein, 967 aa, Artificial Sequence
  Description of Artificial Sequence Synthetic polypeptide
MAKGFYISKSLGILGILLGVAAVCTIIALSVVYSQEKNKNANSSPVASTTPSASATTNPASATTLDQSKAWNRYRLPNTLKPDSYRVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 163 - protein, 323 aa, Artificial Sequence
  Description of Artificial Sequence Synthetic polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 164 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 165 - protein, 236 aa, Artificial Sequence
  Description of Artificial Sequence Synthetic polypeptide
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 166 - protein, 120 aa, Artificial Sequence
  Description of Artificial Sequence Synthetic polypeptide
VKLQQSGAELVKPGASVKLFCTASGFNIKDTYMHWVKQRPQQGLEWIGRIDPASGDTKYDPKFQVKATITADTSSNTAWLQLSSLTSEDTAVYYCADGMWVSTGYALDFWGQGTTVTVSS

SEQ ID NO: 167 - protein, 106 aa, Artificial Sequence
  Description of Artificial Sequence Synthetic polypeptide
SIVMTQTPKFLLVSAGDRVTITCKASQSVTNDVAWYQQKPGQSPKLLIYYASNRYTGVPDRFTGSGYGTDFTFTISTVQAEDLAVYFCQQDYSSPYTFGGGTKLEI

SEQ ID NO: 168 - protein, 7 aa, Artificial Sequence
  Description of Artificial Sequence Synthetic peptide
GFNIKDT

SEQ ID NO: 169 - protein, 6 aa, Artificial Sequence
  Description of Artificial Sequence Synthetic peptide
DPASGD

SEQ ID NO: 170 - protein, 12 aa, Artificial Sequence
  Description of Artificial Sequence Synthetic peptide
GMWVSTGYALDF

SEQ ID NO: 171 - protein, 8 aa, Artificial Sequence
  Description of Artificial Sequence Synthetic peptide
QSVTNDVA

SEQ ID NO: 172 - protein, 7 aa, Artificial Sequence
  Description of Artificial Sequence Synthetic peptide
YASNRYT

SEQ ID NO: 173 - protein, 9 aa, Artificial Sequence
  Description of Artificial Sequence Synthetic peptide
QQDYSSPYT

SEQ ID NO: 174 - protein, 115 aa, Artificial Sequence
  Description of Artificial Sequence Synthetic polypeptide
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSA

SEQ ID NO: 175 - protein, 107 aa, Artificial Sequence
  Description of Artificial Sequence Synthetic polypeptide
EIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKR

SEQ ID NO: 176 - protein, 7 aa, Artificial Sequence
  Description of Artificial Sequence Synthetic peptide
GFTFSSY

SEQ ID NO: 177 - protein, 6 aa, Artificial Sequence
  Description of Artificial Sequence Synthetic peptide
SSGGSY

SEQ ID NO: 178 - protein, 5 aa, Artificial Sequence
  Description of Artificial Sequence Synthetic peptide
QGLDY

SEQ ID NO: 179 - protein, 7 aa, Artificial Sequence
  Description of Artificial Sequence Synthetic peptide
SSINYIY

SEQ ID NO: 180 - protein, 7 aa, Artificial Sequence
  Description of Artificial Sequence Synthetic peptide
LTSNLAS

SEQ ID NO: 181 - protein, 9 aa, Artificial Sequence
  Description of Artificial Sequence Synthetic peptide
LQWSSNPLT

SEQ ID NO: 182 - protein, 117 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 183 - protein, 112 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 184 - protein, 10 aa, Artificial Sequence
  Description of Artificial Sequence Synthetic peptide
GYTFTNYNMH

SEQ ID NO: 185 - protein, 17 aa, Artificial Sequence
  Description of Artificial Sequence Synthetic peptide
TIYPGNDDTSYNQKFKD

SEQ ID NO: 186 - protein, 8 aa, Artificial Sequence
  Description of Artificial Sequence Synthetic peptide
GGYRAMDY

SEQ ID NO: 187 - protein, 16 aa, Artificial Sequence
  Description of Artificial Sequence Synthetic peptide
RSSQSIVYSNGNTYLG

SEQ ID NO: 188 - protein, 7 aa, Artificial Sequence
  Description of Artificial Sequence Synthetic peptide
KVSNRFS

SEQ ID NO: 189 - protein, 9 aa, Artificial Sequence
  Description of Artificial Sequence Synthetic peptide
FQGSHVPYT

SEQ ID NO: 190 - protein, 321 aa, Artificial Sequence
  Description of Artificial Sequence Synthetic polypeptide
MEEGQYSEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 191 - protein, 277 aa, Artificial Sequence
  Description of Artificial Sequence Synthetic polypeptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 192 - protein, 193 aa, Artificial Sequence
  Description of Artificial Sequence Synthetic polypeptide
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 193 - protein, 226 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 194 - protein, 229 aa, Artificial Sequence
  Description of Artificial Sequence Synthetic polypeptide
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 195 - protein, 288 aa, Artificial Sequence
  Description of Artificial Sequence Synthetic polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 196 - protein, 371 aa, Artificial Sequence
  Description of Artificial Sequence Synthetic polypeptide
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 197 - protein, 118 aa, Artificial Sequence
  Description of Artificial Sequence Synthetic polypeptide
EVQLVESGGGLAKPGGSLRLSCAASGFRFTFNNYYMDWVRQAPGQGLEWVSRISSSGDPTWYADSVKGRFTISRENANNTLFLQMNSLRAEDTAVYYCASLTTGSDSWGQGVLVTVSS

SEQ ID NO: 198 - protein, 107 aa, Artificial Sequence
  Description of Artificial Sequence Synthetic polypeptide
DIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIK

SEQ ID NO: 199 - protein, 9 aa, Artificial Sequence
  Description of Artificial Sequence Synthetic peptide
GFRFTFNNY

SEQ ID NO: 200 - protein, 6 aa, Artificial Sequence
  Description of Artificial Sequence Synthetic peptide
SSSGDP

SEQ ID NO: 201 - protein, 7 aa, Artificial Sequence
  Description of Artificial Sequence Synthetic peptide
LTTGSDS

SEQ ID NO: 202 - protein, 8 aa, Artificial Sequence
  Description of Artificial Sequence Synthetic peptide
QDIRYYLN

SEQ ID NO: 203 - protein, 7 aa, Artificial Sequence
  Description of Artificial Sequence Synthetic peptide
VASSLQS

SEQ ID NO: 204 - protein, 9 aa, Artificial Sequence
  Description of Artificial Sequence Synthetic peptide
LQVYSTPRT

SEQ ID NO: 205 - protein, 115 aa, Artificial Sequence
  Description of Artificial Sequence Synthetic polypeptide
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSA

SEQ ID NO: 206 - protein, 113 aa, Artificial Sequence
  Description of Artificial Sequence Synthetic polypeptide
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

SEQ ID NO: 207 - protein, 7 aa, Artificial Sequence
  Description of Artificial Sequence Synthetic peptide
GYSFTGY

SEQ ID NO: 208 - protein, 6 aa, Artificial Sequence
  Description of Artificial Sequence Synthetic peptide
IPNAGG

SEQ ID NO: 209 - protein, 5 aa, Artificial Sequence
  Description of Artificial Sequence Synthetic peptide
EGIYW

SEQ ID NO: 210 - protein, 13 aa, Artificial Sequence
  Description of Artificial Sequence Synthetic peptide
QSLVHSNGNTFLH

SEQ ID NO: 211 - protein, 7 aa, Artificial Sequence
  Description of Artificial Sequence Synthetic peptide
TVSNRFS

SEQ ID NO: 212 - protein, 9 aa, Artificial Sequence
  Description of Artificial Sequence Synthetic peptide
SQTTHVPWT

SEQ ID NO: 213 - protein, 127 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSA

SEQ ID NO: 214 - protein, 108 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKR

SEQ ID NO: 215 - protein, 7 aa, Artificial Sequence
  Description of Artificial Sequence Synthetic peptide
GYTFTGY

SEQ ID NO: 216 - protein, 6 aa, Artificial Sequence
  Description of Artificial Sequence Synthetic peptide
NPDSGG

SEQ ID NO: 217 - protein, 17 aa, Artificial Sequence
  Description of Artificial Sequence Synthetic peptide
DQPLGYCTNGVCSYFDY

SEQ ID NO: 218 - protein, 8 aa, Artificial Sequence
  Description of Artificial Sequence Synthetic peptide
QGIYSWLA

SEQ ID NO: 219 - protein, 7 aa, Artificial Sequence
  Description of Artificial Sequence Synthetic peptide
TASTLQS

SEQ ID NO: 220 - protein, 9 aa, Artificial Sequence
  Description of Artificial Sequence Synthetic peptide
QQANIFPLT

SEQ ID NO: 221 - protein, 121 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSA

SEQ ID NO: 222 - protein, 113 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRR

SEQ ID NO: 223 - protein, 7 aa, Artificial Sequence
  Description of Artificial Sequence Synthetic peptide
GFTFSSY

SEQ ID NO: 224 - protein, 6 aa, Artificial Sequence
  Description of Artificial Sequence Synthetic peptide
SYEESN

SEQ ID NO: 225 - protein, 11 aa, Artificial Sequence
  Description of Artificial Sequence Synthetic peptide
DGGIAAPGPDY

SEQ ID NO: 226 - protein, 13 aa, Artificial Sequence
  Description of Artificial Sequence Synthetic peptide
QSLLYSNGYNYLD

SEQ ID NO: 227 - protein, 7 aa, Artificial Sequence
  Description of Artificial Sequence Synthetic peptide
LGSNRAS

SEQ ID NO: 228 - protein, 9 aa, Artificial Sequence
  Description of Artificial Sequence Synthetic peptide
MQARQTPFT

SEQ ID NO: 229 - protein, 128 aa, Artificial Sequence
  Description of Artificial Sequence Synthetic polypeptide
QVQLQQSGPGLVKPSQTLSLTCAISGDSVSSNSATWNWIRQSPSRDLEWLGRTYYRSKWYRDYVGSVKSRIIINPDTSNNQFSLQLNSVTPEDTAIYYCTRAQWLGGDYPYYYSMDVWGQGTTVTVSS

SEQ ID NO: 230 - protein, 104 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIK

SEQ ID NO: 231 - protein, 9 aa, Artificial Sequence
  Description of Artificial Sequence Synthetic peptide
GDSVSSNSA

SEQ ID NO: 232 - protein, 7 aa, Artificial Sequence
  Description of Artificial Sequence Synthetic peptide
YYRSKWY

SEQ ID NO: 233 - protein, 16 aa, Artificial Sequence
  Description of Artificial Sequence Synthetic peptide
AQWLGGDYPYYYSMDV

SEQ ID NO: 234 - protein, 8 aa, Artificial Sequence
  Description of Artificial Sequence Synthetic peptide
QSVSSYLA

SEQ ID NO: 235 - protein, 7 aa, Artificial Sequence
  Description of Artificial Sequence Synthetic peptide
DASNRAT

SEQ ID NO: 236 - protein, 6 aa, Artificial Sequence
  Description of Artificial Sequence Synthetic peptide
QQRSNT

SEQ ID NO: 237 - protein, 119 aa, Artificial Sequence
  Description of Artificial Sequence Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSA

SEQ ID NO: 238 - protein, 112 aa, Artificial Sequence
  Description of Artificial Sequence Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKR

SEQ ID NO: 239 - protein, 7 aa, Artificial Sequence
  Description of Artificial Sequence Synthetic peptide
GYTFTNY

SEQ ID NO: 240 - protein, 6 aa, Artificial Sequence
  Description of Artificial Sequence Synthetic peptide
NTYTGE

SEQ ID NO: 241 - protein, 9 aa, Artificial Sequence
  Description of Artificial Sequence Synthetic peptide
DYGDYGMDY

SEQ ID NO: 242 - protein, 12 aa, Artificial Sequence
  Description of Artificial Sequence Synthetic peptide
KSVSTSGYSFMH

SEQ ID NO: 243 - protein, 7 aa, Artificial Sequence
  Description of Artificial Sequence Synthetic peptide
LASNLES

SEQ ID NO: 244 - protein, 9 aa, Artificial Sequence
  Description of Artificial Sequence Synthetic peptide
QHSREVPWT

SEQ ID NO: 245 - protein, 118 aa, Artificial Sequence
  Description of Artificial Sequence Synthetic polypeptide
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSA

SEQ ID NO: 246 - protein, 112 aa, Artificial Sequence
  Description of Artificial Sequence Synthetic polypeptide
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 247 - protein, 7 aa, Artificial Sequence
  Description of Artificial Sequence Synthetic peptide
GYTFSSY

SEQ ID NO: 248 - protein, 6 aa, Artificial Sequence
  Description of Artificial Sequence Synthetic peptide
LPGGGD

SEQ ID NO: 249 - protein, 8 aa, Artificial Sequence
  Description of Artificial Sequence Synthetic peptide
RVPIRLDY

SEQ ID NO: 250 - protein, 12 aa, Artificial Sequence
  Description of Artificial Sequence Synthetic peptide
QSVDYEGDSFLN

SEQ ID NO: 251 - protein, 7 aa, Artificial Sequence
  Description of Artificial Sequence Synthetic peptide
AASNLES

SEQ ID NO: 252 - protein, 9 aa, Artificial Sequence
  Description of Artificial Sequence Synthetic peptide
QQSNEDPLT

SEQ ID NO: 253 - protein, 128 aa, Artificial Sequence
  Description of Artificial Sequence Synthetic polypeptide
QVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSA

SEQ ID NO: 254 - protein, 111 aa, Artificial Sequence
  Description of Artificial Sequence Synthetic polypeptide
ESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLG

SEQ ID NO: 255 - protein, 8 aa, Artificial Sequence
  Description of Artificial Sequence Synthetic peptide
GGSISGGY

SEQ ID NO: 256 - protein, 6 aa, Artificial Sequence
  Description of Artificial Sequence Synthetic peptide
YSSSGN

SEQ ID NO: 257 - protein, 17 aa, Artificial Sequence
  Description of Artificial Sequence Synthetic peptide
DRLFSVVGMVYNNWFDV

SEQ ID NO: 258 - protein, 10 aa, Artificial Sequence
  Description of Artificial Sequence Synthetic peptide
TSNIGGYDLH

SEQ ID NO: 259 - protein, 7 aa, Artificial Sequence
  Description of Artificial Sequence Synthetic peptide
DINKRPS

SEQ ID NO: 260 - protein, 11 aa, Artificial Sequence
  Description of Artificial Sequence Synthetic peptide
QSYDSSLNAQV

SEQ ID NO: 261 - protein, 118 aa, Artificial Sequence
  Description of Artificial Sequence Synthetic polypeptide
EVQLLESGGGLVQPGGSLRLSCAASGFTFRSSAMHWVRQAPGKGLKWVSSVSGSGAGTYYADSVKGRFTISRDNPKNTLYLQMNSLRAEDTAVYYCVKEGGSRGFDYWGQGTLVTVSS

SEQ ID NO: 262 - protein, 108 aa, Artificial Sequence
  Description of Artificial Sequence Synthetic polypeptide
DIQMTQSPSSLSASVGDRVTITCRASQDISNYLAWFQQKPGKAPKSLIYTASSLQSGVPSKFSGSGSGTDFTLTISSLQPEDFATYYCQQYNLYPPTFGQGTKVEIKR

SEQ ID NO: 263 - protein, 7 aa, Artificial Sequence
  Description of Artificial Sequence Synthetic peptide
GFTFRSS

SEQ ID NO: 264 - protein, 17 aa, Artificial Sequence
  Description of Artificial Sequence Synthetic peptide
SVSGSGAGTYYADSVKG

SEQ ID NO: 265 - protein, 9 aa, Artificial Sequence
  Description of Artificial Sequence Synthetic peptide
EGGSRGFDY

SEQ ID NO: 266 - protein, 8 aa, Artificial Sequence
  Description of Artificial Sequence Synthetic peptide
QDISNYLA

SEQ ID NO: 267 - protein, 15 aa, Artificial Sequence
  Description of Artificial Sequence Synthetic peptide
YTASSLQSGVPSKFS

SEQ ID NO: 268 - protein, 9 aa, Artificial Sequence
  Description of Artificial Sequence Synthetic peptide
QQYNLYPPT

SEQ ID NO: 269 - protein, 335 aa, Artificial Sequence
  Description of Artificial Sequence Synthetic polypeptide
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 270 - protein, 300 aa, Artificial Sequence
  Description of Artificial Sequence Synthetic polypeptide
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 271 - protein, 310 aa, Artificial Sequence
  Description of Artificial Sequence Synthetic polypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 272 - protein, 120 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSA

SEQ ID NO: 273 - protein, 108 aa, Artificial Sequence
  Description of Artificial Sequence Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 274 - protein, 7 aa, Artificial Sequence
  Description of Artificial Sequence Synthetic peptide
GFDFSRY

SEQ ID NO: 275 - protein, 6 aa, Artificial Sequence
  Description of Artificial Sequence Synthetic peptide
NPDSST

SEQ ID NO: 276 - protein, 10 aa, Artificial Sequence
  Description of Artificial Sequence Synthetic peptide
PDGNYWYFDV

SEQ ID NO: 277 - protein, 8 aa, Artificial Sequence
  Description of Artificial Sequence Synthetic peptide
QDVGIAVA

SEQ ID NO: 278 - protein, 7 aa, Artificial Sequence
  Description of Artificial Sequence Synthetic peptide
WASTRHT

SEQ ID NO: 279 - protein, 9 aa, Artificial Sequence
  Description of Artificial Sequence Synthetic peptide
QQYSSYPYT

SEQ ID NO: 280 - protein, 118 aa, Artificial Sequence
  Description of Artificial Sequence Synthetic polypeptide
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSSA

SEQ ID NO: 281 - protein, 114 aa, Artificial Sequence
  Description of Artificial Sequence Synthetic polypeptide
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIKR

SEQ ID NO: 282 - protein, 10 aa, Artificial Sequence
  Description of Artificial Sequence Synthetic peptide
GFTFSDYYMA

SEQ ID NO: 283 - protein, 26 aa, Artificial Sequence
  Description of Artificial Sequence Synthetic peptide
SINYDGSSTYYVDSVKGRFTISRDNA

SEQ ID NO: 284 - protein, 8 aa, Artificial Sequence
  Description of Artificial Sequence Synthetic peptide
DRGYYFDY

SEQ ID NO: 285 - protein, 17 aa, Artificial Sequence
  Description of Artificial Sequence Synthetic peptide
CRSSQSLVHSNGNTYLH

SEQ ID NO: 286 - protein, 7 aa, Artificial Sequence
  Description of Artificial Sequence Synthetic peptide
KVSNRFS

SEQ ID NO: 287 - protein, 10 aa, Artificial Sequence
  Description of Artificial Sequence Synthetic peptide
SQSTHVPPFT

SEQ ID NO: 288 - protein, 123 aa, Artificial Sequence
  Description of Artificial Sequence Synthetic polypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSA

SEQ ID NO: 289 - protein, 108 aa, Artificial Sequence
  Description of Artificial Sequence Synthetic polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKR

SEQ ID NO: 290 - protein, 7 aa, Artificial Sequence
  Description of Artificial Sequence Synthetic peptide
GYTFSNY

SEQ ID NO: 291 - protein, 6 aa, Artificial Sequence
  Description of Artificial Sequence Synthetic peptide
LPGTGR

SEQ ID NO: 292 - protein, 13 aa, Artificial Sequence
  Description of Artificial Sequence Synthetic peptide
RDYYGNFYYAMDY

SEQ ID NO: 293 - protein, 8 aa, Artificial Sequence
  Description of Artificial Sequence Synthetic peptide
QGINNYLN

SEQ ID NO: 294 - protein, 7 aa, Artificial Sequence
  Description of Artificial Sequence Synthetic peptide
YTSTLQS

SEQ ID NO: 295 - protein, 9 aa, Artificial Sequence
  Description of Artificial Sequence Synthetic peptide
QQYSKLPRT

SEQ ID NO: 296 - protein, 123 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSA

SEQ ID NO: 297 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 298 - protein, 7 aa, Artificial Sequence
  Description of Artificial Sequence Synthetic peptide
GFTFNSF

SEQ ID NO: 299 - protein, 6 aa, Artificial Sequence
  Description of Artificial Sequence Synthetic peptide
SGSGGG

SEQ ID NO: 300 - protein, 13 aa, Artificial Sequence
  Description of Artificial Sequence Synthetic peptide
DKILWFGEPVFDY

SEQ ID NO: 301 - protein, 8 aa, Artificial Sequence
  Description of Artificial Sequence Synthetic peptide
QSVSSYLA

SEQ ID NO: 302 - protein, 7 aa, Artificial Sequence
  Description of Artificial Sequence Synthetic peptide
DASNRAT

SEQ ID NO: 303 - protein, 9 aa, Artificial Sequence
  Description of Artificial Sequence Synthetic peptide
QQRSNWPPT

SEQ ID NO: 304 - protein, 120 aa, Artificial Sequence
  Description of Artificial Sequence Synthetic polypeptide
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 305 - protein, 108 aa, Artificial Sequence
  Description of Artificial Sequence Synthetic polypeptide
DIELTQPPSVSVAPGQTARISCSGDNLRHYYWWYQQKPGQAPVLVIYGDSKRPSGIPERFSGSNSGNTATLTISGTQAEDEADYYCQTYTGGASLVFGGGTKLTVLGQ

SEQ ID NO: 306 - protein, 10 aa, Artificial Sequence
  Description of Artificial Sequence Synthetic peptide
GFTFSSYYMN

SEQ ID NO: 307 - protein, 26 aa, Artificial Sequence
  Description of Artificial Sequence Synthetic peptide
GISGDPSNTYYADSVKGRFTISRDNS

SEQ ID NO: 308 - protein, 11 aa, Artificial Sequence
  Description of Artificial Sequence Synthetic peptide
DLPLVYTGFAY

SEQ ID NO: 309 - protein, 10 aa, Artificial Sequence
  Description of Artificial Sequence Synthetic peptide
SGDNLRHYYW

SEQ ID NO: 310 - protein, 7 aa, Artificial Sequence
  Description of Artificial Sequence Synthetic peptide
GDSKRPS

SEQ ID NO: 311 - protein, 10 aa, Artificial Sequence
  Description of Artificial Sequence Synthetic peptide
QTYTGGASLV

SEQ ID NO: 312 - protein, 177 aa, Artificial Sequence
  Description of Artificial Sequence Synthetic polypeptide
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 313 - protein, 178 aa, Artificial Sequence
  Description of Artificial Sequence Synthetic polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 314 - protein, 598 aa, Artificial Sequence
  Description of Artificial Sequence Synthetic polypeptide
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 315 - protein, 650 aa, Artificial Sequence
  Description of Artificial Sequence Synthetic polypeptide
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 316 - protein, 631 aa, Artificial Sequence
  Description of Artificial Sequence Synthetic polypeptide
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 317 - protein, 448 aa, Artificial Sequence
  Description of Artificial Sequence Synthetic polypeptide
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 318 - protein, 590 aa, Artificial Sequence
  Description of Artificial Sequence Synthetic polypeptide
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 319 - protein, 489 aa, Artificial Sequence
  Description of Artificial Sequence Synthetic polypeptide
MTPIVTVLICLRLSLGPRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVENLIRMGIAGLVLVVLGILLFEAQHSQRSL

SEQ ID NO: 320 - protein, 483 aa, Artificial Sequence
  Description of Artificial Sequence Synthetic polypeptide
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSEAAETLSPSQNKTDSTTTSLGQHPQDYTVENLIRMGVAGLVLVVLGILLFEAQHSQRSLQDAAGR

SEQ ID NO: 321 - protein, 439 aa, Artificial Sequence
  Description of Artificial Sequence Synthetic polypeptide
MTPILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSDSKAGE

SEQ ID NO: 322 - protein, 499 aa, Artificial Sequence
  Description of Artificial Sequence Synthetic polypeptide
MTLILTSLLFFGLSLGPRTRVQAENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVEPWEQI

SEQ ID NO: 323 - protein, 299 aa, Artificial Sequence
  Description of Artificial Sequence Synthetic polypeptide
MAPWSHPSAQLQPVGGDAVSPALMVLLCLGLSLGPRTHVQAGNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIRMGMAGLILVVLGILIFQDWHSQRSPQAAAGR

SEQ ID NO: 324 - protein, 481 aa, Artificial Sequence
  Description of Artificial Sequence Synthetic polypeptide
MTPALTALLCLGLSLGPRTRVQAGPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSGHSGGSSLPPTGPPSTPASHAKDYTVENLIRMGMAGLVLVFLGILLFEAQHSQRNPQDAAGR

SEQ ID NO: 325 - protein, 125 aa, Artificial Sequence
  Description of Artificial Sequence Synthetic polypeptide
MDVQLQESGGGSVQAGGSLRLSCPASGYTFSHYCMGWNRQAPGKEREEVATIDTDDTPTYADSVMGRFTISRDNANNALYLQMNDLKPEDTSMYYCAIWMKLRGSCHDRRLEVRGQGTQVTVSIN

SEQ ID NO: 326 - protein, 9 aa, Artificial Sequence
  Description of Artificial Sequence Synthetic peptide
GYTFSHYCM

SEQ ID NO: 327 - protein, 9 aa, Artificial Sequence
  Description of Artificial Sequence Synthetic peptide
TIDTDDTPT

SEQ ID NO: 328 - protein, 16 aa, Artificial Sequence
  Description of Artificial Sequence Synthetic peptide
AIWMKLRGSCHDRRLE

SEQ ID NO: 329 - protein, 127 aa, Artificial Sequence
  Description of Artificial Sequence Synthetic polypeptide
MDVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSIT

SEQ ID NO: 330 - protein, 9 aa, Artificial Sequence
  Description of Artificial Sequence Synthetic peptide
GYTHSSYCM

SEQ ID NO: 331 - protein, 9 aa, Artificial Sequence
  Description of Artificial Sequence Synthetic peptide
SIDSDGTTS

SEQ ID NO: 332 - protein, 19 aa, Artificial Sequence
  Description of Artificial Sequence Synthetic peptide
AARFGPMGCVDLSTLSFGH

SEQ ID NO: 333 - protein, 118 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 334 - protein, 108 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 335 - protein, 7 aa, Artificial Sequence
  Description of Artificial Sequence Synthetic peptide
GFTFSSY

SEQ ID NO: 336 - protein, 6 aa, Artificial Sequence
  Description of Artificial Sequence Synthetic peptide
SYDGNN

SEQ ID NO: 337 - protein, 9 aa, Artificial Sequence
  Description of Artificial Sequence Synthetic peptide
TGWLGPFDY

SEQ ID NO: 338 - protein, 9 aa, Artificial Sequence
  Description of Artificial Sequence Synthetic peptide
QSVGSSYLA

SEQ ID NO: 339 - protein, 7 aa, Artificial Sequence
  Description of Artificial Sequence Synthetic peptide
GAFSRAT

SEQ ID NO: 340 - protein, 9 aa, Artificial Sequence
  Description of Artificial Sequence Synthetic peptide
QQYGSSPWT

SEQ ID NO: 341 - protein, 451 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 342 - protein, 214 aa, Artificial Sequence
  Description of Artificial Sequence Synthetic polypeptide
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 7 aa, Artificial Sequence
  Description of Artificial Sequence Synthetic peptide
GFTFSSY

SEQ ID NO: 344 - protein, 6 aa, Artificial Sequence
  Description of Artificial Sequence Synthetic peptide
WYDGSN

SEQ ID NO: 345 - protein, 16 aa, Artificial Sequence
  Description of Artificial Sequence Synthetic peptide
DPRGATLYYYYYGMDV

SEQ ID NO: 346 - protein, 8 aa, Artificial Sequence
  Description of Artificial Sequence Synthetic peptide
QSINSYLD

SEQ ID NO: 347 - protein, 7 aa, Artificial Sequence
  Description of Artificial Sequence Synthetic peptide
AASSLQS

SEQ ID NO: 348 - protein, 9 aa, Artificial Sequence
  Description of Artificial Sequence Synthetic peptide
QQYYSTPFT

SEQ ID NO: 349 - protein, 118 aa, Artificial Sequence
  Description of Artificial Sequence Synthetic polypeptide
EVQLVESGGGSVQAGESLRLSCAASGSIFSSNAMAWYRQAPGKQRDLVAGINSVGITKYADSVKGRFTISRDNAKNTVYLQMNSLKPEDTAVYYCTSDPRRGWDTRYWGQGTQVTVSS

SEQ ID NO: 350 - protein, 10 aa, Artificial Sequence
  Description of Artificial Sequence Synthetic peptide
GSIFSSNAMA

SEQ ID NO: 351 - protein, 9 aa, Artificial Sequence
  Description of Artificial Sequence Synthetic peptide
AINSVGVTK

SEQ ID NO: 352 - protein, 10 aa, Artificial Sequence
  Description of Artificial Sequence Synthetic peptide
DPRRGWDTRY

SEQ ID NO: 353 - protein, 117 aa, Artificial Sequence
  Description of Artificial Sequence Synthetic polypeptide
VQLVESGGGLVQPGGSLRLSCAASGSIFSSTAMAWYRQAPGKRRDLVAAISSVGVTKYADSVKGRFTISRDNSKNTVYLQMNSLRPEDTAVYYCTSDPRRGWDTRYWGQGTLVTVSS

SEQ ID NO: 354 - protein, 10 aa, Artificial Sequence
  Description of Artificial Sequence Synthetic peptide
GSIFSSTAMA

SEQ ID NO: 355 - protein, 9 aa, Artificial Sequence
  Description of Artificial Sequence Synthetic peptide
QNDYSYPYT

SEQ ID NO: 356 - protein, 9 aa, Artificial Sequence
  Description of Artificial Sequence Synthetic peptide
AISSVGVTK

SEQ ID NO: 357 - protein, 10 aa, Artificial Sequence
  Description of Artificial Sequence Synthetic peptide
DPRRGWDTRY

SEQ ID NO: 358 - protein, 121 aa, Artificial Sequence
  Description of Artificial Sequence Synthetic polypeptide
EVQLVESGGDLVKPGGSLKLSCAAFGFTFSRYGMSWVRQTPDKRLEWVATITSGGIYTYYPDSVKGRFTISRDNAKNTLYLQMSSLKSEETAMYYCARHGQFGDYYGMDYWGQGTSVTVSS

SEQ ID NO: 359 - protein, 112 aa, Artificial Sequence
  Description of Artificial Sequence Synthetic polypeptide
DVVMTQTPLSLPVSLGDQASISCRSSQSLLHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 360 - protein, 10 aa, Artificial Sequence
  Description of Artificial Sequence Synthetic peptide
GFTFSRYGMS

SEQ ID NO: 361 - protein, 17 aa, Artificial Sequence
  Description of Artificial Sequence Synthetic peptide
TITSGGIYTYYPDSVKG

SEQ ID NO: 362 - protein, 12 aa, Artificial Sequence
  Description of Artificial Sequence Synthetic peptide
HGQFGDYYGMDY

SEQ ID NO: 363 - protein, 16 aa, Artificial Sequence
  Description of Artificial Sequence Synthetic peptide
RSSQSLLHSNGNTYLH

SEQ ID NO: 364 - protein, 7 aa, Artificial Sequence
  Description of Artificial Sequence Synthetic peptide
KVSNRFS

SEQ ID NO: 365 - protein, 9 aa, Artificial Sequence
  Description of Artificial Sequence Synthetic peptide
SQSTHVPYT

SEQ ID NO: 366 - protein, 123 aa, Artificial Sequence
  Description of Artificial Sequence Synthetic polypeptide
QVQLVQSGSELKKPGASVKVSCKASGYTFTHYGMNWVRQAPGQGLKWMGWINTYTGEPTYADDFKGRFVFSLDTSVSTAYLQISSLKAEDTAVYYCARRRYEGNYVFYYFDYWGQGTTVTVSS

SEQ ID NO: 367 - protein, 107 aa, Artificial Sequence
  Description of Artificial Sequence Synthetic polypeptide
DIQMTQSPSSLSASVGDRVTITCRASENIYSYFSWYQQKPGKAPKLLIYTAKTLAEGVPSRFSGSGSGTDFTLTISSLQPEDFATYYCQHHYVTPYTFGGGTKVEIK

SEQ ID NO: 368 - protein, 8 aa, Artificial Sequence
  Description of Artificial Sequence Synthetic peptide
GYTFTHYG

SEQ ID NO: 369 - protein, 7 aa, Artificial Sequence
  Description of Artificial Sequence Synthetic peptide
NTYTGEP

SEQ ID NO: 370 - protein, 16 aa, Artificial Sequence
  Description of Artificial Sequence Synthetic peptide
ARRRYEGNYVFYYFDY

SEQ ID NO: 371 - protein, 11 aa, Artificial Sequence
  Description of Artificial Sequence Synthetic peptide
RASENIYSYFS

SEQ ID NO: 372 - protein, 7 aa, Artificial Sequence
  Description of Artificial Sequence Synthetic peptide
TAKTLAE

SEQ ID NO: 373 - protein, 9 aa, Artificial Sequence
  Description of Artificial Sequence Synthetic peptide
QHHYVTPYT

SEQ ID NO: 374 - protein, 114 aa, Artificial Sequence
  Description of Artificial Sequence Synthetic polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 375 - protein, 108 aa, Artificial Sequence
  Description of Artificial Sequence Synthetic polypeptide
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 376 - protein, 9 aa, Artificial Sequence
  Description of Artificial Sequence Synthetic peptide
SGFTFSSYD

SEQ ID NO: 377 - protein, 7 aa, Artificial Sequence
  Description of Artificial Sequence Synthetic peptide
SGGGTYT

SEQ ID NO: 378 - protein, 5 aa, Artificial Sequence
  Description of Artificial Sequence Synthetic peptide
ASMDY

SEQ ID NO: 379 - protein, 11 aa, Artificial Sequence
  Description of Artificial Sequence Synthetic peptide
RASQSIRRYLN

SEQ ID NO: 380 - protein, 7 aa, Artificial Sequence
  Description of Artificial Sequence Synthetic peptide
GASTLQS

SEQ ID NO: 381 - protein, 9 aa, Artificial Sequence
  Description of Artificial Sequence Synthetic peptide
QQSHSAPLT

SEQ ID NO: 382 - protein, 118 aa, Artificial Sequence
  Description of Artificial Sequence Synthetic polypeptide
QVQLQQPGAELVKPGASVKMSCKASGYTFTSYNMHWIKQTPGQGLEWIGDIYPGNGDTSYNQKFKGKATLTADKSSSTVYMQLSSLTSEDSAVYYCARVGGAFPMDYWGQGTSVTVSS

SEQ ID NO: 383 - protein, 111 aa, Artificial Sequence
  Description of Artificial Sequence Synthetic polypeptide
DIVLTQSPASLAVSLGQRATISCRASESVEYYGTSLMQWYQQKPGQPPKLLIYAASNVESGVPARFSGSGSGTDFSLNIHPVEEDDIAIYFCQQSRKDPSTFGGGTKLEIK

SEQ ID NO: 384 - protein, 5 aa, Artificial Sequence
  Description of Artificial Sequence Synthetic peptide
SYNMH

SEQ ID NO: 385 - protein, 17 aa, Artificial Sequence
  Description of Artificial Sequence Synthetic peptide
DIYPGNGDTSYNQKFKG

SEQ ID NO: 386 - protein, 9 aa, Artificial Sequence
  Description of Artificial Sequence Synthetic peptide
VGGAFPMDY

SEQ ID NO: 387 - protein, 15 aa, Artificial Sequence
  Description of Artificial Sequence Synthetic peptide
RASESVEYYGTSLMQ

SEQ ID NO: 388 - protein, 7 aa, Artificial Sequence
  Description of Artificial Sequence Synthetic peptide
AASNVES

SEQ ID NO: 389 - protein, 9 aa, Artificial Sequence
  Description of Artificial Sequence Synthetic peptide
QQSRKDPST

SEQ ID NO: 390 - protein, 114 aa, Artificial Sequence
  Description of Artificial Sequence Synthetic polypeptide
QVQLVQSGAEVKKPGASVKVSCKASGYTFTGYTMHWVRQAPGQGLEWIGYINPRSGYTEYNQKFKDRTTLTADKSTSTAYMELSSLRSEDTAVYYCARPWFAYWGQGTLVTVSS

SEQ ID NO: 391 - protein, 113 aa, Artificial Sequence
  Description of Artificial Sequence Synthetic polypeptide
DIVMTQSPAFLSVTPGEKVTITCKSSQSLLNSGNQKNYLTWYQQKPGQPPKLLIYWASTRESGVPDRFSGSGSGTDFTLTISSLQAEDVAVYYCQNDYSYPLTFGQGTKLEIK

SEQ ID NO: 392 - protein, 8 aa, Artificial Sequence
  Description of Artificial Sequence Synthetic peptide
GYTFTGYT

SEQ ID NO: 393 - protein, 7 aa, Artificial Sequence
  Description of Artificial Sequence Synthetic peptide
NPRSGYT

SEQ ID NO: 394 - protein, 7 aa, Artificial Sequence
  Description of Artificial Sequence Synthetic peptide
ARPWFAY

SEQ ID NO: 395 - protein, 17 aa, Artificial Sequence
  Description of Artificial Sequence Synthetic peptide
KSSQSLLNSGNQKNYLT

SEQ ID NO: 396 - protein, 7 aa, Artificial Sequence
  Description of Artificial Sequence Synthetic peptide
WASTRES

SEQ ID NO: 397 - protein, 9 aa, Artificial Sequence
  Description of Artificial Sequence Synthetic peptide
QNDYSYPLT

SEQ ID NO: 398 - protein, 136 aa, Artificial Sequence
  Description of Artificial Sequence Synthetic polypeptide
MNFGLSLIFLALILKGVQCEVQLVESGGDLVKSGGSLKLSCAASGFIFSSFGMSWVRQTPDKRLEWVATISSGGRNIYYLDSVKGRFTISRDNVKNILYLQMSGLKSEDSAMYYCAREGHYALDYCGQGTSVTVSS

SEQ ID NO: 399 - protein, 113 aa, Artificial Sequence
  Description of Artificial Sequence Synthetic polypeptide
DIVMTQSPSSLATSVGQRVTMSCKSSQNLLYSTDQKNYLAWFQQKPGQSPKLLLYFASIRESGVPDRFIGSGSGTDFTLTISSVQAEDLADYFCQQHYNTPPTFGGGTRLEIK

SEQ ID NO: 400 - protein, 5 aa, Artificial Sequence
  Description of Artificial Sequence Synthetic peptide
SFGMS

SEQ ID NO: 401 - protein, 17 aa, Artificial Sequence
  Description of Artificial Sequence Synthetic peptide
TISSGGRNIYYLDSVKG

SEQ ID NO: 402 - protein, 8 aa, Artificial Sequence
  Description of Artificial Sequence Synthetic peptide
EGHYALDY

SEQ ID NO: 403 - protein, 17 aa, Artificial Sequence
  Description of Artificial Sequence Synthetic peptide
KSSQNLLYSTDQKNYLA

SEQ ID NO: 404 - protein, 7 aa, Artificial Sequence
  Description of Artificial Sequence Synthetic peptide
FASIRES

SEQ ID NO: 405 - protein, 9 aa, Artificial Sequence
  Description of Artificial Sequence Synthetic peptide
QQHYNTPPT

SEQ ID NO: 406 - protein, 122 aa, Artificial Sequence
  Description of Artificial Sequence Synthetic polypeptide
EVQLVQSGSDLKKPGASVRVSCKASGYTFTSYPMNWVRQAPGHGLEWMGWINTNTGNPTYVQGFTGRFVFSLDTSVNTAYLQISSLKAEDTAVYFCARTGGHTYDSYAFDVWGQGTMVTVSS

SEQ ID NO: 407 - protein, 107 aa, Artificial Sequence
  Description of Artificial Sequence Synthetic polypeptide
DIQLTQSPTFLSASVGDRVTITCRASQVISSSLAWYQQNPGKAPKLLIYAASTLQSGVPSRFSGSGSGTEFTLTISSLQPEDFVTYYCQHLHGYPSNFGQGTKVEIK

SEQ ID NO: 408 - protein, 5 aa, Artificial Sequence
  Description of Artificial Sequence Synthetic peptide
SYPMN

SEQ ID NO: 409 - protein, 17 aa, Artificial Sequence
  Description of Artificial Sequence Synthetic peptide
WINTNTGNPTYVQGFTG

SEQ ID NO: 410 - protein, 13 aa, Artificial Sequence
  Description of Artificial Sequence Synthetic peptide
TGGHTYDSYAFDV

SEQ ID NO: 411 - protein, 11 aa, Artificial Sequence
  Description of Artificial Sequence Synthetic peptide
RASQVISSSLA

SEQ ID NO: 412 - protein, 7 aa, Artificial Sequence
  Description of Artificial Sequence Synthetic peptide
AASTLQS

SEQ ID NO: 413 - protein, 9 aa, Artificial Sequence
  Description of Artificial Sequence Synthetic peptide
QHLHGYPSN

SEQ ID NO: 414 - protein, 118 aa, Artificial Sequence
  Description of Artificial Sequence Synthetic polypeptide
DVQLQESGPGLVKPSQSLSLTCTVTGYSITSDYAWNWVRQFPGNKLEWMGYISYSGSTSYNPSLRSRISITRDTSKNQFFLQLNSVTTEDTATYYCARRQVGLGFAYWGQGTLVTVSS

SEQ ID NO: 415 - protein, 99 aa, Artificial Sequence
  Description of Artificial Sequence Synthetic polypeptide
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFG

SEQ ID NO: 416 - protein, 7 aa, Artificial Sequence
  Description of Artificial Sequence Synthetic peptide
TSDYAWN

SEQ ID NO: 417 - protein, 16 aa, Artificial Sequence
  Description of Artificial Sequence Synthetic peptide
YISYSGSTSYNPSLRS

SEQ ID NO: 418 - protein, 11 aa, Artificial Sequence
  Description of Artificial Sequence Synthetic peptide
ARRQVGLGFAY

SEQ ID NO: 419 - protein, 11 aa, Artificial Sequence
  Description of Artificial Sequence Synthetic peptide
KASQDVSTAVA

SEQ ID NO: 420 - protein, 7 aa, Artificial Sequence
  Description of Artificial Sequence Synthetic peptide
SASYRYT

SEQ ID NO: 421 - protein, 7 aa, Artificial Sequence
  Description of Artificial Sequence Synthetic peptide
QQHYSTP

SEQ ID NO: 422 - protein, 447 aa, Artificial Sequence
  Description of Artificial Sequence Synthetic polypeptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 423 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 424 - protein, 8 aa, Artificial Sequence
  Description of Artificial Sequence Synthetic peptide
GYTFTDSY

SEQ ID NO: 425 - protein, 5 aa, Artificial Sequence
  Description of Artificial Sequence Synthetic peptide
DNGDS

SEQ ID NO: 426 - protein, 10 aa, Artificial Sequence
  Description of Artificial Sequence Synthetic peptide
VLAPRWYFSV

SEQ ID NO: 427 - protein, 11 aa, Artificial Sequence
  Description of Artificial Sequence Synthetic peptide
RASQDISNYLN

SEQ ID NO: 428 - protein, 6 aa, Artificial Sequence
  Description of Artificial Sequence Synthetic peptide
TSRLRS

SEQ ID NO: 429 - protein, 9 aa, Artificial Sequence
  Description of Artificial Sequence Synthetic peptide
QQGHTLPPT

SEQ ID NO: 430 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 432 - protein, 8 aa, Artificial Sequence
  Description of Artificial Sequence Synthetic peptide
GGSFSSGY

SEQ ID NO: 433 - protein, 8 aa, Artificial Sequence
  Description of Artificial Sequence Synthetic peptide
SYNGITYH

SEQ ID NO: 434 - protein, 15 aa, Artificial Sequence
  Description of Artificial Sequence Synthetic peptide
ARYKYDYDGGHAMDY

SEQ ID NO: 435 - protein, 11 aa, Artificial Sequence
  Description of Artificial Sequence Synthetic peptide
RASQDISNYLN

SEQ ID NO: 436 - protein, 5 aa, Artificial Sequence
  Description of Artificial Sequence Synthetic peptide
TSKLH

SEQ ID NO: 437 - protein, 9 aa, Artificial Sequence
  Description of Artificial Sequence Synthetic peptide
QQGSALPWT

SEQ ID NO: 438 - protein, 446 aa, Artificial Sequence
  Description of Artificial Sequence Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 439 - protein, 218 aa, Artificial Sequence
  Description of Artificial Sequence Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 440 - protein, 8 aa, Artificial Sequence
  Description of Artificial Sequence Synthetic peptide
GYTFTDYY

SEQ ID NO: 441 - protein, 7 aa, Artificial Sequence
  Description of Artificial Sequence Synthetic peptide
YPGSGNT

SEQ ID NO: 442 - protein, 10 aa, Artificial Sequence
  Description of Artificial Sequence Synthetic peptide
ANYGNYWFAY

SEQ ID NO: 443 - protein, 15 aa, Artificial Sequence
  Description of Artificial Sequence Synthetic peptide
KASQSVDFDGDSYMN

SEQ ID NO: 444 - protein, 7 aa, Artificial Sequence
  Description of Artificial Sequence Synthetic peptide
AASNLES

SEQ ID NO: 445 - protein, 9 aa, Artificial Sequence
  Description of Artificial Sequence Synthetic peptide
QQSNEDPWT

SEQ ID NO: 446 - protein, 117 aa, Artificial Sequence
  Description of Artificial Sequence Synthetic polypeptide
QVQLVQSGAEVKKPGASVKVSCKASGYTFTDYYITWVRQAPGQGLEWMGWIYPGSGNTKYNEKFKGRVTMTVDTSISTAYMELSRLRSDDTAVYFCANYGNYWFAYWGQGTLVTVSS

SEQ ID NO: 447 - protein, 111 aa, Artificial Sequence
  Description of Artificial Sequence Synthetic polypeptide
DIVMTQSPDSLAVSLGERATINCKASQSVDFDGDSYMNWYQQKPGQPPKLLIYAASNLESGVPDRFSGSGSGTDFTLTISSLQAEDVAVYYCQQSNEDPWTFGQGTKVEIK

SEQ ID NO: 448 - protein, 8 aa, Artificial Sequence
  Description of Artificial Sequence Synthetic peptide
GYTFTDYY

SEQ ID NO: 449 - protein, 7 aa, Artificial Sequence
  Description of Artificial Sequence Synthetic peptide
YPGSGNT

SEQ ID NO: 450 - protein, 10 aa, Artificial Sequence
  Description of Artificial Sequence Synthetic peptide
ANYGNYWFAY

SEQ ID NO: 451 - protein, 15 aa, Artificial Sequence
  Description of Artificial Sequence Synthetic peptide
KASQSVDFDGDSYMN

SEQ ID NO: 452 - protein, 7 aa, Artificial Sequence
  Description of Artificial Sequence Synthetic peptide
AASNLES

SEQ ID NO: 453 - protein, 9 aa, Artificial Sequence
  Description of Artificial Sequence Synthetic peptide
QQSNEDPWT

SEQ ID NO: 454 - protein, 448 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 455 - protein, 216 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 456 - protein, 8 aa, Artificial Sequence
  Description of Artificial Sequence Synthetic peptide
GGSFSGYY

SEQ ID NO: 457 - protein, 6 aa, Artificial Sequence
  Description of Artificial Sequence Synthetic peptide
NHGGYV

SEQ ID NO: 458 - protein, 15 aa, Artificial Sequence
  Description of Artificial Sequence Synthetic peptide
ARDYGPGNYDWYFDL

SEQ ID NO: 459 - protein, 11 aa, Artificial Sequence
  Description of Artificial Sequence Synthetic peptide
RASQSVSSYLA

SEQ ID NO: 460 - protein, 9 aa, Artificial Sequence
  Description of Artificial Sequence Synthetic peptide
DASNRATGI

SEQ ID NO: 461 - protein, 11 aa, Artificial Sequence
  Description of Artificial Sequence Synthetic peptide
QQRSNWPPALT

SEQ ID NO: 462 - protein, 442 aa, Artificial Sequence
  Description of Artificial Sequence Synthetic polypeptide
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 463 - protein, 214 aa, Artificial Sequence
  Description of Artificial Sequence Synthetic polypeptide
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 464 - protein, 8 aa, Artificial Sequence
  Description of Artificial Sequence Synthetic peptide
GYSFSTYW

SEQ ID NO: 465 - protein, 7 aa, Artificial Sequence
  Description of Artificial Sequence Synthetic peptide
YPGDSYT

SEQ ID NO: 466 - protein, 9 aa, Artificial Sequence
  Description of Artificial Sequence Synthetic peptide
ARGYGIFDY

SEQ ID NO: 467 - protein, 11 aa, Artificial Sequence
  Description of Artificial Sequence Synthetic peptide
SGDNIGDQYAH

SEQ ID NO: 468 - protein, 7 aa, Artificial Sequence
  Description of Artificial Sequence Synthetic peptide
QDKNRPS

SEQ ID NO: 469 - protein, 11 aa, Artificial Sequence
  Description of Artificial Sequence Synthetic peptide
ATYTGFGSLAV

SEQ ID NO: 470 - protein, 447 aa, Artificial Sequence
  Description of Artificial Sequence Synthetic polypeptide
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 471 - protein, 216 aa, Artificial Sequence
  Description of Artificial Sequence Synthetic polypeptide
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 472 - protein, 8 aa, Artificial Sequence
  Description of Artificial Sequence Synthetic peptide
GFTFSSYA

SEQ ID NO: 473 - protein, 7 aa, Artificial Sequence
  Description of Artificial Sequence Synthetic peptide
SGSGGRT

SEQ ID NO: 474 - protein, 10 aa, Artificial Sequence
  Description of Artificial Sequence Synthetic peptide
ARLGYGRVDE

SEQ ID NO: 475 - protein, 13 aa, Artificial Sequence
  Description of Artificial Sequence Synthetic peptide
SGSLSNIGRNPVN

SEQ ID NO: 476 - protein, 7 aa, Artificial Sequence
  Description of Artificial Sequence Synthetic peptide
LDNLRLS

SEQ ID NO: 477 - protein, 11 aa, Artificial Sequence
  Description of Artificial Sequence Synthetic peptide
ATWDDSHPGWT

SEQ ID NO: 478 - protein, 121 aa, Artificial Sequence
  Description of Artificial Sequence Synthetic polypeptide
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 479 - protein, 107 aa, Artificial Sequence
  Description of Artificial Sequence Synthetic polypeptide
EIVLTQSPATLSLSPGERATLSCRASQGVSSYLAWYQQKPGQAPRLLIYDASNRATGIPARFSGSGPGTDFTLTISSLEPEDFAVYYCQQRSNWHLTFGGGTKVEIK

SEQ ID NO: 480 - protein, 5 aa, Artificial Sequence
  Description of Artificial Sequence Synthetic peptide
NYGMH

SEQ ID NO: 481 - protein, 16 aa, Artificial Sequence
  Description of Artificial Sequence Synthetic peptide
VILYDGSNKYYPDSVK

SEQ ID NO: 482 - protein, 12 aa, Artificial Sequence
  Description of Artificial Sequence Synthetic peptide
GGSSWYPDSFDI

SEQ ID NO: 483 - protein, 11 aa, Artificial Sequence
  Description of Artificial Sequence Synthetic peptide
RASQGVSSYLA

SEQ ID NO: 484 - protein, 7 aa, Artificial Sequence
  Description of Artificial Sequence Synthetic peptide
DASNRAT

SEQ ID NO: 485 - protein, 9 aa, Artificial Sequence
  Description of Artificial Sequence Synthetic peptide
QQRSNWHLT

SEQ ID NO: 486 - protein, 127 aa, Artificial Sequence
  Description of Artificial Sequence Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGIAAAGPPYYYYYYYMDVWGKGTTVTVSS

SEQ ID NO: 487 - protein, 107 aa, Artificial Sequence
  Description of Artificial Sequence Synthetic polypeptide
DIQMTQSPSSLSASVGDRVTITCRASQTIYNYLNWYQQKPGKAPKLLIYAASSLQSGVPSRFGGRGYGTDFTLTINSLQPEDFATYFCQQSYTSPLTFGQGTKVDIK

SEQ ID NO: 488 - protein, 7 aa, Artificial Sequence
  Description of Artificial Sequence Synthetic peptide
GFTFSSY

SEQ ID NO: 489 - protein, 6 aa, Artificial Sequence
  Description of Artificial Sequence Synthetic peptide
SYDGSN

SEQ ID NO: 490 - protein, 18 aa, Artificial Sequence
  Description of Artificial Sequence Synthetic peptide
GIAAAGPPYYYYYYYMDV

SEQ ID NO: 491 - protein, 8 aa, Artificial Sequence
  Description of Artificial Sequence Synthetic peptide
QTIYNYLN

SEQ ID NO: 492 - protein, 7 aa, Artificial Sequence
  Description of Artificial Sequence Synthetic peptide
AASSLQS

SEQ ID NO: 493 - protein, 9 aa, Artificial Sequence
  Description of Artificial Sequence Synthetic peptide
QQSYTSPLT

SEQ ID NO: 494 - protein, 118 aa, Artificial Sequence
  Description of Artificial Sequence Synthetic polypeptide
QVQLVESGGGVVQPGRSLRLSCAASGFTFSSYAMSWVRQAPGKGLEWVASISSGGTTYYPDSVKGRFTISRDNSKNTLYLQMNSLRAEDTAVYYCARVGGYYDSMDYWGQGTLVTVSS

SEQ ID NO: 495 - protein, 112 aa, Artificial Sequence
  Description of Artificial Sequence Synthetic polypeptide
  MOD_RES (31)..(31): Any amino acid
  MOD_RES (57)..(57): Any amino acid
EIVLTQSPGTLSLSPGERATLSCRASESVDXYGVSFMNWYQQKPGQAPRLLIYAASXQGSGIPDRFSGSGSGTDFTLTISRLEPEDFAVYYCQQTKEVTWTFGQGTKVEIKR

SEQ ID NO: 496 - protein, 8 aa, Artificial Sequence
  Description of Artificial Sequence Synthetic peptide
GFTFSSYA

SEQ ID NO: 497 - protein, 6 aa, Artificial Sequence
  Description of Artificial Sequence Synthetic peptide
SSGGTT

SEQ ID NO: 498 - protein, 12 aa, Artificial Sequence
  Description of Artificial Sequence Synthetic peptide
ARVGGYYDSMDY

SEQ ID NO: 499 - protein, 15 aa, Artificial Sequence
  Description of Artificial Sequence Synthetic peptide
  MOD_RES (8)..(8): Any amino acid
RASESVDXYGVSFMN

SEQ ID NO: 500 - protein, 7 aa, Artificial Sequence
  Description of Artificial Sequence Synthetic peptide
  MOD_RES (4)..(4): Any amino acid
AASXQGS

SEQ ID NO: 501 - protein, 9 aa, Artificial Sequence
  Description of Artificial Sequence Synthetic peptide
QQTKEVTWT

SEQ ID NO: 502 - protein, 473 aa, Artificial Sequence
  Description of Artificial Sequence Synthetic polypeptide
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYILLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 503 - protein, 240 aa, Artificial Sequence
  Description of Artificial Sequence Synthetic polypeptide
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 504 - protein, 223 aa, Artificial Sequence
  Description of Artificial Sequence Synthetic polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 505 - protein, 387 aa, Artificial Sequence
  Description of Artificial Sequence Synthetic polypeptide
MREPLEAFKLADLDFRKSSLASGWRMASGAFTMDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 506 - protein, 522 aa, Artificial Sequence
  Description of Artificial Sequence Synthetic polypeptide
MGREELFLTFSFSSGFQESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 507 - protein, 301 aa, Artificial Sequence
  Description of Artificial Sequence Synthetic polypeptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 508 - protein, 398 aa, Artificial Sequence
  Description of Artificial Sequence Synthetic polypeptide
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 509 - protein, 273 aa, Artificial Sequence
  Description of Artificial Sequence Synthetic polypeptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 510 - protein, 244 aa, Artificial Sequence
  Description of Artificial Sequence Synthetic polypeptide
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 511 - protein, 277 aa, Artificial Sequence
  Description of Artificial Sequence Synthetic polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 512 - protein, 595 aa, Artificial Sequence
  Description of Artificial Sequence Synthetic polypeptide
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 513 - protein, 255 aa, Artificial Sequence
  Description of Artificial Sequence Synthetic polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 514 - protein, 296 aa, Artificial Sequence
  Description of Artificial Sequence Synthetic polypeptide
MTLNNVTMRQGTVGMQPQQQRWSIPADGRHLMVQKEPHQYSHRNRHSATPEDHCRRSWSSDSTDSVISSESGNTYYRVVLIGEQGVGKSTLANIFAGVHDSMDSDCEVLGEDTYERTLMVDGESATIILLDMWENKGENEWLHDHCMQVGDAYLIVYSITDRASFEKASELRIQLRRARQTEDIPIILVGNKSDLVRCREVSVSEGRACAVVFDCKFIETSAAVQHNVKELFEGIVRQVRLRRDSKEKNERRLAYQKRKESMPRKARRFWGKIVAKNNKNMAFKLKSKSCHDLSVL

SEQ ID NO: 515 - protein, 574 aa, Artificial Sequence
  Description of Artificial Sequence Synthetic polypeptide
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 516 - protein, 255 aa, Artificial Sequence
  Description of Artificial Sequence Synthetic polypeptide
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 517 - protein, 9 aa, Artificial Sequence
  Description of Artificial Sequence Synthetic peptide
GTFSSYAIS

SEQ ID NO: 518 - protein, 17 aa, Artificial Sequence
  Description of Artificial Sequence Synthetic peptide
GIIPIFGTANYAQKFQG

SEQ ID NO: 519 - protein, 19 aa, Artificial Sequence
  Description of Artificial Sequence Synthetic peptide
ARRGRKASGSFYYYYGMDV

SEQ ID NO: 520 - protein, 17 aa, Artificial Sequence
  Description of Artificial Sequence Synthetic peptide
ESSQSLLNSGNQKNYLT

SEQ ID NO: 521 - protein, 7 aa, Artificial Sequence
  Description of Artificial Sequence Synthetic peptide
WASTRES

SEQ ID NO: 522 - protein, 120 aa, Artificial Sequence
  Description of Artificial Sequence Synthetic polypeptide
EVQLVESGGGLVQPGRSLRLSCTASGFTFTDNYMSWVRQAPGKGLEWVGFIRNKANGYTTEYAASVKGRFTISRDNSKSIAYLQMNSLKTEDTAVYYCARDVGSNYFDYWGQGTLVTVSS

SEQ ID NO: 523 - protein, 9 aa, Artificial Sequence
  Description of Artificial Sequence Synthetic peptide
FTFTDNYMS

SEQ ID NO: 524 - protein, 17 aa, Artificial Sequence
  Description of Artificial Sequence Synthetic peptide
FIRNKANGYTTEYAASV

SEQ ID NO: 525 - protein, 11 aa, Artificial Sequence
  Description of Artificial Sequence Synthetic peptide
ARDVGSNYFDY

SEQ ID NO: 526 - protein, 112 aa, Artificial Sequence
  Description of Artificial Sequence Synthetic polypeptide
DIVMTQSPSSLAVSLGERATMSCKSSQSLFNSRTRKNYLAWYQQKPGQSPKLLIYWASARDSGVPARFTGSGSETYFTLTISRVQAEDLAVYYCMQSFNLRTFGQGTKVEIK

SEQ ID NO: 527 - protein, 17 aa, Artificial Sequence
  Description of Artificial Sequence Synthetic peptide
KSSQSLFNSRTRKNYLA

SEQ ID NO: 528 - protein, 7 aa, Artificial Sequence
  Description of Artificial Sequence Synthetic peptide
WASARDS

SEQ ID NO: 529 - protein, 8 aa, Artificial Sequence
  Description of Artificial Sequence Synthetic peptide
MQSFNLRT

(19) United States
(12) Patent Application Publication
(10) Pub. No.: US 2020/0231679 A1
(43) Pub. Date: Jul. 23, 2020

(54) PROTEINS BINDING NKG2D, CD16 AND A TUMOR-ASSOCIATED ANTIGEN

(71) Applicant: Dragonfly Therapeutics, Inc., Waltham, MA (US)

(72) Inventors: Gregory P. Chang, Medford, MA (US); Ann F. Cheung, Lincoln, MA (US); William Haney, Wayland, MA (US); Bradley M. Lunde, Lebanon, NH (US); Bianka Prinz, Lebanon, NH (US); Nicolai Wagtmann, Concord, MA (US); Jinyan Du, Waltham, MA (US)

(21) Appl. No.: 16/639,150

(22) PCT Filed: Aug. 23, 2018
(86) PCT No.: PCT/US2018/047714

§ 371 Date: Feb. 14, 2020

Related U.S. Application Data
(60) Provisional application No. 62/549,201, filed on Aug. 23, 2017.
(60) Provisional application No. 62/558,514, filed on Sep. 14, 2017.
(60) Provisional application No. 62/558,509, filed on Sep. 14, 2017.
(60) Provisional application No. 62/558,510, filed on Sep. 14, 2017.
(60) Provisional application No. 62/558,511, filed on Sep. 14, 2017.
(60) Provisional application No. 62/566,828, filed on Oct. 2, 2017.
(60) Provisional application No. 62/581,357, filed on Nov. 3, 2017.
(60) Provisional application No. 62/608,384, filed on Dec. 20, 2017.

(51) Int. Cl. C07K 16/28

(52) U.S. Cl. CPC C07K 16/2851; C07K 16/283; C07K 16/2866; C07K 2317/21; C07K 2317/31; C07K 2317/33; C07K 2319/32; C07K 2317/55; C07K 2317/64; C07K 2317/732; C07K 2317/76; C07K 2317/94; C07K 2319/30; C07K 2317/526

(57) ABSTRACT

Multi-specific binding proteins that bind the NKG2D receptor, CD 16, and a tumor-associated antigen are described, as well as pharmaceutical compositions and therapeutic methods useful for the treatment of cancer.

### CROSS-REFERENCE TO RELATED APPLICATIONS

This application claims the benefit of and priority to U.S. Provisional Patent Application No. 62/549,201, filed Aug. 23, 2017, the disclosure of which is hereby incorporated by reference in its entirety for all purposes; U.S. Provisional Patent Application No. 62/558,509, filed Sep. 14, 2017, the disclosure of which is hereby incorporated by reference in its entirety for all purposes; U.S. Provisional Patent Application No. 62/558,510, filed Sep. 14, 2017; U.S. Provisional Patent Application No. 62/558,511, filed Sep. 14, 2017, the disclosure of which is hereby incorporated by reference in its entirety for all purposes; U.S. Provisional Patent Application No. 62/558,514, filed Sep. 14, 2017, the disclosure of which is hereby incorporated by reference in its entirety for all purposes; U.S. Provisional Patent Application No. 62/566,828, filed Oct. 2, 2017, the disclosure of which is hereby incorporated by reference in its entirety for all purposes; U.S. Provisional Patent Application No. 62/581,357, filed Nov. 3, 2017, the disclosure of which is hereby incorporated by reference in its entirety for all purposes; and U.S. Provisional Patent Application No. 62/608,384, filed Dec. 20, 2017, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Aug. 22, 2018, is named DFY-034WO_SL.txt and is 448,772 bytes in size.

### FIELD OF THE INVENTION

The invention relates to multi-specific binding proteins that bind to NKG2D, CD16, and a tumor-associated antigen.

### BACKGROUND

Cancer continues to be a significant health problem despite the substantial research efforts and scientific advances reported in the literature for treating this disease. Some of the most frequently diagnosed cancers include prostate cancer, breast cancer, lung cancer, and colorectal cancer. Prostate cancer is the most common form of cancer in men. Breast cancer remains a leading cause of death in women. Blood and bone marrow cancers are also frequently diagnosed cancer types, including multiple myelomas, leukemia, and lymphomas. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, for example WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-gamma and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

Chemokines mediate numerous physiological and pathological processes related primarily to cell homing and migration. The human chemokine system currently includes more than 40 chemokines and 18 chemokine receptors. CXCR4 is one of the most studied chemokine receptors. It is a 352 amino acid rhodopsin-like G-protein coupled receptor that selectively binds chemokine CXCL12, and mediates chemotaxis, enhanced intracellular calcium, cell adhesion, survival, proliferation, and gene transcription through multiple divergent pathways. CXCR4 is overexpressed in more than 23 different types of human cancers including kidney, lung, brain, prostate, breast, pancreas, ovarian, and melanomas and this aberrant expression strongly promotes tumor proliferation, migration and invasion through multiple signal pathways. CXCR4 is also important in the homing of malignant cells, such as in acute myeloid leukemia and multiple myeloma, to niches in the bone marrow, which have been described to promote resistance to chemotherapy.

Regulatory T cells (T_{regs}) protect against autoimmunity, but in cancer, T_{regs} infiltrate even the earliest neoplastic lesions and undermine anti-tumor effector T cells. T_{reg} development and homeostasis are critically dependent on interleukin-2 (IL-2), and most T_{regs} express high levels of CD25, the cell surface a chain of the IL-2 receptor. CD25 monoclonal antibody have been shown to deplete CD25⁺T_{regs} in vivo and enhance tumor immunity and immunotherapy. Therefore, CD25 blockage represents an approach to circumvent a major element of immune suppression in patients with cancer, including acute myeloid leukemia, chronic lymphocytic leukemia, glioblastoma, bladder cancer, colon cancer, germ cell tumors, lung cancer, osteosarcoma, melanoma, ovarian cancer, multiple myeloma, head and neck cancer, renal cell cancer, and breast cancer.

Antigens highly expressed on T_{regs} can be exploited in an anti-cancer therapy that targets a specific antigen for depletion of tumor resident T_{regs} and thereby relieves immune suppression in patients with cancer. These antigens include CCR8, which specifically binds and responds to cytokines of the CC chemokine family; CD7, also known as leu-9 or GP40, which is a cell surface glycoprotein; CTLA4, also known as CD152, which is a protein receptor and functions as an immune checkpoint; CX3CR1, also known as the fractalkine receptor or G-protein coupled receptor 13 (GPR13), which is a receptor for chemokine CX3CL1; ENTPD1, also known as CD39 or NTPDasel, which is an ectonucleotidase that catalyzes the hydrolysis of γ- and β-phosphate residues of triphospho- and diphosphonucleosides to the monophosphonucleoside derivative; HAVCR2, also known as TIM-3; IL1R2, also known as CD121b, which is a receptor for interleukin-1α (ILIA), interleukin-113 (IL1B), and interleukin 1 receptor antagonist (IL1Ra), preventing them from binding to their regular receptors and thereby inhibiting the transduction of their signaling; PDCD1LG2, also known as B7DC, CD273 or PD-L2, which is a ligand of PD-1 and negatively regulates T cell activation; TIGIT, which is an immune receptor on T_{regs} and functions as an immune checkpoint; TNFRSF4, also known as CD134 or OX40; TNFRSF8, also known as CD30; TNFRSF9, also known as CD137; GEM, a member of the RAD/GEM family of GTP-binding proteins; NT5E, also known as CD73, which converts AMP to adenosine; and TNFRSF18, also known as GITR or CD357.

VLA4, CD44, CD13, CD15, CD47, and CD81 are associated with a variety of tumors. Very late antigen-4 (VLA-4) is a key adhesion molecule that acts as a receptor for the extracellular matrix protein fibronectin, and the cellular counter-receptor VCAM-1. It is expressed by numerous cells of hematopoietic origin and possesses a key function in the cellular immune response, e.g., by mediating leukocyte tethering, rolling, binding, and finally transmigration of the vascular wall at inflammatory sites. In addition, VLA-4 is expressed in leukemic cells and different solid tumors such as acute myeloid leukemia, multiple myeloma, chronic lymphocytic leukemia, breast cancer, glioblastoma.

CD44 is a transmembrane glycoprotein that has various functions in cell-cell interactions, cell adhesion and migration. It is also abundantly expressed in several cancers, including acute myeloid leukemia, breast cancer, head and neck cancer, ovarian cancer, prostate cancer, and melanoma.

CD13, also known as aminopeptidase N, is a Zn²⁺dependent membrane-bound ectopeptidase that degrades preferentially proteins and peptides with a N-terminal neutral amino acid. CD13 has been associated with malignant development, such as tumor cell invasion, differentiation, proliferation and apoptosis, motility and angiogenesis in acute myeloid leukemia, lung cancer, pancreatic cancer, liver cancer, and gastric cancer.

CD15 (3-fucosyl-N-acetyl-lactosamine) is a carbohydrate adhesion molecule that can be expressed on glycoproteins, glycolipids and proteoglycans. It is expressed in patients with acute myeloid leukemia, Hodgkin lymphoma, chronic lymphocytic leukemia, acute lymphoblastic leukemia, lung cancer and thyroid cancer.

CD47 (also known as integrin-associated protein) is a ubiquitously expressed glycoprotein of the immunoglobulin superfamily that plays a critical role in self-recognition.

Various solid and hematologic cancers exploit CD47 expression in order to evade immunological eradication, and its overexpression is clinically correlated with poor prognoses. It has been demonstrated that overexpression of CD47 occurs in nearly all types of tumors, some of which include acute myeloid leukemia, multiple myeloma, B cell lymphoma, T cell lymphoma, ovarian cancer, lung cancer, bladder cancer, and breast cancer.

CD81, is a cell surface glycoprotein that is known to complex with integrins. It is a member of the tetraspanin family, most of which are cell-surface proteins that are characterized by the presence of four hydrophobic domains, and mediate signal transduction events that play a role in the regulation of cell development, activation, growth and motility. CD81 participates in a variety of important cellular processes such as membrane organization, protein trafficking, cellular fusion and cell-cell interactions. CD81 has also been shown to contribute to tumor growth and metastasis, and to be expressed in most types of cancer, including acute myeloid leukemia, multiple myeloma, lymphoma, breast, lung, prostate, melanoma, and brain cancer.

CD23 is a type II integral membrane protein belonging to the calcium-dependent lectin superfamily. It is found on mature B cells, activated macrophages, eosinophils, follicular dendritic cells, and platelets. CD23 is also overexpressed in most B cell malignancies including chronic lymphocytic leukemia and Non-Hodgkin lymphoma.

CD40 is a molecule of the family of tumor necrosis factor receptors (TNFR), which is expressed throughout B-cell development and is implicated in cell survival and differentiation. The broad range of expression of CD40 on normal healthy cells translates to its extensive expression on a variety of tumors. It has been shown that CD40 is widely expressed on melanoma, prostate, lung cancers, and carcinomas of the nasopharynx, bladder, cervix, ovary and kidney. CD40 expression has also been reported on most B cell malignancies and other hematologic malignancies, such as non-Hodgkin lymphomas, Hodgkin lymphomas, chronic lymphocytic leukemia, multiple myeloma, diffuse large B cell lymphoma, and follicular lymphoma.

CD70 is a member of the tumor necrosis factor superfamily expressed primarily on activated lymphocytes. CD70 interacts with CD27 to regulate B and T cell functions. Among normal, non-lymphoid tissues, CD70 is only expressed on stromal cells of the thymic medulla and mature dendritic cells. CD70 is also expressed constitutively on a subset of B cell malignancies including Non-Hodgkin lymphoma and chronic lymphocytic leukemia, T cell lymphoma, renal cancer, glioblastoma, and head and neck cancer.

The CD79a protein together with the related CD79b protein, forms a dimer associated with membrane-bound immunoglobulin in B-cells, forming the B-cell antigen receptor (BCR). The CD79a/b heterodimer plays multiple and diverse roles in B cell development and function. It associates non-covalently with the immunoglobulin heavy chain through its transmembrane region, thus forming the BCR along with the immunoglobulin light chain. Association of the CD79a/b heterodimer with the immunoglobulin heavy chain is required for surface expression of the BCR and BCR induced calcium flux and protein tyrosine phosphorylation. The CD79a/b protein is present on the surface of B-cells throughout their life cycle, and is absent on all other healthy cells. The protein remains present when B-cells transform into active plasma cells, and is also present in virtually all B-cell malignancies, including B-cell lymphomas, Non-Hodgkin lymphoma, chronic lymphocytic leukemia, multiple myeloma, diffuse large B cell lymphoma, and follicular lymphoma.

CD80 is a member of the B7 family of immune coregulatory proteins that mediate both immune activation and suppression. CD80 in particular has recently been shown to play an important role in supporting immune suppression through interactions with B7-H1. It has been shown that CD80 is expressed on malignant B cells in essentially all cases of follicular lymphoma, the majority of cases of diffuse large B-cell lymphoma, marginal zone lymphoma, mantle cell lymphoma, Non-Hodgkin lymphoma, and chronic lymphocytic leukemia.

CRLF2 is a type I cytokine receptor also known as thymic stromal lymphopoietin (TSLP) receptor (TSLPR). It forms a functional complex with TSLP and IL7R, capable of stimulating cell proliferation through activation of STAT3, STATS and JAK2 pathways and is implicated in the development of the hematopoietic system. It has been shown that CRLF2 is overexpressed in B cell malignancies including acute lymphoblastic leukemia, Non-Hodgkin lymphoma, chronic lymphocytic leukemia.

Multiple myeloma is a cancer of plasma cells, a type of white blood cells responsible for producing antibodies. Surface antigens SLAMF7, CD138 and CD38 are universally overexpressed in multiple myeloma. SLAMF7 (also named CD319) is a member of the signaling lymphocytic activation molecule (SLAM) family receptors, and plays an important role in immune cell regulation. CD138 is a heparin sulphate proteoglycan, specific for terminally differentiated normal plasma cells. It is highly expressed in multiple myeloma, controlling tumor cell survival, growth, adhesion and bone cell differentiation. CD38 is a multifunctional ectoenzyme that catalyzes the synthesis and hydrolysis of cyclic ADP-ribose (cADPR) from NAD⁺ to ADP-ribose. Monoclonal antibodies targeting SLAMF7, CD138 or CD38 have been used as therapies for multiple myeloma.

T-cell lymphomas and leukemias are aggressive, treatment-resistant cancers with poor prognosis. The T-cell receptor, or TCR, is a molecule found on the surface of T cells, or T lymphocytes that is responsible for recognizing fragments of antigen as peptides bound to major histocompatibility complex (MHC) molecules. The TCR is composed of two different protein chains. In humans, in 95% of T cells the TCR consists of an alpha (α) chain and a beta (β) chain, whereas in 5% of T cells the TCR consists of gamma and delta (γ/δ) chains. The β-constant region of TCR comprises 2 functionally identical genes: TRBC1 (T cell receptor beta constant 1) and TRBC2 (T cell receptor beta constant 2). Each T-cell expresses only one of these. Hence, normal T-cells will be a mixture of individual cells expressing either TRBC1 or 2. A clonal T-cell cancer expresses TRBC1 or TRBC2 in its entirety, which can be exploited to treat T cell cancer.

Leukocyte immunoglobulin-like receptors (LILR) are a family of at least 13 receptors mainly expressed on lymphoid and myelomonocytic cells. They are divided into two subfamilies LILRBs and LILRAs, which are involved in the inhibition and stimulation of the immune system respectively. LILRBs have 5 members LILRB1-LILRB5, and they are predominantly expressed in hematopoietic lineage cells and to suppress activation of various types of immune cells. In addition to leukocytes, LILRBs and related receptors are expressed by tumor cells and were suggested to have direct tumor-sustaining activity. For example, LILRB1 is expressed on human acute myeloid leukemia (AML) cells (especially in monocytic AML cells), neoplastic B cells (including B cell leukemia, B cell lymphoma, and multiple myeloma cells), T cell leukemia and lymphoma cells, and gastric cancer cells. LILRB2, also known as LIR-2, ILT-4, MIR-10, and CD85d, is expressed on AML cells, e.g., the monocytic subtype, chronic lymphoblastic leukemia (CLL) cells, primary ductal and lobular breast cancer cells, and human non-small cell lung cancer cells. LILRB3 is expressed on myeloid leukemia, B lymphoid leukemia, and myeloma cells. LILRB4 is expressed on AML cells, e.g., the M4 and the M5 subtype, and about 50% of B cell chronic lymphocytic leukemia (B-CLL) cells. LILRBs are also specifically expressed or up-regulated on lung cancer, gastric cancer, breast cancer, and pancreas cancer cells.

### SUMMARY

The invention provides multi-specific binding proteins that bind to a tumor-associated antigen (selected from any one of the antigens provided in Table 15) and to the NKG2D receptor and CD16 receptor on natural killer cells. Such proteins can engage more than one kind of NK activating receptor, and may block the binding of natural ligands to NKG2D. In certain embodiments, the proteins can agonize NK cells in humans, and in other species such as rodents and cynomolgus monkeys. Various aspects and embodiments of the invention are described in further detail below.

Accordingly, one aspect of the invention provides a protein that incorporates a first antigen-binding site that binds NKG2D; a second antigen-binding site that binds CXCR4; and an antibody Fc domain, a portion thereof sufficient to bind CD16, or a third antigen-binding site that binds CD16. The antigen-binding sites may each incorporate an antibody heavy chain variable domain and an antibody light chain variable domain (e.g. arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

The invention provides multi-specific binding proteins that bind the NKG2D receptor, CD16, and an antigen selected from CXCR4, CD25, VLA4, CD44, CD13, CD15, CD47, CD81, CD23, CD40, CD70, CD79a, CD79b, CD80, CRLF2, SLAMF7, CD38, CD138, T-cell receptor beta-1 chain C region (TRBC1), T-cell receptor beta-2 chain C region (TRBC2), a leukocyte immunoglobulin-like receptor family member selected from LILRB1, LILRB2, LILRB3, LILRB4, LILRB5, LILRA1, LILRA2, LILRA3, LILRA4, LILRA5, and LILRA6, a regulatory T cell expressing protein selected from CC chemokine receptor 8 (CCR8), Cluster of Differentiation 7 (CD7), cytotoxic T-lymphocyte-associated protein 4 (CTLA4), CX3C chemokine receptor 1 (CX3CR1), Ectonucleoside Triphosphate Diphosphohydrolase-1 (ENTPD1), hepatitis A virus cellular receptor 2 (HAVCR2), interleukin 1 receptor type II (IL-1R2), programmed cell death 1 ligand 2 (PDCD1LG2), T cell immunoreceptor with Ig and ITIM domains (TIGIT), tumor necrosis factor receptor superfamily member 4 (TNFRSF4), tumor necrosis factor receptor superfamily member 8 (TNFRSF8), tumor necrosis factor receptor superfamily member 9 (TNFRSF9), GTP-binding protein GEM, ecto-5′-nucleotidase (NT5E), and tumor necrosis factor superfamily member 18 (TNFRSF18).

The first antigen-binding site, which binds to NKG2D, in some embodiments, can incorporate a heavy chain variable domain related to SEQ ID NO:1, such as by having an amino acid sequence at least 90% (e.g., 90%, 91%, 92%, 93%, 94%, 95%, 96%, 97%, 98%, 99%, or 100%) identical to SEQ ID NO:1, and/or incorporating amino acid sequences identical to the CDR1 (SEQ ID NO:105), CDR2 (SEQ ID NO:106), and CDR3 (SEQ ID NO:107) sequences of SEQ ID NO:1. The heavy chain variable domain related to SEQ ID NO:1 can be coupled with a variety of light chain variable domains to form an NKG2D binding site. For example, the first antigen-binding site that incorporates a heavy chain variable domain related to SEQ ID NO:1 can further incorporate a light chain variable domain selected from any one of the sequences related to SEQ ID NOs:2, 4, 6, 8, 10, 12, 14, 16, 18, 20, 22, 24, 26, 28, 30, 32, 34, 36, 38, and 40. For example, the first antigen-binding site incorporates a heavy chain variable domain with amino acid sequences at least 90% (e.g., 90%, 91%, 92%, 93%, 94%, 95%, 96%, 97%, 98%, 99%, or 100%) identical to SEQ ID NO:1 and a light chain variable domain with amino acid sequences at least 90% (e.g., 90%, 91%, 92%, 93%, 94%, 95%, 96%, 97%, 98%, 99%, or 100%) identical to any one of the sequences selected from SEQ ID NOs:2, 4, 6, 8, 10, 12, 14, 16, 18, 20, 22, 24, 26, 28, 30, 32, 34, 36, 38, and 40.

Alternatively, the first antigen-binding site can incorporate a heavy chain variable domain related to SEQ ID NO:41 and a light chain variable domain related to SEQ ID NO:42. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:41, and/or incorporate amino acid sequences identical to the CDR1 (SEQ ID NO:43), CDR2 (SEQ ID NO:44), and CDR3 (SEQ ID NO:45) sequences of SEQ ID NO:41. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:42, and/or incorporate amino acid sequences identical to the CDR1 (SEQ ID NO:46), CDR2 (SEQ ID NO:47), and CDR3 (SEQ ID NO:48) sequences of SEQ ID NO:42.

In other embodiments, the first antigen-binding site can incorporate a heavy chain variable domain related to SEQ ID NO:49 and a light chain variable domain related to SEQ ID NO:50. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:49, and/or incorporate amino acid sequences identical to the CDR1 (SEQ ID NO:51), CDR2 (SEQ ID NO:52), and CDR3 (SEQ ID NO:53) sequences of SEQ ID NO:49. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:50, and/or incorporate amino acid sequences identical to the CDR1 (SEQ ID NO:54), CDR2 (SEQ ID NO:55), and CDR3 (SEQ ID NO:56) sequences of SEQ ID NO:50.

Alternatively, the first antigen-binding site can incorporate a heavy chain variable domain related to SEQ ID NO:57 and a light chain variable domain related to SEQ ID NO:58, such as by having amino acid sequences at least 90% (e.g., 90%, 91%, 92%, 93%, 94%, 95%, 96%, 97%, 98%, 99%, or 100%) identical to SEQ ID NO:57 and at least 90% (e.g., 90%, 91%, 92%, 93%, 94%, 95%, 96%, 97%, 98%, 99%, or 100%) identical to SEQ ID NO:58, respectively.

In another embodiment, the first antigen-binding site can incorporate a heavy chain variable domain related to SEQ ID NO:59 and a light chain variable domain related to SEQ ID NO:60, For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:59, and/or incorporate amino acid sequences identical to the CDR1 (SEQ ID NO:517), CDR2 (SEQ ID NO:518), and CDR3 (SEQ ID NO:519) sequences of SEQ ID NO:59. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:60, and/or incorporate amino acid sequences identical to the CDR1 (SEQ ID NO:520), CDR2 (SEQ ID NO:521), and CDR3 (SEQ ID NO:355) sequences of SEQ ID NO:60.

The first antigen-binding site, which binds to NKG2D, in some embodiments, can incorporate a heavy chain variable domain related to SEQ ID NO:61 and a light chain variable domain related to SEQ ID NO:62. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:61, and/or incorporate amino acid sequences identical to the CDR1 (SEQ ID NO:63), CDR2 (SEQ ID NO:64), and CDR3 (SEQ ID NO:65) sequences of SEQ ID NO:61. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:62, and/or incorporate amino acid sequences identical to the CDR1 (SEQ ID NO:66), CDR2 (SEQ ID NO:67), and CDR3 (SEQ ID NO:68) sequences of SEQ ID NO:62.

In some embodiments, the first antigen-binding site can incorporate a heavy chain variable domain related to SEQ ID NO:69 and a light chain variable domain related to SEQ ID NO:70. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:69, and/or incorporate amino acid sequences identical to the CDR1 (SEQ ID NO:71), CDR2 (SEQ ID NO:72), and CDR3 (SEQ ID NO:73) sequences of SEQ ID NO:69. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:74), CDR2 (SEQ ID NO:75), and CDR3 (SEQ ID NO:76) sequences of SEQ ID NO:70.

In some embodiments, the first antigen-binding site can incorporate a heavy chain variable domain related to SEQ ID NO:77 and a light chain variable domain related to SEQ ID NO:78. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:77, and/or incorporate amino acid sequences identical to the CDR1 (SEQ ID NO:79), CDR2 (SEQ ID NO:80), and CDR3 (SEQ ID NO:81) sequences of SEQ ID NO:77. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:82), CDR2 (SEQ ID NO:83), and CDR3 (SEQ ID NO:84) sequences of SEQ ID NO:78.

In some embodiments, the first antigen-binding site can incorporate a heavy chain variable domain related to SEQ ID NO:85 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:85, and/or incorporate amino acid sequences identical to the CDR1 (SEQ ID NO:87), CDR2 (SEQ ID NO:88), and CDR3 (SEQ ID NO:89) sequences of SEQ ID NO:85. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:93 and a light chain variable domain related to SEQ ID NO:94. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:93, and/or incorporate amino acid sequences identical to the CDR1 (SEQ ID NO:95), CDR2 (SEQ ID NO:96), and CDR3 (SEQ ID NO:97) sequences of SEQ ID NO:93. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:98), CDR2 (SEQ ID NO:99), and CDR3 (SEQ ID NO:100) sequences of SEQ ID NO:94.

In some embodiments, the first antigen-binding site can incorporate a heavy chain variable domain related to SEQ ID NO:101 and a light chain variable domain related to SEQ ID NO:102, such as by having amino acid sequences at least 90% (e.g., 90%, 91%, 92%, 93%, 94%, 95%, 96%, 97%, 98%, 99%, or 100%) identical to SEQ ID NO:101 and at least 90% (e.g., 90%, 91%, 92%, 93%, 94%, 95%, 96%, 97%, 98%, 99%, or 100%) identical to SEQ ID NO:102, respectively.

In some embodiments, the first antigen-binding site can incorporate a heavy chain variable domain related to SEQ ID NO:103 and a light chain variable domain related to SEQ ID NO:104, such as by having amino acid sequences at least 90% (e.g., 90%, 91%, 92%, 93%, 94%, 95%, 96%, 97%, 98%, 99%, or 100%) identical to SEQ ID NO:103 and at least 90% (e.g., 90%, 91%, 92%, 93%, 94%, 95%, 96%, 97%, 98%, 99%, or 100%) identical to SEQ ID NO:104, respectively.

In some embodiments, the second antigen-binding site can bind to CXCR4 and can incorporate a heavy chain variable domain related to SEQ ID NO:109 and a light chain variable domain related to SEQ ID NO:110. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:109, and/or incorporate amino acid sequences identical to the CDR1 (SEQ ID NO:111), CDR2 (SEQ ID NO:112), and CDR3 (SEQ ID NO:113) sequences of SEQ ID NO:109 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:110, and/or incorporate amino acid sequences identical to the CDR1 (SEQ ID NO:114), CDR2 (SEQ ID NO:115), and CDR3 (SEQ ID NO:116) sequences of SEQ ID NO:110.

In some embodiments, the second antigen-binding site can bind to CXCR4 and can incorporate a heavy chain variable domain related to SEQ ID NO:117 and a light chain variable domain related to SEQ ID NO:118. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:117, and/or incorporate amino acid sequences identical to the CDR1 (SEQ ID NO:119), CDR2 (SEQ ID NO:120), and CDR3 (SEQ ID NO:121) sequences of SEQ ID NO:117 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:118, and/or incorporate amino acid sequences identical to the CDR1 (SEQ ID NO:122), CDR2 (SEQ ID NO:123), and CDR3 (SEQ ID NO:124) sequences of SEQ ID NO:118.

In some embodiments, the second antigen-binding site can bind to CXCR4 and can incorporate a heavy chain variable domain related to SEQ ID NO:125 and a light chain variable domain related to SEQ ID NO:126. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:125, and/or incorporate amino acid sequences identical to the CDR1 (SEQ ID NO:127), CDR2 (SEQ ID NO:128), and CDR3 (SEQ ID NO:129) sequences of SEQ ID NO:125 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:126, and/or incorporate amino acid sequences identical to the CDR1 (SEQ ID NO:130), CDR2 (SEQ ID NO:131), and CDR3 (SEQ ID NO:132) sequences of SEQ ID NO:126.

In some embodiments, the second antigen-binding site can bind to CXCR4 and can incorporate a heavy chain variable domain related to SEQ ID NO:522 and a light chain variable domain related to SEQ ID NO:526. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:522, and/or incorporate amino acid sequences identical to the CDR1 (SEQ ID NO:523), CDR2 (SEQ ID NO:524), and CDR3 (SEQ ID NO:525) sequences of SEQ ID NO:522 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:526, and/or incorporate amino acid sequences identical to the CDR1 (SEQ ID NO:527), CDR2 (SEQ ID NO:528), and CDR3 (SEQ ID NO:529) sequences of SEQ ID NO:526.

In some embodiments, the second antigen-binding site can bind to CD25 and can incorporate a heavy chain variable domain related to SEQ ID NO:134 and a light chain variable domain related to SEQ ID NO:135. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:134, and/or incorporate amino acid sequences identical to the CDR1 (SEQ ID NO:136), CDR2 (SEQ ID NO:137), and CDR3 (SEQ ID NO:138) sequences of SEQ ID NO:134 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:135, and/or incorporate amino acid sequences identical to the CDR1 (SEQ ID NO:139), CDR2 (SEQ ID NO:140), and CDR3 (SEQ ID NO:141) sequences of SEQ ID NO:135.

In some embodiments, the second antigen-binding site can bind to CD25 and can incorporate a heavy chain variable domain related to SEQ ID NO:142 and a light chain variable domain related to SEQ ID NO:143. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:142, and/or incorporate amino acid sequences identical to the CDR1 (SEQ ID NO:144), CDR2 (SEQ ID NO:145), and CDR3 (SEQ ID NO:146) sequences of SEQ ID NO:142 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:143, and/or incorporate amino acid sequences identical to the CDR1 (SEQ ID NO:147), CDR2 (SEQ ID NO:148), and CDR3 (SEQ ID NO:149) sequences of SEQ ID NO:143.

In some embodiments, the second antigen-binding site can bind to CD25 and can incorporate a heavy chain variable domain related to SEQ ID NO:150 and a light chain variable domain related to SEQ ID NO:151. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:150, and/or incorporate amino acid sequences identical to the CDR1 (SEQ ID NO:152), CDR2 (SEQ ID NO:153), and CDR3 (SEQ ID NO:154) sequences of SEQ ID NO:150 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:151, and/or incorporate amino acid sequences identical to the CDR1 (SEQ ID NO:155), CDR2 (SEQ ID NO:156), and CDR3 (SEQ ID NO:157) sequences of SEQ ID NO:151.

In some embodiments, the second antigen-binding site can bind to VLA4 and can incorporate a heavy chain variable domain related to SEQ ID NO:166 and a light chain variable domain related to SEQ ID NO:167. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:166, and/or incorporate amino acid sequences identical to the CDR1 (SEQ ID NO:168), CDR2 (SEQ ID NO:169), and CDR3 (SEQ ID NO:170) sequences of SEQ ID NO:166 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:167, and/or incorporate amino acid sequences identical to the CDR1 (SEQ ID NO:171), CDR2 (SEQ ID NO:172), and CDR3 (SEQ ID NO:173) sequences of SEQ ID NO:167.

In some embodiments, the second antigen-binding site can bind to CD44 and can incorporate a heavy chain variable domain related to SEQ ID NO:174 and a light chain variable domain related to SEQ ID NO:175. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:174, and/or incorporate amino acid sequences identical to the CDR1 (SEQ ID NO:176), CDR2 (SEQ ID NO:177), and CDR3 (SEQ ID NO:178) sequences of SEQ ID NO:174 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:175, and/or incorporate amino acid sequences identical to the CDR1 (SEQ ID NO:179), CDR2 (SEQ ID NO:180), and CDR3 (SEQ ID NO:181) sequences of SEQ ID NO:175.

In some embodiments, the second antigen-binding site can bind to CD47 and can incorporate a heavy chain variable domain related to SEQ ID NO:182 and a light chain variable domain related to SEQ ID NO:183. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:182, and/or incorporate amino acid sequences identical to the CDR1 (SEQ ID NO:184), CDR2 (SEQ ID NO:185), and CDR3 (SEQ ID NO:186) sequences of SEQ ID NO:182 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:183, and/or incorporate amino acid sequences identical to the CDR1 (SEQ ID NO:187), CDR2 (SEQ ID NO:188), and CDR3 (SEQ ID NO:189) sequences of SEQ ID NO:183.

In some embodiments, the second antigen-binding site can bind to CD23 and can incorporate a heavy chain variable domain related to SEQ ID NO:197 and a light chain variable domain related to SEQ ID NO:198. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:197, and/or incorporate amino acid sequences identical to the CDR1 (SEQ ID NO:199), CDR2 (SEQ ID NO:200), and CDR3 (SEQ ID NO:201) sequences of SEQ ID NO:197 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:198, and/or incorporate amino acid sequences identical to the CDR1 (SEQ ID NO:202), CDR2 (SEQ ID NO:203), and CDR3 (SEQ ID NO:204) sequences of SEQ ID NO:198.

In some embodiments, the second antigen-binding site can bind to CD40 and can incorporate a heavy chain variable domain related to SEQ ID NO:205 and a light chain variable domain related to SEQ ID NO:206. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:205, and/or incorporate amino acid sequences identical to the CDR1 (SEQ ID NO:207), CDR2 (SEQ ID NO:208), and CDR3 (SEQ ID NO:209) sequences of SEQ ID NO:205 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:206, and/or incorporate amino acid sequences identical to the CDR1 (SEQ ID NO:210), CDR2 (SEQ ID NO:211), and CDR3 (SEQ ID NO:212) sequences of SEQ ID NO:206.

In some embodiments, the second antigen-binding site can bind to CD40 and can incorporate a heavy chain variable domain related to SEQ ID NO:213 and a light chain variable domain related to SEQ ID NO:214. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:213, and/or incorporate amino acid sequences identical to the CDR1 (SEQ ID NO:215), CDR2 (SEQ ID NO:216), and CDR3 (SEQ ID NO:217) sequences of SEQ ID NO:213 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:214, and/or incorporate amino acid sequences identical to the CDR1 (SEQ ID NO:218), CDR2 (SEQ ID NO:219), and CDR3 (SEQ ID NO:220) sequences of SEQ ID NO:214.

In some embodiments, the second antigen-binding site can bind to CD40 and can incorporate a heavy chain variable domain related to SEQ ID NO:221 and a light chain variable domain related to SEQ ID NO:222. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:221, and/or incorporate amino acid sequences identical to the CDR1 (SEQ ID NO:223), CDR2 (SEQ ID NO:224), and CDR3 (SEQ ID NO:225) sequences of SEQ ID NO:221 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:222, and/or incorporate amino acid sequences identical to the CDR1 (SEQ ID NO:226), CDR2 (SEQ ID NO:227), and CDR3 (SEQ ID NO:228) sequences of SEQ ID NO:222.

In some embodiments, the second antigen-binding site can bind to CD40 and can incorporate a heavy chain variable domain related to SEQ ID NO:229 and a light chain variable domain related to SEQ ID NO:230. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:229, and/or incorporate amino acid sequences identical to the CDR1 (SEQ ID NO:231), CDR2 (SEQ ID NO:232), and CDR3 (SEQ ID NO:233) sequences of SEQ ID NO:229 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:230, and/or incorporate amino acid sequences identical to the CDR1 (SEQ ID NO:234), CDR2 (SEQ ID NO:235), and CDR3 (SEQ ID NO:236) sequences of SEQ ID NO:230.

In some embodiments, the second antigen-binding site can bind to CD70 and can incorporate a heavy chain variable domain related to SEQ ID NO:237 and a light chain variable domain related to SEQ ID NO:238. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:237, and/or incorporate amino acid sequences identical to the CDR1 (SEQ ID NO:239), CDR2 (SEQ ID NO:240), and CDR3 (SEQ ID NO:241) sequences of SEQ ID NO:237 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:238, and/or incorporate amino acid sequences identical to the CDR1 (SEQ ID NO:242), CDR2 (SEQ ID NO:243), and CDR3 (SEQ ID NO:244) sequences of SEQ ID NO:238.

In some embodiments, the second antigen-binding site can bind to CD79b and can incorporate a heavy chain variable domain related to SEQ ID NO:245 and a light chain variable domain related to SEQ ID NO:246. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:245, and/or incorporate amino acid sequences identical to the CDR1 (SEQ ID NO:247), CDR2 (SEQ ID NO:248), and CDR3 (SEQ ID NO:249) sequences of SEQ ID NO:245 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:246, and/or incorporate amino acid sequences identical to the CDR1 (SEQ ID NO:250), CDR2 (SEQ ID NO:251), and CDR3 (SEQ ID NO:252) sequences of SEQ ID NO:246.

In some embodiments, the second antigen-binding site can bind to CD80 and can incorporate a heavy chain variable domain related to SEQ ID NO:253 and a light chain variable domain related to SEQ ID NO:254. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:253, and/or incorporate amino acid sequences identical to the CDR1 (SEQ ID NO:255), CDR2 (SEQ ID NO:256), and CDR3 (SEQ ID NO:257) sequences of SEQ ID NO:253 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:254, and/or incorporate amino acid sequences identical to the CDR1 (SEQ ID NO:258), CDR2 (SEQ ID NO:259), and CDR3 (SEQ ID NO:260) sequences of SEQ ID NO:254.

In some embodiments, the second antigen-binding site can bind to CRLF2 and can incorporate a heavy chain variable domain related to SEQ ID NO:261 and a light chain variable domain related to SEQ ID NO:262. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:261, and/or incorporate amino acid sequences identical to the CDR1 (SEQ ID NO:263), CDR2 (SEQ ID NO:264), and CDR3 (SEQ ID NO:265) sequences of SEQ ID NO:261 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:262, and/or incorporate amino acid sequences identical to the CDR1 (SEQ ID NO:266), CDR2 (SEQ ID NO:267), and CDR3 (SEQ ID NO:268) sequences of SEQ ID NO:262.

In some embodiments, the second antigen-binding site can bind to SLAMF7 and can incorporate a heavy chain variable domain related to SEQ ID NO:272 and a light chain variable domain related to SEQ ID NO:273. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:272, and/or incorporate amino acid sequences identical to the CDR1 (SEQ ID NO:274), CDR2 (SEQ ID NO:275), and CDR3 (SEQ ID NO:276) sequences of SEQ ID NO:272 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:273, and/or incorporate amino acid sequences identical to the CDR1 (SEQ ID NO:277), CDR2 (SEQ ID NO:278), and CDR3 (SEQ ID NO:279) sequences of SEQ ID NO:273.

In some embodiments, the second antigen-binding site can bind to SLAMF7 and can incorporate a heavy chain variable domain related to SEQ ID NO:280 and a light chain variable domain related to SEQ ID NO:281. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:280, and/or incorporate amino acid sequences identical to the CDR1 (SEQ ID NO:282), CDR2 (SEQ ID NO:283), and CDR3 (SEQ ID NO:284) sequences of SEQ ID NO:280 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:281, and/or incorporate amino acid sequences identical to the CDR1 (SEQ ID NO:285), CDR2 (SEQ ID NO:286), and CDR3 (SEQ ID NO:287) sequences of SEQ ID NO:281.

In some embodiments, the second antigen-binding site can bind to CD138 and can incorporate a heavy chain variable domain related to SEQ ID NO:288 and a light chain variable domain related to SEQ ID NO:289. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:288, and/or incorporate amino acid sequences identical to the CDR1 (SEQ ID NO:290), CDR2 (SEQ ID NO:291), and CDR3 (SEQ ID NO:292) sequences of SEQ ID NO:288 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:289, and/or incorporate amino acid sequences identical to the CDR1 (SEQ ID NO:293), CDR2 (SEQ ID NO:294), and CDR3 (SEQ ID NO:295) sequences of SEQ ID NO:289.

In some embodiments, the second antigen-binding site can bind to CD38 and can incorporate a heavy chain variable domain related to SEQ ID NO:296 and a light chain variable domain related to SEQ ID NO:297. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:296, and/or incorporate amino acid sequences identical to the CDR1 (SEQ ID NO:298), CDR2 (SEQ ID NO:299), and CDR3 (SEQ ID NO:300) sequences of SEQ ID NO:296 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:297, and/or incorporate amino acid sequences identical to the CDR1 (SEQ ID NO:301), CDR2 (SEQ ID NO:302), and CDR3 (SEQ ID NO:303) sequences of SEQ ID NO:297.

In some embodiments, the second antigen-binding site can bind to CD38 and can incorporate a heavy chain variable domain related to SEQ ID NO:304 and a light chain variable domain related to SEQ ID NO:305. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:304, and/or incorporate amino acid sequences identical to the CDR1 (SEQ ID NO:306), CDR2 (SEQ ID NO:307), and CDR3 (SEQ ID NO:308) sequences of SEQ ID NO:304 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:305, and/or incorporate amino acid sequences identical to the CDR1 (SEQ ID NO:309), CDR2 (SEQ ID NO:310), and CDR3 (SEQ ID NO:311) sequences of SEQ ID NO:305.

In some embodiments, the second antigen-binding site can bind to CD7 and can incorporate a heavy chain variable domain related to SEQ ID NO:325. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:325, and/or incorporate amino acid sequences identical to the CDR1 (SEQ ID NO:326), CDR2 (SEQ ID NO:327), and CDR3 (SEQ ID NO:328) sequences of SEQ ID NO:325.

In some embodiments, the second antigen-binding site can bind to CD7 and can incorporate a heavy chain variable domain related to SEQ ID NO:329. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:329, and/or incorporate amino acid sequences identical to the CDR1 (SEQ ID NO:330), CDR2 (SEQ ID NO:331), and CDR3 (SEQ ID NO:332) sequences of SEQ ID NO:329.

In some embodiments, the second antigen-binding site can bind to CTLA4 and can incorporate a heavy chain variable domain related to SEQ ID NO:333 and a light chain variable domain related to SEQ ID NO:334. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:333, and/or incorporate amino acid sequences identical to the CDR1 (SEQ ID NO:335), CDR2 (SEQ ID NO:336), and CDR3 (SEQ ID NO:337) sequences of SEQ ID NO:333 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:334, and/or incorporate amino acid sequences identical to the CDR1 (SEQ ID NO:338), CDR2 (SEQ ID NO:339), and CDR3 (SEQ ID NO:340) sequences of SEQ ID NO:334.

In some embodiments, the second antigen-binding site can bind to CTLA4 and can incorporate a heavy chain variable domain related to SEQ ID NO:341 and a light chain variable domain related to SEQ ID NO:342. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:341, and/or incorporate amino acid sequences identical to the CDR1 (SEQ ID NO:343), CDR2 (SEQ ID NO:344), and CDR3 (SEQ ID NO:345) sequences of SEQ ID NO:341 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:342, and/or incorporate amino acid sequences identical to the CDR1 (SEQ ID NO:346), CDR2 (SEQ ID NO:347), and CDR3 (SEQ ID NO:348) sequences of SEQ ID NO:342.

In some embodiments, the second antigen-binding site can bind to CX3CR1 and can incorporate a heavy chain variable domain related to SEQ ID NO:349. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:349, and/or incorporate amino acid sequences identical to the CDR1 (SEQ ID NO:350), CDR2 (SEQ ID NO:351), and CDR3 (SEQ ID NO:352) sequences of SEQ ID NO:349.

In some embodiments, the second antigen-binding site can bind to CX3CR1 and can incorporate a heavy chain variable domain related to SEQ ID NO:353. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:353, and/or incorporate amino acid sequences identical to the CDR1 (SEQ ID NO:354), CDR2 (SEQ ID NO:356), and CDR3 (SEQ ID NO:357) sequences of SEQ ID NO:353.

In some embodiments, the second antigen-binding site can bind to ENTPD1 and can incorporate a heavy chain variable domain related to SEQ ID NO:358 and a light chain variable domain related to SEQ ID NO:359. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:358, and/or incorporate amino acid sequences identical to the CDR1 (SEQ ID NO:360), CDR2 (SEQ ID NO:361), and CDR3 (SEQ ID NO:362) sequences of SEQ ID NO:358 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:359, and/or incorporate amino acid sequences identical to the CDR1 (SEQ ID NO:363), CDR2 (SEQ ID NO:364), and CDR3 (SEQ ID NO:365) sequences of SEQ ID NO:359.

In some embodiments, the second antigen-binding site can bind to ENTPD1 and can incorporate a heavy chain variable domain related to SEQ ID NO:366 and a light chain variable domain related to SEQ ID NO:367. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:366, and/or incorporate amino acid sequences identical to the CDR1 (SEQ ID NO:368), CDR2 (SEQ ID NO:369), and CDR3 (SEQ ID NO:370) sequences of SEQ ID NO:366 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:367, and/or incorporate amino acid sequences identical to the CDR1 (SEQ ID NO:371), CDR2 (SEQ ID NO:372), and CDR3 (SEQ ID NO:373) sequences of SEQ ID NO:367.

In some embodiments, the second antigen-binding site can bind to HAVCR2 and can incorporate a heavy chain variable domain related to SEQ ID NO:374 and a light chain variable domain related to SEQ ID NO:375. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:374, and/or incorporate amino acid sequences identical to the CDR1 (SEQ ID NO:376), CDR2 (SEQ ID NO:377), and CDR3 (SEQ ID NO:378) sequences of SEQ ID NO:374 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:375, and/or incorporate amino acid sequences identical to the CDR1 (SEQ ID NO:379), CDR2 (SEQ ID NO:380), and CDR3 (SEQ ID NO:381) sequences of SEQ ID NO:375.

In some embodiments, the second antigen-binding site can bind to HAVCR2 and can incorporate a heavy chain variable domain related to SEQ ID NO:382 and a light chain variable domain related to SEQ ID NO:383. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:382, and/or incorporate amino acid sequences identical to the CDR1 (SEQ ID NO:384), CDR2 (SEQ ID NO:385), and CDR3 (SEQ ID NO:386) sequences of SEQ ID NO:382 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:383, and/or incorporate amino acid sequences identical to the CDR1 (SEQ ID NO:387), CDR2 (SEQ ID NO:388), and CDR3 (SEQ ID NO:389) sequences of SEQ ID NO:383.

In some embodiments, the second antigen-binding site can bind to PDCDILG2 and can incorporate a heavy chain variable domain related to SEQ ID NO:390 and a light chain variable domain related to SEQ ID NO:391. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:390, and/or incorporate amino acid sequences identical to the CDR1 (SEQ ID NO:392), CDR2 (SEQ ID NO:393), and CDR3 (SEQ ID NO:394) sequences of SEQ ID NO:390. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:391, and/or incorporate amino acid sequences identical to the CDR1 (SEQ ID NO:395), CDR2 (SEQ ID NO:396), and CDR3 (SEQ ID NO:397) sequences of SEQ ID NO:391.

In some embodiments, the second antigen-binding site can bind to PDCDILG2 and can incorporate a heavy chain variable domain related to SEQ ID NO:398 and a light chain variable domain related to SEQ ID NO:399. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:398, and/or incorporate amino acid sequences identical to the CDR1 (SEQ ID NO:400), CDR2 (SEQ ID NO:401), and CDR3 (SEQ ID NO:402) sequences of SEQ ID NO:398. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:399, and/or incorporate amino acid sequences identical to the CDR1 (SEQ ID NO:403), CDR2 (SEQ ID NO:404), and CDR3 (SEQ ID NO:405) sequences of SEQ ID NO:399.

In some embodiments, the second antigen-binding site can bind to TIGIT and can incorporate a heavy chain variable domain related to SEQ ID NO:406 and a light chain variable domain related to SEQ ID NO:407. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:406, and/or incorporate amino acid sequences identical to the CDR1 (SEQ ID NO:408), CDR2 (SEQ ID NO:409), and CDR3 (SEQ ID NO:410) sequences of SEQ ID NO:406 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:407, and/or incorporate amino acid sequences identical to the CDR1 (SEQ ID NO:411), CDR2 (SEQ ID NO:412), and CDR3 (SEQ ID NO:413) sequences of SEQ ID NO:407.

In some embodiments, the second antigen-binding site can bind to TIGIT and can incorporate a heavy chain variable domain related to SEQ ID NO:414 and a light chain variable domain related to SEQ ID NO:415. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:414, and/or incorporate amino acid sequences identical to the CDR1 (SEQ ID NO:416), CDR2 (SEQ ID NO:417), and CDR3 (SEQ ID NO:418) sequences of SEQ ID NO:414 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:415, and/or incorporate amino acid sequences identical to the CDR1 (SEQ ID NO:419), CDR2 (SEQ ID NO:420), and CDR3 (SEQ ID NO:421) sequences of SEQ ID NO:415.

In some embodiments, the second antigen-binding site can bind to TNFRSF4 and can incorporate a heavy chain variable domain related to SEQ ID NO:422 and a light chain variable domain related to SEQ ID NO:423. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:422, and/or incorporate amino acid sequences identical to the CDR1 (SEQ ID NO:424), CDR2 (SEQ ID NO:425), and CDR3 (SEQ ID NO:426) sequences of SEQ ID NO:422 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:423, and/or incorporate amino acid sequences identical to the CDR1 (SEQ ID NO:427), CDR2 (SEQ ID NO:428), and CDR3 (SEQ ID NO:429) sequences of SEQ ID NO:423.

In some embodiments, the second antigen-binding site can bind to TNFRSF4 and can incorporate a heavy chain variable domain related to SEQ ID NO:430 and a light chain variable domain related to SEQ ID NO:431. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:430, and/or incorporate amino acid sequences identical to the CDR1 (SEQ ID NO:432), CDR2 (SEQ ID NO:433), and CDR3 (SEQ ID NO:434) sequences of SEQ ID NO:430 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:431, and/or incorporate amino acid sequences identical to the CDR1 (SEQ ID NO:435), CDR2 (SEQ ID NO:436), and CDR3 (SEQ ID NO:437) sequences of SEQ ID NO:431.

In some embodiments, the second antigen-binding site can bind to TNFRSF8 and can incorporate a heavy chain variable domain related to SEQ ID NO:438 and a light chain variable domain related to SEQ ID NO:439. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:438, and/or incorporate amino acid sequences identical to the CDR1 (SEQ ID NO:440), CDR2 (SEQ ID NO:441), and CDR3 (SEQ ID NO:442) sequences of SEQ ID NO:438 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:439, and/or incorporate amino acid sequences identical to the CDR1 (SEQ ID NO:443), CDR2 (SEQ ID NO:444), and CDR3 (SEQ ID NO:445) sequences of SEQ ID NO:439.

In some embodiments, the second antigen-binding site can bind to TNFRSF8 and can incorporate a heavy chain variable domain related to SEQ ID NO:446 and a light chain variable domain related to SEQ ID NO:447. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:446, and/or incorporate amino acid sequences identical to the CDR1 (SEQ ID NO:448), CDR2 (SEQ ID NO:449), and CDR3 (SEQ ID NO:450) sequences of SEQ ID NO:446 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:447, and/or incorporate amino acid sequences identical to the CDR1 (SEQ ID NO:451), CDR2 (SEQ ID NO:452), and CDR3 (SEQ ID NO:453) sequences of SEQ ID NO:447.

In some embodiments, the second antigen-binding site can bind to TNFRSF9 and can incorporate a heavy chain variable domain related to SEQ ID NO:454 and a light chain variable domain related to SEQ ID NO:455. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:454, and/or incorporate amino acid sequences identical to the CDR1 (SEQ ID NO:456), CDR2 (SEQ ID NO:457), and CDR3 (SEQ ID NO:458) sequences of SEQ ID NO:454 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:455, and/or incorporate amino acid sequences identical to the CDR1 (SEQ ID NO:459), CDR2 (SEQ ID NO:460), and CDR3 (SEQ ID NO:461) sequences of SEQ ID NO:455.

In some embodiments, the second antigen-binding site can bind to TNFRSF9 and can incorporate a heavy chain variable domain related to SEQ ID NO:462 and a light chain variable domain related to SEQ ID NO:463. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:462, and/or incorporate amino acid sequences identical to the CDR1 (SEQ ID NO:464), CDR2 (SEQ ID NO:465), and CDR3 (SEQ ID NO:466) sequences of SEQ ID NO:462 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:463, and/or incorporate amino acid sequences identical to the CDR1 (SEQ ID NO:467), CDR2 (SEQ ID NO:468), and CDR3 (SEQ ID NO:469) sequences of SEQ ID NO:463.

In some embodiments, the second antigen-binding site can bind to NST5 and can incorporate a heavy chain variable domain related to SEQ ID NO:470 and a light chain variable domain related to SEQ ID NO:471. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:470, and/or incorporate amino acid sequences identical to the CDR1 (SEQ ID NO:472), CDR2 (SEQ ID NO:473), and CDR3 (SEQ ID NO:474) sequences of SEQ ID NO:470 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:471, and/or incorporate amino acid sequences identical to the CDR1 (SEQ ID NO:475), CDR2 (SEQ ID NO:476), and CDR3 (SEQ ID NO:477) sequences of SEQ ID NO:471.

In some embodiments, the second antigen-binding site can bind to NST5 and can incorporate a heavy chain variable domain related to SEQ ID NO:478 and a light chain variable domain related to SEQ ID NO:479. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:478, and/or incorporate amino acid sequences identical to the CDR1 (SEQ ID NO:480), CDR2 (SEQ ID NO:481), and CDR3 (SEQ ID NO:482) sequences of SEQ ID NO:478 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:479, and/or incorporate amino acid sequences identical to the CDR1 (SEQ ID NO:483), CDR2 (SEQ ID NO:484), and CDR3 (SEQ ID NO:485) sequences of SEQ ID NO:479.

In some embodiments, the second antigen-binding site can bind to TNFRSF18 and can incorporate a heavy chain variable domain related to SEQ ID NO:486 and a light chain variable domain related to SEQ ID NO:487. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:486, and/or incorporate amino acid sequences identical to the CDR1 (SEQ ID NO:488), CDR2 (SEQ ID NO:489), and CDR3 (SEQ ID NO:490) sequences of SEQ ID NO:486 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:487, and/or incorporate amino acid sequences identical to the CDR1 (SEQ ID NO:491), CDR2 (SEQ ID NO:492), and CDR3 (SEQ ID NO:493) sequences of SEQ ID NO:487.

In some embodiments, the second antigen-binding site can bind to TNFRSF18 and can incorporate a heavy chain variable domain related to SEQ ID NO:494 and a light chain variable domain related to SEQ ID NO:495. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:494, and/or incorporate amino acid sequences identical to the CDR1 (SEQ ID NO:496), CDR2 (SEQ ID NO:497), and CDR3 (SEQ ID NO:498) sequences of SEQ ID NO:494 Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:495, and/or incorporate amino acid sequences identical to the CDR1 (SEQ ID NO:499), CDR2 (SEQ ID NO:500), and CDR3 (SEQ ID NO:501) sequences of SEQ ID NO:495.

In some embodiments, the second antigen binding site incorporates a light chain variable domain having an amino acid sequence identical to the amino acid sequence of the light chain variable domain present in the first antigen binding site.

In some embodiments, the protein incorporates a portion of an antibody Fc domain sufficient to bind CD16, wherein the antibody Fc domain comprises hinge and CH2 domains, and/or amino acid sequences at least 90% identical to amino acid sequence 234-332 of a human IgG antibody.

Formulations containing one of these proteins; cells containing one or more nucleic acids expressing these proteins, and methods of enhancing tumor cell death using these proteins are also provided.

Another aspect of the invention provides a method of treating cancer in a patient. The method comprises administering to a patient in need thereof a therapeutically effective amount of the multi-specific binding protein described herein. Exemplary cancers for treatment using the multi-specific binding proteins include, for example, acute myeloid leukemia, diffuse large B cell lymphoma, thymoma, adenoid cystic carcinoma, gastrointestinal cancer, renal cancer, breast cancer, glioblastoma, lung cancer, ovarian cancer, brain cancer, prostate cancer, pancreatic cancer, and melanomas.

### BRIEF DESCRIPTION OF THE DRAWINGS

FIG. 1 is a representation of a heterodimeric, multi-specific antibody (a trispecific binding protein (TriNKET)). Each arm can represent either the NKG2D-binding domain, or the tumor associated antigen-binding domain. In some embodiments, the NKG2D- and the tumor associated antigen-binding domains can share a common light chain.

FIG. 2 is a representation of a heterodimeric, multi-specific antibody. Either the NKG2D-binding domain or the tumor associated antigen-binding domain can take the scFv format (right arm).

FIG. 3 are line graphs demonstrating the binding affinity of NKG2D-binding domains (listed as clones) to human recombinant NKG2D in an ELISA assay.

FIG. 4 are line graphs demonstrating the binding affinity of NKG2D-binding domains (listed as clones) to cynomolgus recombinant NKG2D in an ELISA assay.

FIG. 5 are line graphs demonstrating the binding affinity of NKG2D-binding domains (listed as clones) to mouse recombinant NKG2D in an ELISA assay.

FIG. 6 are bar graphs demonstrating the binding of NKG2D-binding domains (listed as clones) to EL4 cells expressing human NKG2D by flow cytometry showing mean fluorescence intensity (MFI) fold over background (FOB).

FIG. 7 are bar graphs demonstrating the binding of NKG2D-binding domains (listed as clones) to EL4 cells expressing mouse NKG2D by flow cytometry showing mean fluorescence intensity (MFI) fold over background (FOB).

FIG. 8 are line graphs demonstrating specific binding affinity of NKG2D-binding domains (listed as clones) to recombinant human NKG2D-Fc by competing with natural ligand ULBP-6.

FIG. 9 are line graphs demonstrating specific binding affinity of NKG2D-binding domains (listed as clones) to recombinant human NKG2D-Fc by competing with natural ligand MICA.

FIG. 10 are line graphs demonstrating specific binding affinity of NKG2D-binding domains (listed as clones) to recombinant mouse NKG2D-Fc by competing with natural ligand Rae-1 delta.

FIG. 11 are bar graphs showing activation of human NKG2D by NKG2D-binding domains (listed as clones) by quantifying the percentage of TNF-α positive cells, which express human NKG2D-CD3 zeta fusion proteins.

FIG. 12 are bar graphs showing activation of mouse NKG2D by NKG2D-binding domains (listed as clones) by quantifying the percentage of TNF-α positive cells, which express mouse NKG2D-CD3 zeta fusion proteins.

FIG. 13 are bar graphs showing activation of human NK cells by NKG2D-binding domains (listed as clones).

FIG. 14 are bar graphs showing activation of human NK cells by NKG2D-binding domains (listed as clones).

FIG. 15 are bar graphs showing activation of mouse NK cells by NKG2D-binding domains (listed as clones).

FIG. 16 are bar graphs showing activation of mouse NK cells by NKG2D-binding domains (listed as clones).

FIG. 17 are bar graphs showing the cytotoxic effect of NKG2D-binding domains (listed as clones) on tumor cells.

FIG. 18 are bar graphs showing the melting temperature of NKG2D-binding domains (listed as clones) measured by differential scanning fluorimetry.

FIGS. 19A-19C are bar graphs of synergistic activation of NK cells using CD16 and NKG2D-binding. FIG. 19A demonstrates levels of CD107a; FIG. 19B demonstrates levels of IFN-γ; FIG. 19C demonstrates levels of CD107a and IFN-γ. Graphs indicate the mean (n=2) ±SD. Data are representative of five independent experiments using five different healthy donors.

FIG. 20 is a representation of a trispecific binding protein (TriNKET) in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 21 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 22 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. DVD-Ig™ form contains normal Fc.

FIG. 23 is a representation of a TriNKET in the Orthogonal Fab interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 24 is a representation of a TriNKET in the 2-in-1 Ig format.

FIG. 25 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 26 is a representation of a TriNKET in the Fab fragment Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 27 is a representation of a TriNKET in the SEED Body form, which is a heterodimer containing 2 Fab fragments binding to target 1 and 2, and an Fc stabilized by heterodimerization mutations.

FIG. 28 is a representation of a TriNKET in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 29 is a representation of a TriNKET in the Cov-X-Body form.

FIGS. 30A and 30B are representations of TriNKETs in the κλ-Body forms, which are heterodimeric constructs with two different Fab fragments fused to Fc stabilized by heterodimerization mutations: one Fab fragment targeting antigen 1 contains kappa LC, and the second Fab fragment targeting antigen 2 contains lambda LC. FIG. 30A is an exemplary representation of one form of a κλ-Body; FIG. 30B is an exemplary representation of another κλ-Body.

FIG. 31 is an Oasc-Fab heterodimeric construct that includes Fab fragment binding to target 1 and scFab binding to target 2, both of which are fused to the Fc domain. Heterodimerization is ensured by mutations in the Fc domain.

FIG. 32 is a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 33 is a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, while CL is fused in-line with VH.

FIG. 34 is a Fit-Ig, which is a homodimeric construct where Fab fragment binding to antigen 2 is fused to the N-terminus of HC of Fab fragment that binds to antigen 1. The construct contains wild-type Fc.

FIG. 35 shows data from a FACS showing expression of CXCR4 on human B cell lymphoma cell line Raji (Black=Isotype control; Empty=CXCR4 staining).

FIG. 36 are line graphs showing that CXCR4-TriNKETs mediate KHYG-1 killing of Raji target cells.

FIG. 37 is a bar graph showing that CXCR4-targeted TrINKETs mediate human NK cell killing of Raji target cells.

### DETAILED DESCRIPTION

The invention provides multi-specific binding proteins that bind CXCR4 on a cancer cell and the NKG2D receptor and CD16 receptor on natural killer cells to activate the natural killer cells, pharmaceutical compositions comprising such multi-specific binding proteins, and therapeutic methods using such multi-specific proteins and pharmaceutical compositions, including for the treatment of cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR”. Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor associated antigen” as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind to the NKG2D receptor and CD16 receptor on natural killer cells, and the tumor-associated antigen selected from any one of the antigens provided in Table 15. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing the tumor-associated antigen selected from any one of the antigens provided in Table 15. Binding of the multi-specific binding proteins to tumor-associated antigen-expressing cells brings the cancer cells into proximity with the natural killer cell, which facilitates direct and indirect destruction of the cancer cells by the natural killer cell. Further description of some exemplary multi-specific binding proteins is provided below.

The first component of the multi-specific binding proteins binds to NKG2D receptor-expressing cells, which can include but are not limited to NK cells, γδ T cells and CD8⁺αβ T cells. Upon NKG2D binding, the multi-specific binding proteins may block natural ligands, such as ULBP6 (UL16 binding protein 6) and MICA (Major Histocompatibility Complex Class I Chain-Related A), from binding to NKG2D and activating NKG2D receptors.

The second component of the multi-specific binding proteins binds a tumor-associated antigen selected from any one of the antigens provided in Table 15. The tumor-associated antigen-expressing cells, which may be found in leukemias such as, for example, acute myeloid leukemia and T-cell leukemia.

The third component for the multi-specific binding proteins binds to cells expressing CD16, an Fc receptor on the surface of leukocytes including natural killer cells, macrophages, neutrophils, eosinophils, mast cells, and follicular dendritic cells.

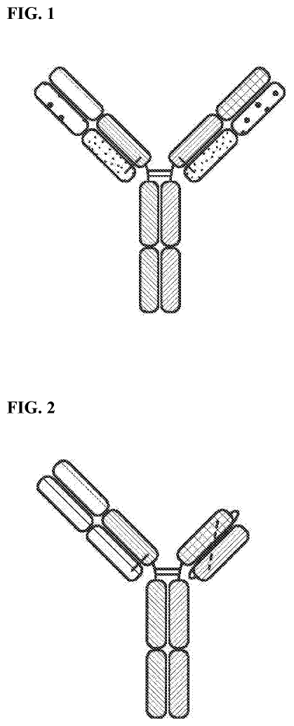

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. The second immunoglobulin light chain, together with the second immunoglobulin heavy chain, forms an antigen-binding site that binds a tumor-associated antigen selected from any one of the antigens provided in Table 15. The first Fc domain and second Fc domain together are able to bind to CD16 (FIG. 1). In some embodiments, the first immunoglobulin light chain is identical to the second immunoglobulin light chain.

Another exemplary format involves a heterodimeric, multi-specific antibody including a first immunoglobulin heavy chain, a second immunoglobulin heavy chain and an immunoglobulin light chain (FIG. 2). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind a tumor-associated antigen selected from any one of the antigens provided in Table 15. The second immunoglobulin heavy chain includes a second Fc (hinge-CH2-CH3) domain, a second heavy chain variable domain and optionally a CH1 heavy chain domain. The immunoglobulin light chain includes a light chain variable domain and a light chain constant domain. The second immunoglobulin heavy chain pairs with the immunoglobulin light chain and binds to NKG2D or binds a tumor-associated antigen selected from any one of the antigens provided in Table 15. The first Fc domain and the second Fc domain together are able to bind to CD16 (FIG. 2).

One or more additional binding motifs may be fused to the C-terminus of the constant region CH3 domain, optionally via a linker sequence. In certain embodiments, the antigen-binding motif is a single-chain or disulfide-stabilized variable region (scFv) forming a tetravalent or trivalent molecule.

In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multi-specific binding protein is in the 2-in-1 Ig format. In some embodiments, the multi-specific binding protein is in the ES form, which is a heterodimeric construct containing two different Fab fragments binding to targets 1 and target 2 fused to the Fc. Heterodimerization is ensured by electrostatic steering mutations in the Fc.

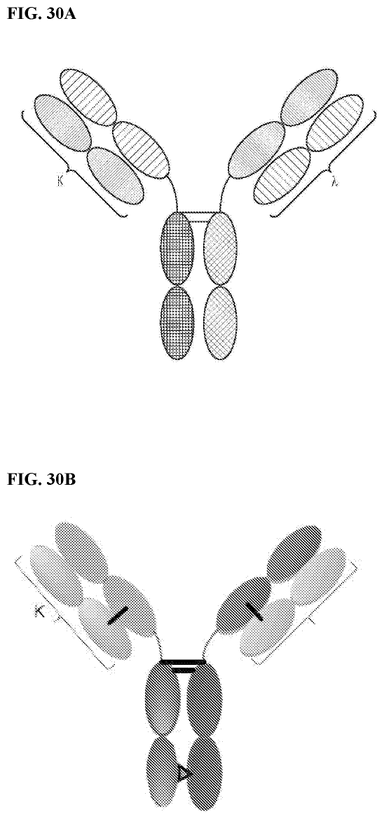

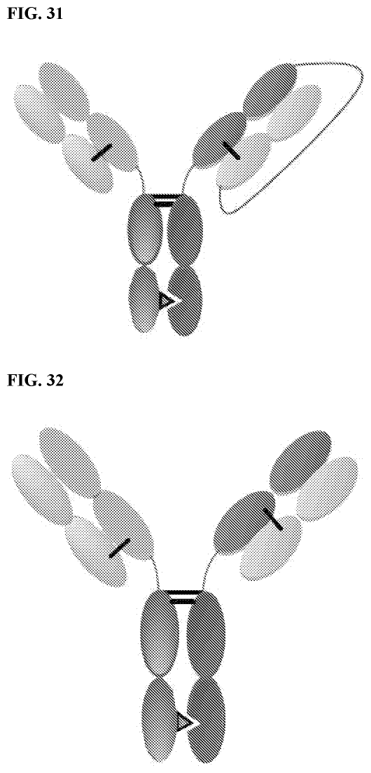

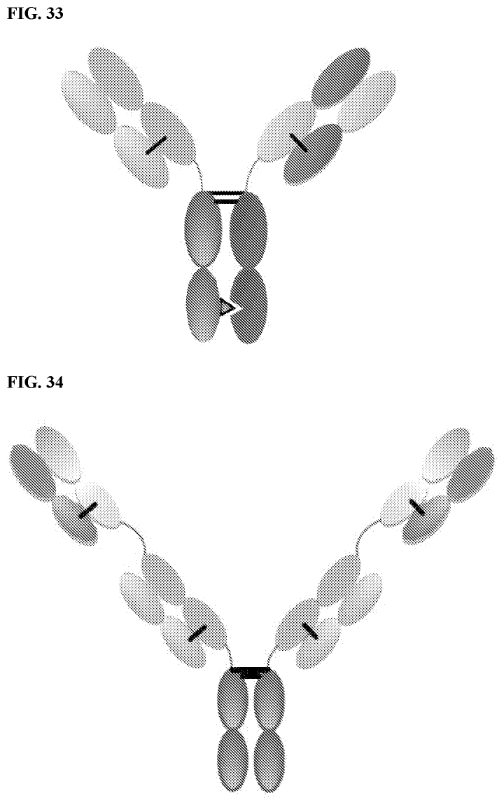

In some embodiments, the multi-specific binding protein is in the κλ-Body form, which is a heterodimeric construct with two different Fab fragments fused to Fc stabilized by heterodimerization mutations: Fab fragment 1 targeting antigen 1 contains kappa LC, while second Fab fragment targeting antigen 2 contains lambda LC. FIG. 30A is an exemplary representation of one form of a κλ-Body; FIG. 30B is an exemplary representation of another κλ-Body.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies).

In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which a leucine zipper is used to induce heterodimerization of two different HCs. (Wranik, BJ. et al., J. Biol. Chem. (2012), 287:43331-9).

In some embodiments, the multi-specific binding protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab fragment binding to target 1, and scFab binding to target 2 fused to Fc. Heterodimerization is ensured by mutations in the Fc.

In some embodiments, the multi-specific binding protein is in a DuetMab form, which is a heterodimeric construct containing two different Fab fragments binding to antigens 1 and 2, and Fc stabilized by heterodimerization mutations. Fab fragments 1 and 2 contain differential S-S bridges that ensure correct LC and HC pairing.

In some embodiments, the multi-specific binding protein is in a CrossmAb form, which is a heterodimeric construct with two different Fab fragments binding to targets 1 and 2, fused to Fc stabilized by heterodimerization. CL and CH1 domains and VH and VL domains are switched, e.g., CH1 is fused in-line with VL, while CL is fused in-line with VH.

In some embodiments, the multi-specific binding protein is in a Fit-Ig form, which is a homodimeric construct where Fab fragment binding to antigen 2 is fused to the N terminus of HC of Fab fragment that binds to antigen 1. The construct contains wild-type Fc.

Table 1 lists peptide sequences of heavy chain variable domains and light chain variable domains that, in combination, can bind to NKG2D. The NKG2D binding domains can vary in their binding affinity to NKG2D, nevertheless, they all activate human NKG2D and NK cells.

TABLE 1

| | Heavy chain | Light chain |
|---|---|---|
| | variable region | variable region |
| Clones | amino acid sequence | amino acid sequence |
| ADI- | QVQLQQWGAGLLKPSETLSLTCAV | DIQMTQSPSTLSASVGDRVTITCR |
| 27705 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYNSYPI |
| | RARGPWSFDPWGQGTLVTVSS | TFGGGTKVEIK |
| | (SEQ ID NO: 1) | (SEQ ID NO: 2) |
| | CDR1 (SEQ ID NO: 105)- | |
| | GSFSGYYWS | |
| | CDR2 (SEQ ID NO: 106)- | |
| | EIDHSGSTNYNPSLKS | |
| | CDR3 (SEQ ID NO: 107)- | |
| | ARARGPWSFDP | |
| ADI- | QVQLQQWGAGLLKPSETLSLTCAV | EIVLTQSPGTLSLSPGERATLSCR |
| 27724 | YGGSFSGYYWSWIRQPPGKGLEWI | ASQSVSSSYLAWYQQKPGQAPRLL |
| | GEIDHSGSTNYNPSLKSRVTISVD | IYGASSRATGIPDRFSGSGSGTDF |
| | TSKNQFSLKLSSVTAADTAVYYCA | TLTISRLEPEDFAVYYCQQYGSSP |
| | RARGPWSFDPWGQGTLVTVSS | ITFGGGTKVEIK |
| | (SEQ ID NO: 3) | (SEQ ID NO: 4) |
| ADI- | QVQLQQWGAGLLKPSETLSLTCAV | DIQMTQSPSTLSASVGDRVTITCR |
| 27740 | YGGSFSGYYWSWIRQPPGKGLEWI | ASQSIGSWLAWYQQKPGKAPKLLI |
| (A40) | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYHSFYT |
| | RARGPWSFDPWGQGTLVTVSS | FGGGTKVEIK |
| | (SEQ ID NO: 5) | (SEQ ID NO: 6) |
| ADI- | QVQLQQWGAGLLKPSETLSLTCAV | DIQMTQSPSTLSASVGDRVTITCR |
| 27741 | YGGSFSGYYWSWIRQPPGKGLEWI | ASQSIGSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQSNSYYT |
| | RARGPWSFDPWGQGTLVTVSS | FGGGTKVEIK |
| | (SEQ ID NO: 7) | (SEQ ID NO: 8) |
| ADI- | QVQLQQWGAGLLKPSETLSLTCAV | DIQMTQSPSTLSASVGDRVTITCR |
| 27743 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYNSYPT |
| | RARGPWSFDPWGQGTLVTVSS | FGGGTKVEIK |
| | (SEQ ID NO: 9) | (SEQ ID NO: 10) |
| ADI- | QVQLQQWGAGLLKPSETLSLTCAV | ELQMTQSPSSLSASVGDRVTITCR |
| 28153 | YGGSFSGYYWSWIRQPPGKGLEWI | TSQSISSYLNWYQQKPGQPPKLLI |
| | GEIDHSGSTNYNPSLKSRVTISVD | YWASTRESGVPDRFSGSGSGTDFT |
| | TSKNQFSLKLSSVTAADTAVYYCA | LTISSLQPEDSATYYCQQSYDIPY |
| | RARGPWGFDPWGQGTLVTVSS | TFGQGTKLEIK |
| | (SEQ ID NO: 11) | (SEQ ID NO: 12) |
| ADI- | QVQLQQWGAGLLKPSETLSLTCAV | DIQMTQSPSTLSASVGDRVTITCR |
| 28226 | YGGSFSGYYWSWIRQPPGKGLEWI | ASQSISSWLAWYQQKPGKAPKLLI |
| (C26) | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYGSFPI |
| | RARGPWSFDPWGQGTLVTVSS | TFGGGTKVEIK |
| | (SEQ ID NO: 13) | (SEQ ID NO: 14) |
| ADI- | QVQLQQWGAGLLKPSETLSLTCAV | DIQMTQSPSTLSASVGDRVTITCR |
| 28154 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTDFT |
| | TSKNQFSLKLSSVTAADTAVYYCA | LTISSLQPDDFATYYCQQSKEVPW |
| | RARGPWSFDPWGQGTLVTVSS | TFGQGTKVEIK |
| | (SEQ ID NO: 15) | (SEQ ID NO: 16) |
| ADI- | QVQLQQWGAGLLKPSETLSLTCAV | DIQMTQSPSTLSASVGDRVTITCR |
| 29399 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYNSFPT |
| | RARGPWSFDPWGQGTLVTVSS | FGGGTKVEIK |
| | (SEQ ID NO: 17) | (SEQ ID NO: 18) |
| ADI- | QVQLQQWGAGLLKPSETLSLTCAV | DIQMTQSPSTLSASVGDRVTITCR |
| 29401 | YGGSFSGYYWSWIRQPPGKGLEWI | ASQSIGSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYDIYPT |
| | RARGPWSFDPWGQGTLVTVSS | FGGGTKVEIK |
| | (SEQ ID NO: 19) | (SEQ ID NO: 20) |
| ADI- | QVQLQQWGAGLLKPSETLSLTCAV | DIQMTQSPSTLSASVGDRVTITCR |
| 29403 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYDSYPT |
| | RARGPWSFDPWGQGTLVTVSS | FGGGTKVEIK |
| | (SEQ ID NO: 21) | (SEQ ID NO: 22) |
| ADI- | QVQLQQWGAGLLKPSETLSLTCAV | DIQMTQSPSTLSASVGDRVTITCR |
| 29405 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYGSFPT |
| | RARGPWSFDPWGQGTLVTVSS | FGGGTKVEIK |
| | (SEQ ID NO: 23) | (SEQ ID NO: 24) |
| ADI- | QVQLQQWGAGLLKPSETLSLTCAV | DIQMTQSPSTLSASVGDRVTITCR |
| 29407 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYQSFPT |
| | RARGPWSFDPWGQGTLVTVSS | FGGGTKVEIK |
| | (SEQ ID NO: 25) | (SEQ ID NO: 26) |
| ADI- | QVQLQQWGAGLLKPSETLSLTCAV | DIQMTQSPSTLSASVGDRVTITCR |
| 29419 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYSSFST |
| | RARGPWSFDPWGQGTLVTVSS | FGGGTKVEIK |
| | (SEQ ID NO: 27) | (SEQ ID NO: 28) |
| ADI- | QVQLQQWGAGLLKPSETLSLTCAV | DIQMTQSPSTLSASVGDRVTITCR |
| 29421 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYESYST |
| | RARGPWSFDPWGQGTLVTVSS | FGGGTKVEIK |
| | (SEQ ID NO: 29) | (SEQ ID NO: 30) |
| ADI- | QVQLQQWGAGLLKPSETLSLTCAV | DIQMTQSPSTLSASVGDRVTITCR |
| 29424 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYDSFIT |
| | RARGPWSFDPWGQGTLVTVSS | FGGGTKVEIK |
| | (SEQ ID NO: 31) | (SEQ ID NO: 32) |
| ADI- | QVQLQQWGAGLLKPSETLSLTCAV | DIQMTQSPSTLSASVGDRVTITCR |
| 29425 | YGGSFSGYYWSWIRQPPGKGLEWI | ASQSISSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYQSYPT |
| | RARGPWSFDPWGQGTLVTVSS | FGGGTKVEIK |
| | (SEQ ID NO: 33) | (SEQ ID NO: 34) |
| ADI- | QVQLQQWGAGLLKPSETLSLTCAV | DIQMTQSPSTLSASVGDRVTITCR |
| 29426 | YGGSFSGYYWSWIRQPPGKGLEWI | ASQSIGSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYHSFPT |
| | RARGPWSFDPWGQGTLVTVSS | FGGGTKVEIK |
| | (SEQ ID NO: 35) | (SEQ ID NO: 36) |
| ADI- | QVQLQQWGAGLLKPSETLSLTCAV | DIQMTQSPSTLSASVGDRVTITCR |
| 29429 | YGGSFSGYYWSWIRQPPGKGLEWI | ASQSIGSWLAWYQQKPGKAPKLLI |
| | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYELYSY |
| | RARGPWSFDPWGQGTLVTVSS | TFGGGTKVEIK |
| | (SEQ ID NO: 37) | (SEQ ID NO: 38) |
| ADI- | QVQLQQWGAGLLKPSETLSLTCAV | DIQMTQSPSTLSASVGDRVTITCR |
| 29447 | YGGSFSGYYWSWIRQPPGKGLEWI | ASQSISSWLAWYQQKPGKAPKLLI |
| (F47) | GEIDHSGSTNYNPSLKSRVTISVD | YKASSLESGVPSRFSGSGSGTEFT |
| | TSKNQFSLKLSSVTAADTAVYYCA | LTISSLQPDDFATYYCQQYDTFIT |
| | RARGPWSFDPWGQGTLVTVSS | FGGGTKVEIK |
| | (SEQ ID NO: 39) | (SEQ ID NO: 40) |
| ADI- | QVQLVQSGAEVKKPGSSVKVSCKA | DIVMTQSPDSLAVSLGERATINCK |
| 27727 | SGGTFSSYAISWVRQAPGQGLEWM | SSQSVLYSSNNKNYLAWYQQKPGQ |
| | GGIIPIFGTANYAQKFQGRVTITA | PPKLLIYWASTRESGVPDRFSGSG |
| | DESTSTAYMELSSLRSEDTAVYYC | SGTDFTLTISSLQAEDVAVYYCQQ |
| | ARGDSSIRHAYYYYGMDVWGQGTT | YYSTPITFGGGTKVEIK |
| | VTVSS (SEQ ID NO: 41) | (SEQ ID NO: 42) |
| | CDR1 (SEQ ID NO: 43)- | CDR1 (SEQ ID NO: 46)- |
| | GTFSSYAIS | KSSQSVLYSSNNKNYLA |
| | CDR2 (SEQ ID NO: 44)- | CDR2 (SEQ ID NO: 47)- |
| | GIIPIFGTANYAQKFQG | WASTRES |
| | CDR3 (SEQ ID NO: 45)- | CDR3 (SEQ ID NO: 48)- |
| | ARGDSSIRHAYYYYGMDV | QQYYSTPIT |
| ADI- | QLQLQESGPGLVKPSETLSLTCTV | EIVLTQSPATLSLSPGERATLSCR |
| 29443 | SGGSISSSSYYWGWIRQPPGKGLE | ASQSVSRYLAWYQQKPGQAPRLLI |
| (F43) | WIGSIYYSGSTYYNPSLKSRVTIS | YDASNRATGIPARFSGSGSGTDFT |
| | VDTSKNQFSLKLSSVTAADTAVYY | LTISSLEPEDFAVYYCQQFDTWPP |
| | CARGSDRFHPYFDYWGQGTLVTVS | TFGGGTKVEIK |
| | S (SEQ ID NO: 49) | (SEQ ID NO: 50) |
| | CDR1 (SEQ ID NO: 51)- | CDR1 (SEQ ID NO: 54)- |
| | GSISSSSYYWG | RASQSVSRYLA |
| | CDR2 (SEQ ID NO: 52)- | CDR2 (SEQ ID NO: 55)- |
| | SIYYSGSTYYNPSLKS | DASNRAT |
| | CDR3 (SEQ ID NO: 53)- | CDR3 (SEQ ID NO: 56)- |
| | ARGSDRFHPYFDY | QQFDTWPPT |
| ADI- | QVQLQQWGAGLLKPSETLSLTCAV | DIQMTQSPSTLSASVGDRVTITCR |
| 29404 | YGGSFSGYYWSWIRQPPGKGLEWI | ASQSISSWLAWYQQKPGKAPKLLI |
| (F04) | GEIDHSGSTNYNPSLKSRVTISVD | YKASSLESGVPSRFSGSGSGTEFT |
| | TSKNQFSLKLSSVTAADTAVYYCA | LTISSLQPDDFATYYCEQYDSYPT |
| | RARGPWSFDPWGQGTLVTVSS | FGGGTKVEIK |
| | (SEQ ID NO: 57) | (SEQ ID NO: 58) |
| ADI- | QVQLVQSGAEVKKPGSSVKVSCKA | DIVMTQSPDSLAVSLGERATINCE |
| 28200 | SGGTFSSYAISWVRQAPGQGLEWM | SSQSLLNSGNQKNYLTWYQQKPGQ |
| | GGIIPIFGTANYAQKFQGRVTITA | PPKPLIYWASTRESGVPDRFSGSG |
| | DESTSTAYMELSSLRSEDTAVYYC | SGTDFTLTISSLQAEDVAVYYCQN |
| | ARRGRKASGSFYYYYGMDVWGQGT | DYSYPYTFGQGTKLEIK |
| | TVTVSS (SEQ ID NO: 59) | (SEQ ID NO: 60) |
| | CDR1 (SEQ ID NO: 517)- | CDR1 (SEQ ID NO: 520)- |
| | GTFSSYAIS | ESSQSLLNSGNQKNYLT |
| | CDR2 (SEQ ID NO: 518)- | CDR2 (SEQ ID NO: 521)- |
| | GIIPIFGTANYAQKFQG | WASTRES |
| | CDR3 (SEQ ID NO: 519)- | CDR3 (SEQ ID NO: 355)- |
| | ARRGRKASGSFYYYYGMDV | QNDYSYPYT |
| ADI- | QVQLVQSGAEVKKPGASVKVSCKA | EIVMTQSPATLSVSPGERATLSCR |
| 29379 | SGYTFTSYYMHWVRQAPGQGLEWM | ASQSVSSNLAWYQQKPGQAPRLLI |
| (E79) | GIINPSGGSTSYAQKFQGRVTMTR | YGASTRATGIPARFSGSGSGTEFT |
| | DTSTSTVYMELSSLRSEDTAVYYC | LTISSLQSEDFAVYYCQQYDDWPF |
| | ARGAPNYGDTTHDYYYMDVWGKGT | TFGGGTKVEIK |
| | TVTVSS (SEQ ID NO: 61) | (SEQ ID NO: 62) |
| | CDR1 (SEQ ID NO: 63)- | CDR1 (SEQ ID NO: 66)- |
| | YTFTSYYMH | RASQSVSSNLA |
| | CDR2 (SEQ ID NO: 64)- | CDR2 (SEQ ID NO: 67)- |
| | IINPSGGSTSYAQKFQG | GASTRAT |
| | CDR3 (SEQ ID NO: 65)- | CDR3 (SEQ ID NO: 68)- |
| | ARGAPNYGDTTHDYYYMDV | QQYDDWPFT |
| ADI- | QVQLVQSGAEVKKPGASVKVSCKA | EIVLTQSPGTLSLSPGERATLSCR |
| 29463 | SGYTFTGYYMHWVRQAPGQGLEWM | ASQSVSSNLAWYQQKPGQAPRLLI |
| (F63) | GWINPNSGGTNYAQKFQGRVTMTR | YGASTRATGIPARFSGSGSGTEFT |
| | DTSISTAYMELSRLRSDDTAVYYC | LTISSLQSEDFAVYYCQQDDYWPP |
| | ARDTGEYYDTDDHGMDVWGQGTTV | TFGGGTKVEIK |
| | TVSS (SEQ ID NO: 69) | (SEQ ID NO: 70) |
| | CDR1 (SEQ ID NO: 71)- | CDR1 (SEQ ID NO: 74)- |
| | YTFTGYYMH | RASQSVSSNLA |
| | CDR2 (SEQ ID NO: 72)- | CDR2 (SEQ ID NO: 75)- |
| | WINPNSGGTNYAQKFQG | GASTRAT |
| | CDR3 (SEQ ID NO: 73)- | CDR3 (SEQ ID NO: 76)- |
| | ARDTGEYYDTDDHGMDV | QQDDYWPPT |
| ADI- | EVQLLESGGGLVQPGGSLRLSCAA | DIQMTQSPSSVSASVGDRVTITCR |
| 27744 | SGFTFSSYAMSWVRQAPGKGLEWV | ASQGIDSWLAWYQQKPGKAPKLLI |
| (A44) | SAISGSGGSTYYADSVKGRFTISR | YAASSLQSGVPSRFSGSGSGTDFT |
| | DNSKNTLYLQMNSLRAEDTAVYYC | LTISSLQPEDFATYYCQQGVSYPR |
| | AKDGGYYDSGAGDYWGQGTLVTVS | TFGGGTKVEIK |
| | S (SEQ ID NO: 77) | (SEQ ID NO: 78) |
| | CDR1 (SEQ ID NO: 79)- | CDR1 (SEQ ID NO: 82)- |
| | FTFSSYAMS | RASQGIDSWLA |
| | CDR2 (SEQ ID NO: 80)- | CDR2 (SEQ ID NO: 83)- |
| | AISGSGGSTYYADSVKG | AASSLQS |
| | CDR3 (SEQ ID NO: 81)- | CDR3 (SEQ ID NO: 84)- |
| | AKDGGYYDSGAGDY | QQGVSYPRT |
| ADI- | EVQLVESGGGLVKPGGSLRLSCAA | DIQMTQSPSSVSASVGDRVTITCR |
| 27749 | SGFTFSSYSMNWVRQAPGKGLEWV | ASQGISSWLAWYQQKPGKAPKLLI |
| (A49) | SSISSSSSYIYYADSVKGRFTISR | YAASSLQSGVPSRFSGSGSGTDFT |
| | DNAKNSLYLQMNSLRAEDTAVYYC | LTISSLQPEDFATYYCQQGVSFPR |
| | ARGAPMGAAAGWFDPWGQGTLVTV | TFGGGTKVEIK |
| | SS (SEQ ID NO: 85) | (SEQ ID NO: 86) |
| | CDR1 (SEQ ID NO: 87)- | CDR1 (SEQ ID NO: 90)- |
| | FTFSSYSMN | RASQGISSWLA |
| | CDR2 (SEQ ID NO: 88)- | CDR2 (SEQ ID NO: 91)- |
| | SISSSSSYIYYADSVKG | AASSLQS |
| | CDR3 (SEQ ID NO: 89)- | CDR3 (SEQ ID NO: 92)- |
| | ARGAPMGAAAGWFDP | QQGVSFPRT |
| ADI- | QVQLVQSGAEVKKPGASVKVSCKA | EIVLTQSPATLSLSPGERATLSCR |
| 29378 | SGYTFTSYYMHWVRQAPGQGLEWM | ASQSVSSYLAWYQQKPGQAPRLLI |
| (E78) | GIINPSGGSTSYAQKFQGRVTMTR | YDASNRATGIPARFSGSGSGTDFT |
| | DTSTSTVYMELSSLRSEDTAVYYC | LTISSLEPEDFAVYYCQQSDNWPF |
| | AREGAGFAYGMDYYYMDVWGKGTT | TFGGGTKVEIK |
| | VTVSS (SEQ ID NO: 93) | (SEQ ID NO: 94) |
| | CDR1 (SEQ ID NO: 95)- | CDR1 (SEQ ID NO: 98)- |
| | YTFTSYYMH | RASQSVSSYLA |
| | CDR2 (SEQ ID NO: 96)- | CDR2 (SEQ ID NO: 99)- |
| | IINPSGGSTSYAQKFQG | DASNRAT |
| | CDR3 (SEQ ID NO: 97)- | CDR3 (SEQ ID NO: 100)- |
| | AREGAGFAYGMDYYYMDV | QQSDNWPFT |

Alternatively, a heavy chain variable domain represented by SEQ ID NO:101 can be paired with a light chain variable domain represented by SEQ ID NO:102 to form an antigen-binding site that can bind to NKG2D, as illustrated in U.S. Pat. No. 9,273,136.

SEQ ID NO: 101
QVQLVESGGGLVKPGGSLRLSCAASGFTFSSYGMHWVRQAPGKGLEWVAF
IRYDGSNKYYADSVKGRFTISRDNSKNTLYLQMNSLRAEDTAVYYCAKDR
GLGDGTYFDYWGQGTTVTVSS
SEQ ID NO: 102
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL

Alternatively, a heavy chain variable domain represented by SEQ ID NO:103 can be paired with a light chain variable domain represented by SEQ ID NO:104 to form an antigen-binding site that can bind to NKG2D, as illustrated in U.S. Pat. No. 7,879,985.

SEQ ID NO: 103
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS
SEQ ID NO: 104
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK

Table 2 lists peptide sequences of heavy chain variable domains and light chain variable domains that, in combination, can bind to CXCR4.

TABLE 2

| | Heavy chain | Light chain |
|---|---|---|
| | variable domain | variable domain |
| Clones | amino acid sequence | amino acid sequence |
| Ulocuplumab | EVQLVESGGGLVQPGGSLRLSCAA | DIQMTQSPSSLSASVGDRVTITCR |
| | AGFTFSSYSMNWVRQAPGKGLEWV | ASQGISSWLAWYQQKPEKAPKSLI |
| | SYISSRSRTIYYADSVKGRFTISR | YAASSLQSGVPSRFSGSGSGTDFT |
| | DNAKNSLYLQMNSLRDEDTAVYYC | LTISSLQPEDFVTYYCQQYNSYPR |
| | ARDYGGQPPYYYYYGMDVWGQGTT | TFGQGTKVEIKR |
| | VTVSSA (SEQ ID NO: 109) | (SEQ ID NO: 110) |
| | CDR1 (SEQ ID NO: 111)- | CDR1 (SEQ ID NO: 114)- |
| | GFTFSSY | QGISSWLA |
| | CDR2 (SEQ ID NO: 112)- | CDR2 (SEQ ID NO: 115)- |
| | SSRSRT | AASSLQS |
| | CDR3 (SEQ ID NO: 113)- | CDR3 (SEQ ID NO: 116)- |
| | DYGGQPPYYYYYGMDV | QQYNSYPRT |
| anti-CXCR4 | QVQLVQSGAEVKKPGASVKVSCKA | SSELTQDPAVSVALGQTVRITCQG |
| (U.S. Pat. No. | SGYTFTSYGISWVRQAPGQGLEWM | DSLRKFFASWYQQKPGQAPVLVIY |
| 8,329,178) | GWISAYNGNTNYAQKLQGRVTMTT | GKNSRPSGIPDRFSGSNSRNTASL |
| | DTSTSTAYMELRSLRSDDTAVYYC | TITGAQAEDEGDYYCNSRDSRDNH |
| | ARDTPGIAARRYYYYGMDVWGQGT | QVFGAGTKVTVLS |
| | TVTVSS (SEQ ID NO: 117) | (SEQ ID NO: 118) |
| | CDR1 (SEQ ID NO: 119)- | CDR1 (SEQ ID NO: 122)- |
| | GFTFSSY | SLRKFFAS |
| | CDR2 (SEQ ID NO: 120)- | CDR2 (SEQ ID NO: 123)- |
| | SAYNGN | GKNSRPS |
| | CDR3 (SEQ ID NO: 121)- | CDR3 (SEQ ID NO: 124)- |
| | DTPGIAARRYYYYGMDV | NSRDSRDNHQV |
| anti-CXCR4 | EVQLVESGGGLVQPGGSLRLSCAA | DIVMTQSPDSLAVSLGERATINCK |
| (WO2009140124) | SGFTSTDYYFSWVRQAPGKGLEWV | SSQSLFNSRTRKKYLAWYQQKPGQ |
| | GFIRTKSKGYTTEYSGSVKGRFTI | PPKLLIYWASKRKSGVPDRFSGSG |
| | SRDDSKNSLYLQMNSLKTEDTAVY | SGTDFTLTISSLQAEDVAVYYCKQ |
| | YCAREPITTDPRDYWGQGTLVTVS | SRFLRAFGQGTKLEIK |
| | S (SEQ ID NO: 125) | (SEQ ID NO: 126) |
| | CDR1 (SEQ ID NO: 127)- | CDR1 (SEQ ID NO: 130)- |
| | GFTSTDYYFS | KSSQSLFNSRTRKKYL |
| | CDR2 (SEQ ID NO: 128)- | CDR2 (SEQ ID NO: 131)- |
| | FIRTKSKGYTTEYSGSVKG | WASKRKS |
| | CDR3 (SEQ ID NO: 129)- | CDR3 (SEQ ID NO: 132)- |
| | EPITTDPRDY | KQSRFLRA |
| US 2011/0020218A1 | EVQLVESGGGLVQPGRSLRLSCTA | DIVMTQSPSSLAVSLGERATMSCK |
| | SGFTFTDNYMSWVRQAPGKGLEWV | SSQSLFNSRTRKNYLAWYQQKPGQ |
| | GFIRNKANGYTTEYAASVKGRFTI | SPKLLIYWASARDSGVPARFTGSG |
| | SRDNSKSIAYLQMNSLKTEDTAVY | SETYFTLTISRVQAEDLAVYYCMQ |
| | YCARDVGSNYFDYWGQGTLVTVSS | SFNLRTFGQGTKVEIK |
| | (SEQ ID NO: 522) | (SEQ ID NO: 526) |
| | CDR1 (SEQ ID NO: 523): | CDR1 (SEQ ID NO: 527): |
| | FTFTDNYMS | KSSQSLFNSRTRKNYLA |
| | CDR2 (SEQ ID NO: 524): | CDR2 (SEQ ID NO: 528): |
| | FIRNKANGYTTEYAASV | WASARDS |
| | CDR3 (SEQ ID NO: 525): | CRD3 (SEQ ID NO: 529): |
| | ARDVGSNYFDY | MQSFNLRT |

Alternatively, novel antigen-binding sites that can bind to CXCR4 can be identified by screening for binding to the amino acid sequence defined by SEQ ID NO:133.

SEQ ID NO: 133
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGKRGGHSSVSTESESSSFH
SS

Table 3 lists peptide sequences of heavy chain variable domains and light chain variable domains that, in combination, can bind to CD25.

TABLE 3

| | Heavy chain | Light chain |
|---|---|---|
| | variable domain | variable domain |
| Clones | amino acid sequence | amino acid sequence |
| Daclizumab | QVQLVQSGAEVKKPGSSVKVSCKA | DIQMTQSPSTLSASVGDRVTITCS |
| | SGYTFTSYRMHWVRQAPGQGLEWI | ASSSISYMHWYQQKPGKAPKLLIY |
| | GYINPSTGYTEYNQKFKDKATITA | TTSNLASGVPARFSGSGSGTEFTL |
| | DESTNTAYMELSSLRSEDTAVYYC | TISSLQPDDFATYYCHQRSTYPLT |
| | ARGGGVFDYWGQGTLVTVSSA | FGQGTKVEVKR |
| | (SEQ ID NO: 134) | (SEQ ID NO: 135) |
| | CDR1 (SEQ ID NO: 136)- | CDR1 (SEQ ID NO: 139)- |
| | GYTFTSY | SSISYMH |
| | CDR2 (SEQ ID NO: 137)- | CDR2 (SEQ ID NO: 140)- |
| | NPSTGY | TTSNLAS |
| | CDR3 (SEQ ID NO: 138)- | CDR3 (SEQ ID NO: 141)- |
| | GGGVFDY | HQRSTYPLT |
| Basiliximab | QLQQSGTVLARPGASVKMSCKASG | QIVSTQSPAIMSASPGEKVTMTCS |
| | YSFTRYWMHWIKQRPGQGLEWIGA | ASSSRSYMQWYQQKPGTSPKRWIY |
| | IYPGNSDTSYNQKFEGKAKLTAVT | DTSKLASGVPARFSGSGSGTSYSL |
| | SASTAYMELSSLTHEDSAVYYCSR | TISSMEAEDAATYYCHQRSSYTFG |
| | DYGYYFDFWGQGTTLTVSSA | GGTKLEIKR |
| | (SEQ ID NO: 142) | (SEQ ID NO: 143) |
| | CDR1 (SEQ ID NO: 144)- | CDR1 (SEQ ID NO: 147)- |
| | GYSFTRY | SSRSYMQ |
| | CDR2 (SEQ ID NO: 145)- | CDR2 (SEQ ID NO: 148)- |
| | YPGNSD | DTSKLAS |
| | CDR3 (SEQ ID NO: 146)- | CDR3 (SEQ ID NO: 149)- |
| | DYGYYFDF | HQRSSYT |
| Camidanlumab | QVQLVQSGAEVKKPGSSVKVSCKA | EIVLTQSPGTLSLSPGERATLSCR |
| | SGGTFSRYIINWVRQAPGQGLEWM | ASQSVSSYLAWYQQKPGQAPRLLI |
| | GRIIPILGVENYAQKFQGRVTITA | YGASSRATGIPDRFSGSGSGTDFT |
| | DKSTSTAYMELSSLRSEDTAVYYC | LTISRLEPEDFAVYYCQQYGSSPL |
| | ARKDWFDYWGQGTLVTVSSA | TFGGGTKVEIKR |
| | (SEQ ID NO: 150) | (SEQ ID NO: 151) |
| | CDR1 (SEQ ID NO: 152)- | CDR1 (SEQ ID NO: 155)- |
| | GGTFSRYIIN | CRASQSVSSYLA |
| | CDR2 (SEQ ID NO: 153)- | CDR2 (SEQ ID NO: 156)- |
| | RIIPILGVENYAQKFQG | GASSRAT |
| | CDR3 (SEQ ID NO: 154)- | CDR3 (SEQ ID NO: 157)- |
| | KDWFDY | QQYGSSPLT |

Alternatively, novel antigen-binding sites that can bind to CD25 can be identified by screening for binding to the amino acid sequence defined by SEQ ID NO:158.

SEQ ID NO: 158
MDSYLLMWGLLTFIMVPGCQAELCDDDPPEIPHATFKAMAYKEGTMLNCE
CKRGFRRIKSGSLYMLCTGNSSHSSWDNQCQCTSSATRNTTKQVTPQPEE
QKERKTTEMQSPMQPVDQASLPGHCREPPPWENEATERIYHFVVGQMVYY
QCVQGYRALHRGPAESVCKMTHGKTRWTQPQLICTGEMETSQFPGEEKPQ
ASPEGRPESETSCLVTTTDFQIQTEMAATMETSIFTTEYQVAVAGCVFLL
ISVLLLSGLTWQRRQRKSRRTI

Antigen-binding sites that can bind to tumor associated antigen VLA4 can be identified by screening for binding to the amino acid sequence defined by SEQ ID NO:159 or SEQ ID NO:160.

SEQ ID NO: 159
MAWEARREPGPRRAAVRETVMLLLCLGVPTGRPYNVDTESALLYQGPHNT
LFGYSVVLHSHGANRWLLVGAPTANWLANASVINPGAIYRCRIGKNPGQT
CEQLQLGSPNGEPCGKTCLEERDNQWLGVTLSRQPGENGSIVTCGHRWKN
IFYIKNENKLPTGGCYGVPPDLRTELSKRIAPCYQDYVKKFGENFASCQA
GISSFYTKDLIVMGAPGSSYWTGSLFVYNITTNKYKAFLDKQNQVKFGSY
LGYSVGAGHFRSQHTTEVVGGAPQHEQIGKAYIFSIDEKELNILHEMKGK
KLGSYFGASVCAVDLNADGFSDLLVGAPMQSTIREEGRVFVYINSGSGAV
MNAMETNLVGSDKYAARFGESIVNLGDIDNDGFEDVAIGAPQEDDLQGAI
YIYNGRADGISSTFSQRIEGLQISKSLSMFGQSISGQIDADNNGYVDVAV
GAFRSDSAVLLRTRPVVIVDASLSHPESVNRTKFDCVENGWPSVCIDLTL
CFSYKGKEVPGYIVLFYNMSLDVNRKAESPPRFYFSSNGTSDVITGSIQV
SSREANCRTHQAFMRKDVRDILTPIQIEAAYHLGPHVISKRSTEEFPPLQ
PILQQKKEKDIMKKTINFARFCAHENCSADLQVSAKIGFLKPHENKTYLA
VGSMKTLMLNVSLFNAGDDAYETTLHVKLPVGLYFIKILELEEKQINCEV
TDNSGVVQLDCSIGYIYVDHLSRIDISFLLDVSSLSRAEEDLSITVHATC
ENEEEMDNLKHSRVTVAIPLKYEVKLTVHGFVNPTSFVYGSNDENEPETC
MVEKMNLTFHVINTGNSMAPNVSVEIMVPNSFSPQTDKLFNILDVQTTTG
ECHFENYQRVCALEQQKSAMQTLKGIVRFLSKTDKRLLYCIKADPHCLNF
LCNFGKMESGKEASVHIQLEGRPSILEMDETSALKFEIRATGFPEPNPRV
IELNKDENVAHVLLEGLHHQRPKRYFTIVIISSSLLLGLIVLLLISYVMW
KAGFFKRQYKSILQEENRRDSWSYINSKSNDD
SEQ ID NO: 160
MNLQPIFWIGLISSVCCVFAQTDENRCLKANAKSCGECIQAGPNCGWCTN
STFLQEGMPTSARCDDLEALKKKGCPPDDIENPRGSKDIKKNKNVTNRSK
GTAEKLKPEDITQIQPQQLVLRLRSGEPQTFTLKFKRAEDYPIDLYYLMD
LSYSMKDDLENVKSLGTDLMNEMRRITSDFRIGFGSFVEKTVMPYISTTP
AKLRNPCTSEQNCTSPFSYKNVLSLTNKGEVFNELVGKQRISGNLDSPEG
GFDAIMQVAVCGSLIGWRNVTRLLVFSTDAGFHFAGDGKLGGIVLPNDGQ
CHLENNMYTMSHYYDYPSIAHLVQKLSENNIQTIFAVTEEFQPVYKELKN
LIPKSAVGTLSANSSNVIQLIIDAYNSLSSEVILENGKLSEGVTISYKSY
CKNGVNGTGENGRKCSNISIGDEVQFEISITSNKCPKKDSDSFKIRPLGF
TEEVEVILQYICECECQSEGIPESPKCHEGNGTFECGACRCNEGRVGRHC
ECSTDEVNSEDMDAYCRKENSSEICSNNGECVCGQCVCRKRDNTNEIYSG
KFCECDNFNCDRSNGLICGGNGVCKCRVCECNPNYTGSACDCSLDTSTCE
ASNGQICNGRGICECGVCKCTDPKFQGQTCEMCQTCLGVCAEHKECVQCR
AFNKGEKKDTCTQECSYFNITKVESRDKLPQPVQPDPVSHCKEKDVDDCW
FYFTYSVNGNNEVMVHVVENPECPTGPDIIPIVAGVVAGIVLIGLALLLI
WKLLMIIHDRREFAKFEKEKMNAKWDTGENPIYKSAVTTVVNPKYEGK

Antigen-binding sites that can bind to tumor associated antigen CD44 can be identified by screening for binding to the amino acid sequence defined by SEQ ID NO:161.

SEQ ID NO: 161
MDKFWWHAAWGLCLVPLSLAQIDLNITCRFAGVFHVEKNGRYSISRTEAA
DLCKAFNSTLPTMAQMEKALSIGFETCRYGFIEGHVVIPRIHPNSICAAN
NTGVYILTSNTSQYDTYCFNASAPPEEDCTSVTDLPNAFDGPITITIVNR
DGTRYVQKGEYRTNPEDIYPSNPTDDDVSSGSSSERSSTSGGYIFYTFST
VHPIPDEDSPWITDSTDRIPATTLMSTSATATETATKRQETWDWFSWLFL
PSESKNHLHTTTQMAGTSSNTISAGWEPNEENEDERDRHLSFSGSGIDDD
EDFISSTISTTPRAFDHTKQNQDWTQWNPSHSNPEVLLQTTTRMTDVDRN
GTTAYEGNWNPEAHPPLIHHEHHEEEETPHSTSTIQATPSSTTEETATQK
EQWFGNRWHEGYRQTPKEDSHSTTGTAAASAHTSHPMQGRTTPSPEDSSW
TDFFNPISHPMGRGHQAGRRMDMDSSHSITLQPTANPNTGLVEDLDRTGP
LSMTTQQSNSQSFSTSHEGLEEDKDHPTTSTLTSSNRNDVTGGRRDPNHS
EGSTTLLEGYTSHYPHTKESRTFIPVTSAKTGSFGVTAVTVGDSNSNVNR
SLSGDQDTFHPSGGSHTTHGSESDGHSHGSQEGGANTTSGPIRTPQIPEW
LIILASLLALALILAVCIAVNSRRRCGQKKKLVINSGNGAVEDRKPSGLN
GEASKSQEMVHLVNKESSETPDQFMTADETRNLQNVDMKIGV

Antigen-binding sites that can bind to tumor associated antigen CD13 can be identified by screening for binding to the amino acid sequence defined by SEQ ID NO:162.

SEQ ID NO: 162
MAKGFYISKSLGILGILLGVAAVCTIIALSVVYSQEKNKNANSSPVASTT
PSASATTNPASATTLDQSKAWNRYRLPNTLKPDSYRVTLRPYLTPNDRGL
YVFKGSSTVRFTCKEATDVIIIHSKKLNYTLSQGHRVVLRGVGGSQPPDI
DKTELVEPTEYLVVHLKGSLVKDSQYEMDSEFEGELADDLAGFYRSEYME
GNVRKVVATTQMQAADARKSFPCFDEPAMKAEFNITLIHPKDLTALSNML
PKGPSTPLPEDPNWNVTEFHTTPKMSTYLLAFIVSEFDYVEKQASNGVLI
RIWARPSAIAAGHGDYALNVTGPILNFFAGHYDTPYPLPKSDQIGLPDFN
AGAMENWGLVTYRENSLLFDPLSSSSSNKERVVTVIAHELAHQWFGNLVT
IEWWNDLWLNEGFASYVEYLGADYAEPTWNLKDLMVLNDVYRVMAVDALA
SSHPLSTPASEINTPAQISELFDAISYSKGASVLRMLSSFLSEDVFKQGL
ASYLHTFAYQNTIYLNLWDHLQEAVNNRSIQLPTTVRDIMNRWTLQMGFP
VITVDTSTGTLSQEHFLLDPDSNVTRPSEFNYVWIVPITSIRDGRQQQDY
WLIDVRAQNDLFSTSGNEWVLLNLNVTGYYRVNYDEENWRKIQTQLQRDH
SAIPVINRAQIINDAFNLASAHKVPVTLALNNTLFLIEERQYMPWEAALS
SLSYFKLMFDRSEVYGPMKNYLKKQVTPLFIHFRNNTNNWREIPENLMDQ
YSEVNAISTACSNGVPECEEMVSGLFKQWMENPNNNPIHPNLRSTVYCNA
IAQGGEEEWDFAWEQFRNATLVNEADKLRAALACSKELWILNRYLSYTLN
PDLIRKQDATSTIISITNNVIGQGLVWDFVQSNWKKLFNDYGGGSFSFSN
LIQAVTRRFSTEYELQQLEQFKKDNEETGFGSGTRALEQALEKTKANIKW
VKENKEVVLQWFTENSK

Antigen-binding sites that can bind to tumor associated antigen CD15 can be identified by screening for binding to 3-fucosyl-N-acetyl-lactosamine.

Antigen-binding sites that can bind to tumor associated antigen CD47 can be identified by screening for binding to the amino acid sequence defined by SEQ ID NO:163.

SEQ ID NO: 163
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

Antigen-binding sites that can bind to tumor associated antigen CD81 can be identified by screening for binding to the amino acid sequence defined by SEQ ID NO:165.

SEQ ID NO: 165
MGVEGCTKCIKYLLFVFNFVFWLAGGVILGVALWLRHDPQTTNLLYLELG
DKPAPNTFYVGIYILIAVGAVMMFVGFLGCYGAIQESQCLLGTFFTCLVI
LFACEVAAGIWGFVNKDQIAKDVKQFYDQALQQAVVDDDANNAKAVVKTF
HETLDCCGSSTLTALTTSVLKNNLCPSGSNIISNLFKEDCHQKIDDLFSG
KLYLIGIAAIVVAVIMIFEMILSMVLCCGIRNSSVY

Alternatively, Table 4 lists peptide sequences of heavy chain variable domains and light chain variable domains that, in combination, can bind to VLA4 (Natalizumab), CD44 (Bivatuzumab), or CD47.

TABLE 4

| | Heavy chain | Light chain |
|---|---|---|
| | variable domain | variable domain |
| | amino acid | amino acid |
| Clones | sequence | sequence |
| Natalizumab | VKLQQSGAELVKPGASV | SIVMTQTPKFLLVSAGD |
| | KLFCTASGFNIKDTYMH | RVTITCKASQSVTNDVA |
| | WVKQRPQQGLEWIGRID | WYQQKPGQSPKLLIYYA |
| | PASGDTKYDPKFQVKAT | SNRYTGVPDRFTGSGYG |
| | ITADTSSNTAWLQLSSL | TDFTFTISTVQAEDLAV |
| | TSEDTAVYYCADGMWVS | YFCQQDYS |
| | TGYALDFWGQGTTVTVS | SPYTFGGGTKLEI |
| | S | (SEQ ID NO: 167) |
| | (SEQ ID NO: 166) | CDR1 |
| | CDR1 | (SEQ ID NO: 171)- |
| | (SEQ ID NO: 168)- | QSVTNDVA |
| | GFNIKDT | CDR2 |
| | CDR2 | (SEQ ID NO: 172)- |
| | (SEQ ID NO: 169)- | YASNRYT |
| | DPASGD | CDR3 |
| | CDR3 | (SEQ ID NO: 173)- |
| | (SEQ ID NO: 170)- | |
| | GMWVSTGYALDF | |
| Bivatuzumab | EVQLVESGGGLVKPGGS | EIVLTQSPATLSLSPGE |
| | LRLSCAASGFTFSSYDM | RATLSCSASSSINYIYW |
| | SWVRQAPGKGLEWVSTI | YQQKPGQAPRLLIYLTS |
| | SSGGSYTYYLDSIKGRF | NLASGVPARFSGSGSGT |
| | TISRDNAKNSLYLQMNS | DFTLTISSLEPEDFAVY |
| | LRAEDTAVYYCARQGLD | YCLQWSSNPLTFGGGTK |
| | YWGRGTLVTVSSA | VEIKR |
| | (SEQ ID NO: 174) | (SEQ ID NO: 175) |
| | CDR1 | CDR1 |
| | (SEQ ID NO: 176)- | (SEQ ID NO: 179)- |
| | GFTFSSY | SSINYIY |
| | CDR2 | CDR2 |
| | (SEQ ID NO: 177)- | (SEQ ID NO: 180)- |
| | SSGGSY | LTSNLAS |
| | CDR3 | CDR3 |
| | (SEQ ID NO: 178)- | (SEQ ID NO: 181)- |
| | QGLDY | LQWSSNPLT |
| Anti-CD47 | QVQLVQSGAEVKKPGAS | DIVMTQSPLSLPVTPGE |
| (WO | VKVSCKASGYTFTNYNM | PASISCRSSQSIVYSNG |
| 2011143624) | HWVRQAPGQRLEWMGTI | NTYLGWYLQKPGQSPQL |
| | YPGNDDTSYNQKFKDRV | LIYKVSNRFSGVPDRFS |
| | TITADTSASTAYMELSS | GSGSTDFTLKISRVEGA |
| | LRSEDTAVYYCARGGYR | EDVGVYYCFQGSHVPYT |
| | AMDYWGQGTLVTVSS | FGQGTKLEIK |
| | (SEQ ID NO: 182) | (SEQ ID NO: 183) |
| | CDR1 | CDR1 |
| | (SEQ ID NO: 184)- | (SEQ ID NO: 187)- |
| | GYTFTNYNMH | RSSQSIVYSNGNTYLG |
| | CDR2 | CDR2 |
| | (SEQ ID NO: 185)- | (SEQ ID NO: 188)- |
| | TIYPGNDDTSYNQKFKD | KVSNRFS |
| | CDR3 | CDR3 |
| | (SEQ ID NO: 186)- | (SEQ ID NO: 189)- |
| | GGYRAMDY | FQGSHVPYT |

Antigen-binding sites that can bind to tumor associated antigen CD23 can be identified by screening for binding to the amino acid sequence defined by SEQ ID NO:190.

SEQ ID NO: 190
MEEGQYSEIEELPRRRCCRRGTQIVLLGLVTAALWAGLLTLLLLWHWDTT
QSLKQLEERAARNVSQVSKNLESHHGDQMAQKSQSTQISQELEELRAEQQ
RLKSQDLELSWNLNGLQADLSSFKSQELNERNEASDLLERLREEVTKLRM
ELQVSSGFVCNTCPEKWINFQRKCYYFGKGTKQWVHARYACDDMEGQLVS
IHSPEEQDFLTKHASHTGSWIGLRNLDLKGEFIWVDGSHVDYSNWAPGEP
TSRSQGEDCVMMRGSGRWNDAFCDRKLGAWVCDRLATCTPPASEGSAESM
GPDSRPDPDGRLPTPSAPLHS

Antigen-binding sites that can bind to tumor associated antigen CD40 can be identified by screening for binding to the amino acid sequence defined by SEQ ID NO:191.

SEQ ID NO: 191
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ

Antigen-binding sites that can bind to tumor associated antigen CD70 can be identified by screening for binding to the amino acid sequence defined by SEQ ID NO:192.

SEQ ID NO: 192
MPEEGSGCSVRRRPYGCVLRAALVPLVAGLVICLVVCIQRFAQAQQQLPL
ESLGWDVAELQLNHTGPQQDPRLYWQGGPALGRSFLHGPELDKGQLRIHR
DGIYMVHIQVTLAICSSTTASRHHPTTLAVGICSPASRSISLLRLSFHQG
CTIASQRLTPLARGDTLCTNLTGTLLPSRNTDETFFGVQWVRP

Antigen-binding sites that can bind to tumor associated antigen CD79a can be identified by screening for binding to the amino acid sequence defined by SEQ ID NO:193.

SEQ ID NO: 193
MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGTLLLFRKRWQNEKLGLDAGDEYEDENLYEGLNLDDCSMYE
DISRGLQGTYQDVGSLNIGDVQLEKP

Antigen-binding sites that can bind to tumor associated antigen CD79b can be identified by screening for binding to the amino acid sequence defined by SEQ ID NO:194.

SEQ ID NO: 194
MARLALSPVPSHWMVALLLLLSAEPVPAARSEDRYRNPKGSACSRIWQSP
RFIARKRGFTVKMHCYMNSASGNVSWLWKQEMDENPQQLKLEKGRMEESQ
NESLATLTIQGIRFEDNGIYFCQQKCNNTSEVYQGCGTELRVMGFSTLAQ
LKQRNTLKDGIIMIQTLLIILFIIVPIFLLLDKDDSKAGMEEDHTYEGLD
IDQTATYEDIVTLRTGEVKWSVGEHPGQE

Antigen-binding sites that can bind to tumor associated antigen CD80 can be identified by screening for binding to the amino acid sequence defined by SEQ ID NO:195.

SEQ ID NO: 195
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

Antigen-binding sites that can bind to tumor associated antigen CRLF2 can be identified by screening for binding to the amino acid sequence defined by SEQ ID NO:196.

SEQ ID NO: 196
MGRLVLLWGAAVFLLGGWMALGQGGAAEGVQIQIIYFNLETVQVTWNASK
YSRTNLTFHYRFNGDEAYDQCTNYLLQEGHTSGCLLDAEQRDDILYFSIR
NGTHPVFTASRWMVYYLKPSSPKHVRFSWHQDAVTVTCSDLSYGDLLYEV
QYRSPFDTEWQSKQENTCNVTIEGLDAEKCYSFWVRVKAMEDVYGPDTYP
SDWSEVTCWQRGEIRDACAETPTPPKPKLSKFILISSLAILLMVSLLLLS
LWKLWRVKKFLIPSVPDPKSIFPGLFEIHQGNFQEWITDTQNVAHLHKMA
GAEQESGPEEPLVVQLAKTEAESPRMLDPQTEEKEASGGSLQLPHQPLQG
GDVVTIGGFTFVMNDRSYVAL

Alternatively, table 5 lists peptide sequences of heavy chain variable domains and light chain variable domains that, in combination, can bind to CD23 (lumiliximab), CD40 (dacetuzumab, selicrelumab, lucatumumab, bleselumab), CD70 (vorsetuzumab), CD79b (polatuzumab), CD80 (galiximab), or CRLF2 (US20160046720).

TABLE 5

| | Heavy chain | Light chain |
|---|---|---|
| | variable domain | variable domain |
| | amino acid | amino acid |
| Clones | sequence | sequence |
| lumiliximab | EVQLVESGGGLAKPGGS | DIQMTQSPSSLSASVGD |
| | LRLSCAASGFRFTFNNY | RVTITCRASQDIRYYLN |
| | YMDWVRQAPGQGLEWVS | WYQQKPGKAPKLLIYVA |
| | RISSSGDPTWYADSVKG | SSLQSGVPSRFSGSGSG |
| | RFTISRENANNTLFLQM | TEFTLTVSSLQPEDFAT |
| | NSLRAEDTAVYYCASLT | YYCLQVYSTPRTFGQGT |
| | TGSDSWGQGVLVTVSS | KVEIK |
| | (SEQ ID NO: 197) | (SEQ ID NO: 198) |
| | CDR1 | CDR1 |
| | (SEQ ID NO: 199)- | (SEQ ID NO: 202)- |
| | GFRFTFNNY | QDIRYYLN |
| | CDR2 | CDR2 |
| | (SEQ ID NO: 200)- | (SEQ ID NO: 203)- |
| | SSSGDP | VASSLQS |
| | CDR3 | CDR3 |
| | (SEQ ID NO: 201)- | (SEQ ID NO: 204)- |
| | LTTGSDS | LQVYSTPRT |
| dacetuzumab | EVQLVESGGGLVQPGGS | DIQMTQSPSSLSASVGD |
| | LRLSCAASGYSFTGYYI | RVTITCRSSQSLVHSNG |
| | HWVRQAPGKGLEWVARV | NTFLHWYQQKPGKAPKL |
| | IPNAGGTSYNQKFKGRF | LIYTVSNRFSGVPSRFS |
| | TLSVDNSKNTAYLQMNS | GSGSGTDFTLTISSLQP |
| | LRAEDTAVYYCAREGIY | EDFATYFCSQTTHVPWT |
| | WWGQGTLVTVSSA | FGQGTKVEIKR |
| | (SEQ ID NO: 205) | (SEQ ID NO: 206) |
| | CDR1 | CDR1 |
| | (SEQ ID NO: 207)- | (SEQ ID NO: 210)- |
| | GYSFTGY | QSLVHSNGNTFLH |
| | CDR2 | CDR2 |
| | (SEQ ID NO: 208)- | (SEQ ID NO: 211)- |
| | IPNAGG | TVSNRFS |
| | CDR3 | CDR3 |
| | (SEQ ID NO: 209)- | (SEQ ID NO: 212)- |
| | EGIYW | SQTTHVPWT |
| selicrelumab | QVQLVQSGAEVKKPGAS | DIQMTQSPSSVSASVGD |
| | VKVSCKASGYTFTGYYM | RVTITCRASQGIYSWLA |
| | HWVRQAPGQGLEWMGWI | WYQQKPGKAPNLLIYTA |
| | NPDSGGTNYAQKFQGRV | STLQSGVPSRFSGSGSG |
| | TMTRDTSISTAYMELNR | TDFTLTISSLQPEDFAT |
| | LRSDDTAVYYCARDQPL | YYCQQANIFPLTFGGGT |
| | GYCTNGVCSYFDYWGQG | KVEIKR |
| | TLVTVSSA | (SEQ ID NO: 214) |
| | (SEQ ID NO: 213) | CDR1 |
| | CDR1 | (SEQ ID NO: 218)- |
| | (SEQ ID NO: 215)- | QGIYSWLA |
| | GYTFTGY | CDR2 |
| | CDR2 | (SEQ ID NO: 219)- |
| | (SEQ ID NO: 216)- | TASTLQS |
| | NPDSGG | CDR3 |
| | CDR3 | (SEQ ID NO: 220)- |
| | (SEQ ID NO: 217)- | QQANIFPLT |
| | DQPLGYCTNGVCSYFDY | |
| lucatumumab | QVQLVESGGGVVQPGRS | DIVMTQSPLSLTVTPGE |
| | LRLSCAASGFTFSSYGM | PASISCRSSQSLLYSNG |
| | HWVRQAPGKGLEWVAVI | YYNYLDWLQKPGQSPQV |
| | SYEESNRYHADSVKGRF | LISLGSNRASGVPDRFS |
| | TISRDNSKITLYLQMNS | GSGSGTDFTLKISRVEA |
| | LRTEDTAVYYCARDGGI | EDVGVYYCMQARQTPFT |
| | AAPGPDYWGQGTLVTVS | FGPGTKVDIRR |
| | SA | (SEQ ID NO: 222) |
| | (SEQ ID NO: 221) | CDR1 |
| | CDR1 | (SEQ ID NO: 226)- |
| | (SEQ ID NO: 223)- | QSLLYSNGYNYLD |
| | GFTFSSY | CDR2 |
| | CDR2 | (SEQ ID NO: 227)- |
| | (SEQ ID NO: 224)- | LGSNRAS |
| | SYEESN | CDR3 |
| | CDR3 | (SEQ ID NO: 228)- |
| | (SEQ ID NO: 225)- | MQARQTPFT |
| | DGGIAAPGPDY | |
| Bleselumab | QVQLQQSGPGLVKPSQT | EIVLTQSPATLSLSPGE |
| ASKP1240 | LSLTCAISGDSVSSNSA | RATLSCRASQSVSSYLA |
| | TWNWIRQSPSRDLEWLG | WYQQKPGQAPRLLIYDA |
| | RTYYRSKWYRDYVGSVK | SNRATGIPARFSGSGSG |
| | SRIIINPDTSNNQFSLQ | TDFTLTISSLEPEDFAV |
| | LNSVTPEDTAIYYCTRA | YYCQQRSNTFGPGTKVD |
| | QWLGGDYPYYYSMDVWG | IK |
| | QGTTVTVSS | (SEQ ID NO: 230) |
| | (SEQ ID NO: 229) | CDR1 |
| | CDR1 | (SEQ ID NO: 234)- |
| | (SEQ ID NO: 231)- | QSVSSYLA |
| | GDSVSSNSA | CDR2 |
| | CDR2 | (SEQ ID NO: 235)- |
| | (SEQ ID NO: 232)- | DASNRAT |
| | YYRSKWY | CDR3 |
| | CDR3 | (SEQ ID NO: 236)- |
| | (SEQ ID NO: 233)- | QQRSNT |
| | AQWLGGDYPYYYSMDV | |
| vorsetuzumab | QVQLVQSGAEVKKPGAS | DIVMTQSPDSLAVSLGE |
| | VKVSCKASGYTFTNYGM | RATINCRASKSVSTSGY |
| | NWVRQAPGQGLKWMGWI | SFMHWYQQKPGQPPKLL |
| | NTYTGEPTYADAFKGRV | IYLASNLESGVPDRFSG |
| | TMTRDTSISTAYMELSR | SGSGTDFTLTISSLQAE |
| | LRSDDTAVYYCARDYGD | DVAVYYCQHSREVPWTF |
| | YGMDYWGQGTTVTVSSA | GQGTKVEIKR |
| | (SEQ ID NO: 237) | (SEQ ID NO: 238) |
| | CDR1 | CDR1 |
| | (SEQ ID NO: 239)- | (SEQ ID NO: 242)- |
| | GYTFTNY | KSVSTSGYSFMH |
| | CDR2 | CDR2 |
| | (SEQ ID NO: 240)- | (SEQ ID NO: 243)- |
| | NTYTGE | LASNLES |
| | CDR3 | CDR3 |
| | (SEQ ID NO: 241)- | (SEQ ID NO: 244)- |
| | DYGDYGMDY | QHSREVPWT |
| polatuzumab | EVQLVESGGGLVQPGGS | DIQLTQSPSSLSASVGD |
| | LRLSCAASGYTFSSYWI | RVTITCKASQSVDYEGD |
| | EWVRQAPGKGLEWIGEI | SFLNWYQQKPGKAPKLL |
| | LPGGGDTNYNEIFKGRA | IYAASNLESGVPSRFSG |
| | TFSADTSKNTAYLQMNS | SGSGTDFTLTISSLQPE |
| | LRAEDTAVYYCTRRVPI | DFATYYCQQSNEDPLTF |
| | RLDYWGQGTLVTVSSA | GQGTKVEIKR |
| | (SEQ ID NO: 245) | (SEQ ID NO: 246) |
| | CDR1 | CDR1 |
| | (SEQ ID NO: 247)- | (SEQ ID NO: 250)- |
| | GYTFSSY | QSVDYEGDSFLN |
| | CDR2 | CDR2 |
| | (SEQ ID NO: 248)- | (SEQ ID NO: 251)- |
| | LPGGGD | AASNLES |
| | CDR3 | CDR3 |
| | (SEQ ID NO: 249)- | (SEQ ID NO: 252)- |
| | RVPIRLDY | QQSNEDPLT |
| galiximab | QVQLQESGPGLVKPSET | ESALTQPPSVSGAPGQK |
| | LSLTCAVSGGSISGGYG | VTISCTGSTSNIGGYDL |
| | WGWIRQPPGKGLEWIGS | HWYQQLPGTAPKLLIYD |
| | FYSSSGNTYYNPSLKSQ | INKRPSGISDRFSGSKS |
| | VTISTDTSKNQFSLKLN | GTAASLAITGLQTEDEA |
| | SMTAADTAVYYCVRDRL | DYYCQSYDSSLNAQVFG |
| | FSVVGMVYNNWFDVWGP | GGTRLTVLG |
| | GVLVTVSSA | (SEQ ID NO: 254) |
| | (SEQ ID NO: 253) | CDR1 |
| | CDR1 | (SEQ ID NO: 258)- |
| | (SEQ ID NO: 255)- | TSNIGGYDLH |
| | GGSISGGY | CDR2 |
| | CDR2 | (SEQ ID NO: 259)- |
| | (SEQ ID NO: 256)- | DINKRPS |
| | YSSSGN | CDR3 |
| | CDR3 | (SEQ ID NO: 260)- |
| | (SEQ ID NO: 257)- | QSYDSSLNAQV |
| | DRLFSVVGMVYNNWFD | |
| | V | |
| US | EVQLLESGGGLVQPGGS | DIQMTQSPSSLSASVGD |
| 20160046720 | LRLSCAASGFTFRSSAM | RVTITCRASQDISNYLA |
| | HWVRQAPGKGLKWVSSV | WFQQKPGKAPKSLIYTA |
| | SGSGAGTYYADSVKGRF | SSLQSGVPSKFSGSGSG |
| | TISRDNPKNTLYLQMNS | TDFTLTISSLQPEDFAT |
| | LRAEDTAVYYCVKEGGS | YYCQQYNLYPPTFGQGT |
| | RGFDYWGQGTLVTVSS | KVEIKR |
| | (SEQ ID NO: 261) | (SEQ ID NO: 262) |
| | CDR1 | CDR1 |
| | (SEQ ID NO: 263)- | (SEQ ID NO: 266)- |
| | GFTFRSS | QDISNYLA |
| | CDR2 | CDR2 |
| | (SEQ ID NO: 264)- | (SEQ ID NO: 267)- |
| | SVSGSGAGTYYADSVKG | YTASSLQSGVPSKFS |
| | CDR3 | CDR3 |
| | (SEQ ID NO: 265)- | (SEQ ID NO: 268)- |
| | EGGSRGFDY | QQYNLYPPT |

Antigen-binding sites that can bind to tumor associated antigen SLAMF7 can be identified by screening for binding to the amino acid sequence defined by SEQ ID NO:269.

SEQ ID NO: 269
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEEYIEEKKRVDICRETPNICPHSGENTEYDTIPHTNRTILKEDPA
NTVYSTVEIPKKMENPHSLLTMPDTPRLFAYENVI

Antigen-binding sites that can bind to tumor associated antigen CD38 can be identified by screening for binding to the amino acid sequence defined by SEQ ID NO:270.

SEQ ID NO: 270
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

Antigen-binding sites that can bind to tumor associated antigen CD138 can be identified by screening for binding to the amino acid sequence defined by SEQ ID NO:271.

SEQ ID NO: 271
MRRAALWLWLCALALSLQPALPQIVATNLPPEDQDGSGDDSDNFSGSGAG
ALQDITLSQQTPSTWKDTQLLTAIPTSPEPTGLEATAASTSTLPAGEGPK
EGEAVVLPEVEPGLTAREQEATPRPRETTQLPTTHLASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

Alternatively, Table 6 lists peptide sequences of heavy chain variable domains and light chain variable domains that, in combination, can bind to SLAMF7 (elotuzumab, azintuxizumab), CD138 (indatuximab), or CD38 (daratumumab, MOR202).

TABLE 6

| | Heavy chain | Light chain |
|---|---|---|
| | variable domain | variable domain |
| | amino acid | amino acid |
| Clones | sequence | sequence |
| elotuzumab | EVQLVESGGGLVQPGGS | DIQMTQSPSSLSASVGD |
| | LRLSCAASGFDFSRYWM | RVTITCKASQDVGIAVA |
| | SWVRQAPGKGLEWIGEI | WYQQKPGKVPKLLIYWA |
| | NPDSSTINYAPSLKDKF | STRHTGVPDRFSGSGSG |
| | IISRDNAKNSLYLQMNS | TDFTLTISSLQPEDVAT |
| | LRAEDTAVYYCARPDGN | YYCQQYSSYPYTFGQGT |
| | YWYFDVWGQGTLVTVSS | KVEIKR |
| | A | (SEQ ID NO: 273) |
| | (SEQ ID NO: 272) | CDR1 |
| | CDR1 | (SEQ ID NO: 277)- |
| | (SEQ ID NO: 274)- | QDVGIAVA |
| | GFDFSRY | CDR2 |
| | CDR2 | (SEQ ID NO: 278)- |
| | (SEQ ID NO: 275)- | WASTRHT |
| | NPDSST | CDR3 |
| | CDR3 | (SEQ ID NO: 279)- |
| | (SEQ ID NO: 276)- | QQYSSYPYT |
| | PDGNYWYFDV | |
| azintuxi- | EVQLVESGGGLVQPGGS | DVVMTQTPLSLSVTPGQ |
| zumab | LRLSCAASGFTFSDYYM | PASISCRSSQSLVHSNG |
| | AWVRQAPGKGLEWVASI | NTYLHWYLQKPGQSPQL |
| | NYDGSSTYYVDSVKGRF | LIYKVSNRFSGVPDRFS |
| | TISRDNAKNSLYLQMNS | GSGSGTDFTLKISRVEA |
| | LRAEDTAVYYCARDRGY | EDVGVYFCSQSTHVPPF |
| | YFDYWGQGTTVTVSSA | TFGGGTKVEIKR |
| | (SEQ ID NO: 280) | (SEQ ID NO: 281) |
| | CDR1 | CDR1 |
| | (SEQ ID NO: 282)- | (SEQ ID NO: 285)- |
| | GFTFSDYYMA | CRSSQSLVHSNGNTYLH |
| | CDR2 | CDR2 |
| | (SEQ ID NO: 283)- | (SEQ ID NO: 286)- |
| | SINYDGSSTYYVDSVKG | KVSNRFS |
| | RFTISRDNA | CDR3 |
| | CDR3 | (SEQ ID NO: 287)- |
| | (SEQ ID NO: 284)- | SQSTHVPPFT |
| | DRGYYFDY | |
| indatuximab | QVQLQQSGSELMMPGAS | DIQMTQSTSSLSASLGD |
| | VKISCKATGYTFSNYWI | RVTISCSASQGINNYLN |
| | EWVKQRPGHGLEWIGEI | WYQQKPDGTVELLIYYT |
| | LPGTGRTIYNEKFKGKA | STLQSGVPSRFSGSGSG |
| | TFTADISSNTVQMQLSS | TDYSLTISNLEPEDIGT |
| | LTSEDSAVYYCARRDYY | YYCQQYSKLPRTFGGGT |
| | GNFYYAMDYWGQGTSVT | KLEIKR |
| | VSSA | (SEQ ID NO: 289) |
| | (SEQ ID NO: 288) | CDR1 |
| | CDR1 | (SEQ ID NO: 293)- |
| | (SEQ ID NO: 290)- | QGINNYLN |
| | GYTFSNY | CDR2 |
| | CDR2 | (SEQ ID NO: 294)- |
| | (SEQ ID NO: 291)- | YTSTLQS |
| | LPGTGR | CDR3 |
| | CDR3 | (SEQ ID NO: 295)- |
| | (SEQ ID NO: 292)- | QQYSKLPRT |
| | RDYYGNFYYAMDY | |
| daratumumab | EVQLLESGGGLVQPGGS | EIVLTQSPATLSLSPGE |
| | LRLSCAVSGFTFNSFAM | RATLSCRASQSVSSYLA |
| | SWVRQAPGKGLEWVSAI | WYQQKPGQAPRLLIYDA |
| | SGSGGGTYYADSVKGRF | SNRATGIPARFSGSGSG |
| | TISRDNSKNTLYLQMNS | TDFTLTISSLEPEDFAV |
| | LRAEDTAVYFCAKDKIL | YYCQQRSNWPPTFGQGT |
| | WFGEPVFDYWGQGTLVT | KVEIKR |
| | VSSA | (SEQ ID NO: 297) |
| | (SEQ ID NO: 296) | CDR1 |
| | CDR1 | (SEQ ID NO: 301)- |
| | (SEQ ID NO: 298)- | QSVSSYLA |
| | GFTFNSF | CDR2 |
| | CDR2 | (SEQ ID NO: 302)- |
| | (SEQ ID NO: 299)- | DASNRAT |
| | SGSGGG | CDR3 |
| | CDR3 | (SEQ ID NO: 303)- |
| | (SEQ ID NO: 300)- | QQRSNWPPT |
| | DKILWFGEPVFDY | |
| MOR202 | QVQLVESGGGLVQPGGS | DIELTQPPSVSVAPGQT |
| | LRLSCAASGFTFSSYYM | ARISCSGDNLRHYYWWY |
| | NWVRQAPGKGLEWVSGI | QQKPGQAPVLVIYGDSK |
| | SGDPSNTYYADSVKGRF | RPSGIPERFSGSNSGNT |
| | TISRDNSKNTLYLQMNS | ATLTISGTQAEDEADYY |
| | LRAEDTAVYYCARDLPL | CQTYTGGASLVFGGGTK |
| | VYTGFAYWGQGTLVTVS | LTVLGQ |
| | S | (SEQ ID NO: 305) |
| | (SEQ ID NO: 304) | CDR1 |
| | CDR1 | (SEQ ID NO: 309)- |
| | (SEQ ID NO: 306)- | SGDNLRHYYW |
| | GFTFSSYYMN | CDR2 |
| | CDR2 | (SEQ ID NO: 310)- |
| | (SEQ ID NO: 307)- | GDSKRPS |
| | GISGDPSNTYYADSVKG | CDR3 |
| | RFTISRDNS | (SEQ ID NO: 311)- |
| | CDR3 | QTYTGGASLV |
| | (SEQ ID NO: 308)- | |
| | DLPLVYTGFAY | |

Antigen-binding sites that can bind to tumor associated antigen TRBC1 can be identified by screening for binding to the amino acid sequence defined by SEQ ID NO:312.

SEQ ID NO: 312
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

Antigen-binding sites that can bind to tumor associated antigen TRBC2 can be identified by screening for binding to the amino acid sequence defined by SEQ ID NO:313.

SEQ ID NO: 313
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

Antigen-binding sites that bind to different tumor associated antigens can be routinely identified by screening for binding to the amino acid sequence of each antigen. For example, antigen-binding sites that bind to LILRB2 can be routinely identified by screening for binding to the amino acid sequence of LILRB2 as defined by SEQ ID NO:314.

SEQ ID NO: 314
MTPIVTVLICLGLSLGPRTHVQTGTIPKPTLWAEPDSVITQGSPVTLSCQ
GSLEAQEYRLYREKKSASWITRIRPELVKNGQFHIPSITWEHTGRYGCQY
YSRARWSELSDPLVLVMTGAYPKPTLSAQPSPVVTSGGRVTLQCESQVAF
GGFILCKEGEEEHPQCLNSQPHARGSSRAIFSVGPVSPNRRWSHRCYGYD
LNSPYVWSSPSDLLELLVPGVSKKPSLSVQPGPVVAPGESLTLQCVSDVG
YDRFVLYKEGERDLRQLPGRQPQAGLSQANFTLGPVSRSYGGQYRCYGAH
NLSSECSAPSDPLDILITGQIRGTPFISVQPGPTVASGENVTLLCQSWRQ
FHTFLLTKAGAADAPLRLRSIHEYPKYQAEFPMSPVTSAHAGTYRCYGSL
NSDPYLLSHPSEPLELVVSGPSMGSSPPPTGPISTPAGPEDQPLTPTGSD
PQSGLGRHLGVVIGILVAVVLLLLLLLLLFLILRHRRQGKHWTSTQRKAD
FQHPAGAVGPEPTDRGLQWRSSPAADAQEENLYAAVKDTQPEDGVEMDTR
AAASEAPQDVTYAQLHSLTLRRKATEPPPSQEREPPAEPSIYATLAIH

Antigen-binding sites that bind to LILRB1 can be routinely identified by screening for binding to the amino acid sequence of LILRB1 as defined by SEQ ID NO:315.

SEQ ID NO: 315
MTPILTVLICLGLSLGPRTHVQAGHLPKPTLWAEPGSVITQGSPVTLRCQ
GGQETQEYRLYREKKTALWITRIPQELVKKGQFPIPSITWEHAGRYRCYY
GSDTAGRSESSDPLELVVTGAYIKPTLSAQPSPVVNSGGNVILQCDSQVA
FDGFSLCKEGEDEHPQCLNSQPHARGSSRAIFSVGPVSPSRRWWYRCYAY
DSNSPYEWSLPSDLLELLVLGVSKKPSLSVQPGPIVAPEETLTLQCGSDA
GYNRFVLYKDGERDFLQLAGAQPQAGLSQANFTLGPVSRSYGGQYRCYGA
HNLSSEWSAPSDPLDILIAGQFYDRVSLSVQPGPTVASGENVTLLCQSQG
WMQTFLLTKEGAADDPWRLRSTYQSQKYQAEFPMGPVTSAHAGTYRCYGS
QSSKPYLLTHPSDPLELVVSGPSGGPSSPTTGPTSTSGPEDQPLTPTGSD
PQSGLGRHLGVVIGILVAVILLLLLLLLLFLILRHRRQGKHWTSTQRKAD
FQHPAGAVGPEPTDRGLQWRSSPAADAQEENLYAAVKHTQPEDGVEMDTR
SPHDEDPQAVTYAEVKHSRPRREMASPPSPLSGEFLDTKDRQAEEDRQMD
TEAAASEAPQDVTYAQLHSLTLRREATEPPPSQEGPSPAVPSIYATLAIH

Antigen-binding sites that bind to LILRB3 can be routinely identified by screening for binding to the amino acid sequence of LILRB3 as defined by SEQ ID NO:316.

SEQ ID NO: 316
MTPALTALLCLGLSLGPRTRVQAGPFPKPTLWAEPGSVISWGSPVTIWCQ
GSQEAQEYRLHKEGSPEPLDRNNPLEPKNKARFSIPSMTEHHAGRYRCHY
YSSAGWSEPSDPLEMVMTGAYSKPTLSALPSPVVASGGNMTLRCGSQKGY
HHFVLMKEGEHQLPRTLDSQQLHSRGFQALFPVGPVTPSHRWRFTCYYYY
TNTPWVWSHPSDPLEILPSGVSRKPSLLTLQGPVLAPGQSLTLQCGSDVG
YNRFVLYKEGERDFLQRPGQQPQAGLSQANFTLGPVSPSNGGQYRCYGAH
NLSSEWSAPSDPLNILMAGQIYDTVSLSAQPGPTVASGENVTLLCQSWWQ
FDTFLLTKEGAAHPPLRLRSMYGAHKYQAEFPMSPVTSAHAGTYRCYGSY
SSNPHLLSHPSEPLELVVSGHSGGSSLPPTGPPSTPGLGRYLEVLIGVSV
AFVLLLFLLLFLLLRRQRHSKHRTSDQRKTDFQRPAGAAETEPKDRGLLR
RSSPAADVQEENLYAAVKDTQSEDRVELDSQSPHDEDPQAVTYAPVKHSS
PRREMASPPSSLSGEFLDTKDRQVEEDRQMDTEAAASEASQDVTYAQLHS
LTLRRKATEPPPSQEGEPPAEPSIYATLAIH

Antigen-binding sites that bind to LILRB4 can be routinely identified by screening for binding to the amino acid sequence of LILRB4 as defined by SEQ ID NO:317.

SEQ ID NO: 317
MIPTFTALLCLGLSLGPRTHMQAGPLPKPTLWAEPGSVISWGNSVTIWCQ
GTLEAREYRLDKEESPAPWDRQNPLEPKNKARFSIPSMTEDYAGRYRCYY
RSPVGWSQPSDPLELVMTGAYSKPTLSALPSPLVTSGKSVTLLCQSRSPM
DTFLLIKERAAHPLLHLRSEHGAQQHQAEFPMSPVTSVHGGTYRCFSSHG
FSHYLLSHPSDPLELIVSGSLEDPRPSPTRSVSTAAGPEDQPLMPTGSVP
HSGLRRHWEVLIGVLVVSILLLSLLLFLLLQHWRQGKHRTLAQRQADFQR
PPGAAEPEPKDGGLQRRSSPAADVQGENFCAAVKNTQPEDGVEMDTRQSP
HDEDPQAVTYAKVKHSRPRREMASPPSPLSGEFLDTKDRQAEEDRQMDTE
AAASEAPQDVTYAQLHSFTLRQKATEPPPSQEGASPAEPSVYATLAIH

Antigen-binding sites that bind to LILRB5 can be routinely identified by screening for binding to the amino acid sequence of LILRB5 as defined by SEQ ID NO:318.

SEQ ID NO: 318
MTLTLSVLICLGLSVGPRTCVQAGTLPKPTLWAEPASVIARGKPVTLWCQ
GPLETEEYRLDKEGLPWARKRQNPLEPGAKAKFHIPSTVYDSAGRYRCYY
ETPAGWSEPSDPLELVATGFYAEPTLLALPSPVVASGGNVTLQCDTLDGL
LTFVLVEEEQKLPRTLYSQKLPKGPSQALFPVGPVTPSCRWRFRCYYYYR
KNPQVWSNPSDLLEILVPGVSRKPSLLIPQGSVVARGGSLTLQCRSDVGY
DIFVLYKEGEHDLVQGSGQQPQAGLSQANFTLGPVSRSHGGQYRCYGAHN
LSPRWSAPSDPLDILIAGLIPDIPALSVQPGPKVASGENVTLLCQSWHQI
DTFFLTKEGAAHPPLCLKSKYQSYRHQAEFSMSPVTSAQGGTYRCYSAIR
SYPYLLSSPSYPQELVVSGPSGDPSLSPTGSTPTPGPEDQPLTPTGLDPQ
SGLGRHLGVVTGVSVAFVLLLFLLLFLLLRHRHQSKHRTSAHFYRPAGAA
GPEPKDQGLQKRASPVADIQEEILNAAVKDTQPKDGVEMDARAAASEAPQ
DVTYAQLHSLTLRREATEPPPSQEREPPAEPSIYAPLAIH

Antigen-binding sites that bind to LILRA1 can be routinely identified by screening for binding to the amino acid sequence of LILRA1 as defined by SEQ ID NO:319.

SEQ ID NO: 319
MTPIVTVLICLRLSLGPRTHVQAGTLPKPTLWAEPGSVITQGSPVTLWCQ
GILETQEYRLYREKKTAPWITRIPQEIVKKGQFPIPSITWEHTGRYRCFY
GSHTAGWSEPSDPLELVVTGAYIKPTLSALPSPVVTSGGNVTLHCVSQVA
FGSFILCKEGEDEHPQCLNSQPRTHGWSRAIFSVGPVSPSRRWSYRCYAY
DSNSPHVWSLPSDLLELLVLGVSKKPSLSVQPGPIVAPGESLTLQCVSDV
SYDRFVLYKEGERDFLQLPGPQPQAGLSQANFTLGPVSRSYGGQYRCSGA
YNLSSEWSAPSDPLDILIAGQFRGRPFISVHPGPTVASGENVTLLCQSWG
PFHTFLLTKAGAADAPLRLRSIHEYPKYQAEFPMSPVTSAHSGTYRCYGS
LSSNPYLLSHPSDSLELMVSGAAETLSPPQNKSDSKAGAANTLSPSQNKT
ASHPQDYTVENLIRMGIAGLVLVVLGILLFEAQHSQRSL

Antigen-binding sites that bind to LILRA2 can be routinely identified by screening for binding to the amino acid sequence of LILRA2 as defined by SEQ ID NO:320.

SEQ ID NO: 320
MTPILTVLICLGLSLGPRTHVQAGHLPKPTLWAEPGSVIIQGSPVTLRCQ
GSLQAEEYHLYRENKSASWVRRIQEPGKNGQFPIPSITWEHAGRYHCQYY
SHNHSSEYSDPLELVVTGAYSKPTLSALPSPVVTLGGNVTLQCVSQVAFD
GFILCKEGEDEHPQRLNSHSHARGWSWAIFSVGPVSPSRRWSYRCYAYDS
NSPYVWSLPSDLLELLVPGVSKKPSLSVQPGPMVAPGESLTLQCVSDVGY
DRFVLYKEGERDFLQRPGWQPQAGLSQANFTLGPVSPSHGGQYRCYSAHN
LSSEWSAPSDPLDILITGQFYDRPSLSVQPVPTVAPGKNVTLLCQSRGQF
HTFLLTKEGAGHPPLHLRSEHQAQQNQAEFRMGPVTSAHVGTYRCYSSLS
SNPYLLSLPSDPLELVVSEAAETLSPSQNKTDSTTTSLGQHPQDYTVENL
IRMGVAGLVLVVLGILLFEAQHSQRSLQDAAGR

Antigen-binding sites that bind to LILRA3 can be routinely identified by screening for binding to the amino acid sequence of LILRA3 as defined by SEQ ID NO:321.

SEQ ID NO: 321
MTPILTVLICLGLSLDPRTHVQAGPLPKPTLWAEPGSVITQGSPVTLRCQ
GSLETQEYHLYREKKTALWITRIPQELVKKGQFPILSITWEHAGRYCCIY
GSHTAGLSESSDPLELVVTGAYSKPTLSALPSPVVTSGGNVTIQCDSQVA
FDGFILCKEGEDEHPQCLNSHSHARGSSRAIFSVGPVSPSRRWSYRCYGY
DSRAPYVWSLPSDLLGLLVPGVSKKPSLSVQPGPVVAPGEKLTFQCGSDA
GYDRFVLYKEWGRDFLQRPGRQPQAGLSQANFTLGPVSRSYGGQYTCSGA
YNLSSEWSAPSDPLDILITGQIRARPFLSVRPGPTVASGENVTLLCQSQG
GMHTFLLTKEGAADSPLRLKSKRQSHKYQAEFPMSPVTSAHAGTYRCYGS
LSSNPYLLTHPSDPLELVVSGAAETLSPPQNKSDSKAGE

Antigen-binding sites that bind to LILRA4 can be routinely identified by screening for binding to the amino acid sequence of LILRA4 as defined by SEQ ID NO:322.

SEQ ID NO: 322
MTLILTSLLFFGLSLGPRTRVQAENLPKPILWAEPGPVITWHNPVTIWCQ
GTLEAQGYRLDKEGNSMSRHILKTLESENKVKLSIPSMMWEHAGRYHCYY
QSPAGWSEPSDPLELVVTAYSRPTLSALPSPVVTSGVNVTLRCASRLGLG
RFTLIEEGDHRLSWTLNSHQHNHGKFQALFPMGPLTFSNRGTFRCYGYEN
NTPYVWSEPSDPLQLLVSGVSRKPSLLTLQGPVVTPGENLTLQCGSDVGY
IRYTLYKEGADGLPQRPGRQPQAGLSQANFTLSPVSRSYGGQYRCYGAHN
VSSEWSAPSDPLDILIAGQISDRPSLSVQPGPTVTSGEKVTLLCQSWDPM
FTFLLTKEGAAHPPLRLRSMYGAHKYQAEFPMSPVTSAHAGTYRCYGSRS
SNPYLLSHPSEPLELVVSGATETLNPAQKKSDSKTAPHLQDYTVENLIRM
GVAGLVLLFLGILLFEAQHSQRSPPRCSQEANSRKDNAPFRVVEPWEQI

Antigen-binding sites that bind to LILRA5 can be routinely identified by screening for binding to the amino acid sequence of LILRA5 as defined by SEQ ID NO:323.

SEQ ID NO: 323
MAPWSHPSAQLQPVGGDAVSPALMVLLCLGLSLGPRTHVQAGNLSKATLW
AEPGSVISRGNSVTIRCQGTLEAQEYRLVKEGSPEPWDTQNPLEPKNKAR
FSIPSMTEHHAGRYRCYYYSPAGWSEPSDPLELVVTGFYNKPTLSALPSP
VVTSGENVTLQCGSRLRFDRFILTEEGDHKLSWTLDSQLTPSGQFQALFP
VGPVTPSHRWMLRCYGSRRHILQVWSEPSDLLEIPVSGAADNLSPSQNKS
DSGTASHLQDYAVENLIRMGMAGLILVVLGILIFQDWHSQRSPQAAAGR

Antigen-binding sites that bind to LILRA6 can be routinely identified by screening for binding to the amino acid sequence of LILRA6 as defined by SEQ ID NO:324.

SEQ ID NO: 324
MTPALTALLCLGLSLGPRTRVQAGPFPKPTLWAEPGSVISWGSPVTIWCQ
GSLEAQEYQLDKEGSPEPLDRNNPLEPKNKARFSIPSMTQHHAGRYRCHY
YSSAGWSEPSDPLELVMTGFYNKPTLSALPSPVVASGGNMTLRCGSQKGY
HHFVLMKEGEHQLPRTLDSQQLHSGGFQALFPVGPVTPSHRWRFTCYYYY
TNTPRVWSHPSDPLEILPSGVSRKPSLLTLQGPVLAPGQSLTLQCGSDVG
YDRFVLYKEGERDFLQRPGQQPQAGLSQANFTLGPVSPSHGGQYRCYGAH
NLSSEWSAPSDPLNILMAGQIYDTVSLSAQPGPTVASGENVTLLCQSRGY
FDTFLLTKEGAAHPPLRLRSMYGAHKYQAEFPMSPVTSAHAGTYRCYGSY
SSNPHLLSFPSEPLELMVSGHSGGSSLPPTGPPSTPASHAKDYTVENLIR
MGMAGLVLVFLGILLFEAQHSQRNPQDAAGR

Table 7 lists examples of peptide sequences of heavy chain variable domains that by itself or in combination with light chain variable domains, can bind to each of T_{reg} associated antigens.

TABLE 7

| | Heavy chain | Light chain |
|---|---|---|
| | variable domain | variable domain |
| Examples* | amino acid sequence | amino acid sequence |
| Anti-CD7 | MDVQLQESGGGSVQAGGSLR | |
| (US20170226204A1) | LSCPASGYTFSHYCMGWNRQ | |
| | APGKEREEVATIDTDDTPTYA | |
| | DSVMGRFTISRDNANNALYL | |
| | QMNDLKPEDTSMYYCAIWM | |
| | KLRGSCHDRRLEVRGQGTQV | |
| | TVSIN (SEQ ID NO: 325) | |
| | CDR1 (SEQ ID NO: 326)- | |
| | GYTFSHYCM | |
| | CDR2 (SEQ ID NO: 327)- | |
| | TIDTDDTPT | |
| | CDR3 (SEQ ID NO: 328)- | |
| | AIWMKLRGSCHDRRLE | |
| Anti-CD7 | MDVQLQESGGGSVQAGGSLR | |
| (US20170226204A1) | LSCAASGYTHSSYCMAWFRQ | |
| | APGREREGVASIDSDGTTSYA | |
| | DSVKGRFTISQDNAKNTLYL | |
| | QMNSLKPEDTAMYYCAARF | |
| | GPMGCVDLSTLSFGHWGQGT | |
| | QVTVSIT (SEQ ID NO: 329) | |
| | CDR1 (SEQ ID NO: 330)- | |
| | GYTHSSYCM | |
| | CDR2 (SEQ ID NO: 331)- | |
| | SIDSDGTTS | |
| | CDR3 (SEQ ID NO: 332)- | |
| | AARFGPMGCVDLSTLSFGH | |
| Anti-CTLA4 | QVQLVESGGGVVQPGRSLRL | EIVLTQSPGTLSLSPGERATL |
| (ipilimumab) | SCAASGFTFSSYTMHWVRQA | SCRASQSVGSSYLAWYQQK |
| | PGKGLEWVTFISYDGNNKYY | PGQAPRLLIYGAFSRATGIP |
| | ADSVKGRFTISRDNSKNTLYL | DRFSGSGSGTDFTLTISRLEP |
| | QMNSLRAEDTAIYYCARTGW | EDFAVYYCQQYGSSPWTFG |
| | LGPFDYWGQGTLVTVSS | QGTKVEIK (SEQ ID NO: 334) |
| | (SEQ ID NO: 333) | CDR1 (SEQ ID NO: 338)- |
| | CDR1 (SEQ ID NO: 335)- | QSVGSSYLA |
| | GFTFSSY | CDR2 (SEQ ID NO: 339)- |
| | CDR2 (SEQ ID NO: 336)- | GAFSRAT |
| | SYDGNN | CDR3 (SEQ ID NO: 340)- |
| | CDR3 (SEQ ID NO: 337)- | QQYGSSPWT |
| | TGWLGPFDY | |
| Anti-CTLA4 | QVQLVESGGGVVQPGRSLRL | DIQMTQSPSSLSASVGDRVT |
| (tremelimumab) | SCAASGFTFSSYGMHWVRQA | ITCRASQSINSYLDWYQQKP |
| | PGKGLEWVAVIWYDGSNKY | GKAPKLLIYAASSLQSGVPS |
| | YADSVKGRFTISRDNSKNTLY | RFSGSGSGTDFTLTISSLQPE |
| | LQMNSLRAEDTAVYYCARDP | DFATYYCQQYYSTPFTFGP |
| | RGATLYYYYYGMDVWGQGT | GTKVEIKRTVAAPSVFIFPPS |
| | TVTVSSASTKGPSVFPLAPCS | DEQLKSGTASVVCLLNNFY |
| | RSTSESTAALGCLVKDYFPEP | PREAKVQWKVDNALQSGN |
| | VTVSWNSGALTSGVHTFPAV | SQESVTEQDSKDSTYSLSST |
| | LQSSGLYSLSSVVTVPSSNFG | LTLSKADYEKHKVYACEVT |
| | TQTYTCNVDHKPSNTKVDKT | HQGLSSPVTKSFNRGEC |
| | VERKCCVECPPCPAPPVAGPS | (SEQ ID NO: 342) |
| | VFLFPPKPKDTLMISRTPEVT | CDR1 (SEQ ID NO: 346)- |
| | CVVVDVSHEDPEVQFNWYV | QSINSYLD |
| | DGVEVHNAKTKPREEQFNST | CDR2 (SEQ ID NO: 347)- |
| | FRVVSVLTVVHQDWLNGKE | AASSLQS |
| | YKCKVSNKGLPAPIEKTISKT | CDR3 (SEQ ID NO: 348)- |
| | KGQPREPQVYTLPPSREEMTK | QQYYSTPFT |
| | NQVSLTCLVKGFYPSDIAVE | |
| | WESNGQPENNYKTTPPMLDS | |
| | DGSFFLYSKLTVDKSRWQQG | |
| | NVFSCSVMHEALHNHYTQKS | |
| | LSLSPGK (SEQ ID NO: 341) | |
| | CDR1 (SEQ ID NO: 343)- | |
| | GFTFSSY | |
| | CDR2 (SEQ ID NO: 344)- | |
| | WYDGSN | |
| | CDR3 (SEQ ID NO: 345)- | |
| | DPRGATLYYYYYGMDV | |
| Anti-CX3CR1 | EVQLVESGGGSVQAGESLRL | |
| (WO2013130381A1) | SCAASGSIFSSNAMAWYRQA | |
| | PGKQRDLVAGINSVGITKYA | |
| | DSVKGRFTISRDNAKNTVYL | |
| | QMNSLKPEDTAVYYCTSDPR | |
| | RGWDTRYWGQGTQVTVSS | |
| | (SEQ ID NO: 349) | |
| | CDR1 (SEQ ID NO: 350)- | |
| | GSIFSSNAMA | |
| | CDR2 (SEQ ID NO: 351)- | |
| | AINSVGVTK | |
| | CDR3 (SEQ ID NO: 352)- | |
| | DPRRGWDTRY | |
| Anti-CX3CR1 | VQLVESGGGLVQPGGSLRLS | |
| (WO2013130381A1) | CAASGSIFSSTAMAWYRQAP | |
| | GKRRDLVAAISSVGVTKYAD | |
| | SVKGRFTISRDNSKNTVYLQ | |
| | MNSLRPEDTAVYYCTSDPRR | |
| | GWDTRYWGQGTLVTVSS | |
| | (SEQ ID NO: 353) | |
| | CDR1 (SEQ ID NO: 354)- | |
| | GSIFSSTAMA | |
| | CDR2 (SEQ ID NO: 356)- | |
| | AISSVGVTK | |
| | CDR3 (SEQ ID NO: 357)- | |
| | DPRRGWDTRY | |
| Anti-ENTPD1 | EVQLVESGGDLVKPGGSLKL | DVVMTQTPLSLPVSLGDQA |
| (WO2016073845A1) | SCAAFGFTFSRYGMSWVRQT | SISCRSSQSLLHSNGNTYLH |
| | PDKRLEWVATITSGGIYTYYP | WYLQKPGQSPKLLIYKVSN |
| | DSVKGRFTISRDNAKNTLYLQ | RFSGVPDRFSGSGSGTDFTL |
| | MSSLKSEETAMYYCARHGQF | KISRVEAEDLGVYFCSQSTH |
| | GDYYGMDYWGQGTSVTVSS | VPYTFGGGTKLEIK (SEQ ID |
| | (SEQ ID NO: 358) | NO: 359) |
| | CDR1 (SEQ ID NO: 360)- | CDR1 (SEQ ID NO: 363)- |
| | GFTFSRYGMS | RSSQSLLHSNGNTYLH |
| | CDR2 (SEQ ID NO: 361)- | CDR2 (SEQ ID NO: 364)- |
| | TITSGGIYTYYPDSVKG | KVSNRFS |
| | CDR3 (SEQ ID NO: 362)- | CDR3 (SEQ ID NO: 365)- |
| | HGQFGDYYGMDY | SQSTHVPYT |
| Anti-ENTPD1 | QVQLVQSGSELKKPGASVKV | DIQMTQSPSSLSASVGDRVT |
| (WO2017157948A1) | SCKASGYTFTHYGMNWVRQ | ITCRASENIYSYFSWYQQKP |
| | APGQGLKWMGWINTYTGEP | GKAPKLLIYTAKTLAEGVPS |
| | TYADDFKGRFVFSLDTSVSTA | RFSGSGSGTDFTLTISSLQPE |
| | YLQISSLKAEDTAVYYCARR | DFATYYCQHHYVTPYTFGG |
| | RYEGNYVFYYFDYWGQGTT | GTKVEIK (SEQ ID NO: 367) |
| | VTVSS (SEQ ID NO: 366) | CDR1 (SEQ ID NO: 371)- |
| | CDR1 (SEQ ID NO: 368)- | RASENIYSYFS |
| | GYTFTHYG | CDR2 (SEQ ID NO: 372)- |
| | CDR2 (SEQ ID NO: 369)- | TAKTLAE |
| | NTYTGEP | CDR3 (SEQ ID NO: 373)- |
| | CDR3 (SEQ ID NO: 370)- | QHHYVTPYT |
| | ARRRYEGNYVFYYFDY | |
| Anti-HAVCR2 | EVQLLESGGGLVQPGGSLRLS | DIQMTQSPSSLSASVGDRVT |
| (WO2016161270A1) | CAAASGFTFSSYDMSWVRQA | ITCRASQSIRRYLNWYHQKP |
| | PGKGLDWVSTISGGGTYTYY | GKAPKLLIYGASTLQSGVPS |
| | QDSVKGRFTISRDNSKNTLYL | RFSGSGSGTDFTLTISSLQPE |
| | QMNSLRAEDTAVYYCASMD | DFAVYYCQQSHSAPLTFGG |
| | YWGQGTTVTVSSA (SEQ ID | GTKVEIKR (SEQ ID NO: 375) |
| | NO: 374) | CDR1 (SEQ ID NO: 379)- |
| | CDR1 (SEQ ID NO: 376)- | RASQSIRRYLN |
| | SGFTFSSYD | CDR2 (SEQ ID NO: 380)- |
| | CDR2 (SEQ ID NO: 377)- | GASTLQS |
| | SGGGTYT | CDR3 (SEQ ID NO: 381)- |
| | CDR3 (SEQ ID NO: 378)- | QQSHSAPLT |
| | ASMDY | |
| Anti-HAVCR2 | QVQLQQPGAELVKPGASVK | DIVLTQSPASLAVSLGQRAT |
| (US20170190777A1) | MSCKASGYTFTSYNMHWIKQ | ISCRASESVEYYGTSLMQW |
| | TPGQGLEWIGDIYPGNGDTSY | YQQKPGQPPKLLIYAASNV |
| | NQKFKGKATLTADKSSSTVY | ESGVPARFSGSGSGTDFSLN |
| | MQLSSLTSEDSAVYYCARVG | IHPVEEDDIAIYFCQQSRKD |
| | GAFPMDYWGQGTSVTVSS | PSTFGGGTKLEIK (SEQ ID |
| | (SEQ ID NO: 382) | NO: 383) |
| | CDR1 (SEQ ID NO: 384)- | CDR1 (SEQ ID NO: 387)- |
| | SYNMH | RASESVEYYGTSLMQ |
| | CDR2 (SEQ ID NO: 385)- | CDR2 (SEQ ID NO: 388)- |
| | DIYPGNGDTSYNQKFKG | AASNVES |
| | CDR3 (SEQ ID NO: 386)- | CDR3 (SEQ ID NO: 389)- |
| | VGGAFPMDY | QQSRKDPST |
| Anti-PDCD1LG2 | QVQLVQSGAEVKKPGASVKV | DIVMTQSPAFLSVTPGEKVT |
| (US20160137731A1) | SCKASGYTFTGYTMHWVRQ | ITCKSSQSLLNSGNQKNYLT |
| | APGQGLEWIGYINPRSGYTEY | WYQQKPGQPPKLLIYWAST |
| | NQKFKDRTTLTADKSTSTAY | RESGVPDRFSGSGSGTDFTL |
| | MELSSLRSEDTAVYYCARPW | TISSLQAEDVAVYYCQNDY |
| | FAYWGQGTLVTVSS (SEQ ID | SYPLTFGQGTKLEIK (SEQ ID |
| | NO: 390) | NO: 391) |
| | CDR1 (SEQ ID NO: 392)- | CDR1 (SEQ ID NO: 395)- |
| | GYTFTGYT | KSSQSLLNSGNQKNYLT |
| | CDR2 (SEQ ID NO: 393)- | CDR2 (SEQ ID NO: 396)- |
| | NPRSGYT | WASTRES |
| | CDR3 (SEQ ID NO: 394)- | CDR3 (SEQ ID NO: 397)- |
| | ARPWFAY | QNDYSYPLT |
| Anti-PDCD1LG2 | MNFGLSLIFLALILKGVQCEV | DIVMTQSPSSLATSVGQRVT |
| (WO2017053250A1) | QLVESGGDLVKSGGSLKLSC | MSCKSSQNLLYSTDQKNYL |
| | AASGFIFSSFGMSWVRQTPDK | AWFQQKPGQSPKLLLYFASI |
| | RLEWVATISSGGRNIYYLDSV | RESGVPDRFIGSGSGTDFTL |
| | KGRFTISRDNVKNILYLQMSG | TISSVQAEDLADYFCQQHY |
| | LKSEDSAMYYCAREGHYALD | NTPPTFGGGTRLEIK (SEQ ID |
| | YCGQGTSVTVSS (SEQ ID NO: | NO: 399) |
| | 398) | CDR1 (SEQ ID NO: 403)- |
| | CDR1 (SEQ ID NO: 400)- | KSSQNLLYSTDQKNYLA |
| | SFGMS | CDR2 (SEQ ID NO: 404)- |
| | CDR2 (SEQ ID NO: 401)- | FASIRES |
| | TISSGGRNIYYLDSVKG | CDR3 (SEQ ID NO: 405)- |
| | CDR3 (SEQ ID NO: 402)- | QQHYNTPPT |
| | EGHYALDY | |
| Anti-TIGIT | EVQLVQSGSDLKKPGASVRV | DIQLTQSPTFLSASVGDRVTI |
| (US20170088613A1) | SCKASGYTFTSYPMNWVRQA | TCRASQVISSSLAWYQQNP |
| | PGHGLEWMGWINTNTGNPT | GKAPKLLIYAASTLQSGVPS |
| | YVQGFTGRFVFSLDTSVNTA | RFSGSGSGTEFTLTISSLQPE |
| | YLQISSLKAEDTAVYFCARTG | DFVTYYCQHLHGYPSNFGQ |
| | GHTYDSYAFDVWGQGTMVT | GTKVEIK (SEQ ID NO: 407) |
| | VSS (SEQ ID NO: 406) | CDR1 (SEQ ID NO: 411)- |
| | CDR1 (SEQ ID NO: 408)- | RASQVISSSLA |
| | SYPMN | CDR2 (SEQ ID NO: 412)- |
| | CDR2 (SEQ ID NO: 409)- | AASTLQS |
| | WINTNTGNPTYVQGFTG | CDR3 (SEQ ID NO: 413)- |
| | CDR3 (SEQ ID NO: 410)- | QHLHGYPSN |
| | TGGHTYDSYAFDV | |
| Anti-TIGIT | DVQLQESGPGLVKPSQSLSLT | DIVMTQSHKFMSTSVGDRV |
| (US20160376365A1) | CTVTGYSITSDYAWNWVRQF | SITCKASQDVSTAVAWYQQ |
| | PGNKLEWMGYISYSGSTSYN | KPGQSPKLLIYSASYRYTGV |
| | PSLRSRISITRDTSKNQFFLQL | PDRFTGSGSGTDFTFTISSVQ |
| | NSVTTEDTATYYCARRQVGL | AEDLAVYYCQQHYSTPWTF |
| | GFAYWGQGTLVTVSS (SEQ | G (SEQ ID NO: 415) |
| | ID NO: 414) | CDR1 (SEQ ID NO: 419)- |
| | CDR1 (SEQ ID NO: 416)- | KASQDVSTAVA |
| | TSDYAWN | CDR2 (SEQ ID NO: 420)- |
| | CDR2 (SEQ ID NO: 417)- | SASYRYT |
| | YISYSGSTSYNPSLRS | CDR3 (SEQ ID NO: 421)- |
| | CDR3 (SEQ ID NO: 418)- | QQHYSTP |
| | ARRQVGLGFAY | |
| Anti-TNFRSF4 | EVQLVQSGAEVKKPGASVKV | DIQMTQSPSSLSASVGDRVT |
| (pogalizumab) | SCKASGYTFTDSYMSWVRQA | ITCRASQDISNYLNWYQQK |
| | PGQGLEWIGDMYPDNGDSSY | PGKAPKLLIYYTSRLRSGVP |
| | NQKFRERVTITRDTSTSTAYL | SRFSGSGSGTDFTLTISSLQP |
| | ELSSLRSEDTAVYYCVLAPR | EDFATYYCQQGHTLPPTFG |
| | WYFSVWGQGTLVTVSSASTK | QGTKVEIKRTVAAPSVFIFP |
| | GPSVFPLAPSSKSTSGGTAAL | PSDEQLKSGTASVVCLLNN |
| | GCLVKDYFPEPVTVSWNSGA | FYPREAKVQWKVDNALQS |
| | LTSGVHTFPAVLQSSGLYSLS | GNSQESVTEQDSKDSTYSLS |
| | SVVTVPSSSLGTQTYICNVNH | STLTLSKADYEKHKVYACE |
| | KPSNTKVDKKVEPKSCDKTH | VTHQGLSSPVTKSFNRGEC |
| | TCPPCPAPELLGGPSVFLFPPK | (SEQ ID NO: 423) |
| | PKDTLMISRTPEVTCVVVDVS | CDR1 (SEQ ID NO: 427)- |
| | HEDPEVKFNWYVDGVEVHN | RASQDISNYLN |
| | AKTKPREEQYNSTYRVVSVL | CDR2 (SEQ ID NO: 428)- |
| | TVLHQDWLNGKEYKCKVSN | TSRLRS |
| | KALPAPIEKTISKAKGQPREP | CDR3 (SEQ ID NO: 429)- |
| | QVYTLPPSREEMTKNQVSLT | QQGHTLPPT |
| | CLVKGFYPSDIAVEWESNGQ | |
| | PENNYKTTPPVLDSDGSFFLY | |
| | SKLTVDKSRWQQGNVFSCSV | |
| | MHEALHNHYTQKSLSLSPGK | |
| | (SEQ ID NO: 422) | |
| | CDR1 (SEQ ID NO: 424)- | |
| | GYTFTDSY | |
| | CDR2 (SEQ ID NO: 425)- | |
| | DNGDS | |
| | CDR3 (SEQ ID NO: 426)- | |
| | VLAPRWYFSV | |
| Anti-TNFRSF4 | QVQLQESGPGLVKPSQTLSLT | DIQMTQSPSSLSASVGDRVT |
| (tavolixizumab) | CAVYGGSFSSGYWNWIRKHP | ITCRASQDISNYLNWYQQK |
| | GKGLEYIGYISYNGITYHNPS | PGKAPKLLIYYTSKLHSGVP |
| | LKSRITINRDTSKNQYSLQLN | SRFSGSGSGTDYTLTISSLQP |
| | SVTPEDTAVYYCARYKYDYD | EDFATYYCQQGSALPWTFG |
| | GGHAMDYWGQGTLVTVSSA | QGTKVEIKRTVAAPSVFIFP |
| | STKGPSVFPLAPSSKSTSGGT | PSDEQLKSGTASVVCLLNN |
| | AALGCLVKDYFPEPVTVSWN | FYPREAKVQWKVDNALQS |
| | SGALTSGVHTFPAVLQSSGLY | GNSQESVTEQDSKDSTYSLS |
| | SLSSVVTVPSSSLGTQTYICNV | STLTLSKADYEKHKVYACE |
| | NHKPSNTKVDKRVEPKSCDK | VTHQGLSSPVTKSFNRGEC |
| | THTCPPCPAPELLGGPSVFLFP | (SEQ ID NO: 431) |
| | PKPKDTLMISRTPEVTCVVVD | CDR1 (SEQ ID NO: 435)- |
| | VSHEDPEVKFNWYVDGVEV | RASQDISNYLN |
| | HNAKTKPREEQYNSTYRVVS | CDR2 (SEQ ID NO: 436)- |
| | VLTVLHQDWLNGKEYKCKV | TSKLH |
| | SNKALPAPIEKTISKAKGQPR | CDR3 (SEQ ID NO: 437)- |
| | EPQVYTLPPSREEMTKNQVSL | QQGSALPWT |
| | TCLVKGFYPSDIAVEWESNG | |
| | QPENNYKTTPPVLDSDGSFFL | |
| | YSKLTVDKSRWQQGNVFSCS | |
| | VMHEALHNHYTQKSLSLSPG | |
| | K (SEQ ID NO: 430) | |
| | CDR1 (SEQ ID NO: 432)- | |
| | GGSFSSGY | |
| | CDR2 (SEQ ID NO: 433)- | |
| | SYNGITYH | |
| | CDR3 (SEQ ID NO: 434)- | |
| | ARYKYDYDGGHAMDY | |
| Anti-TNFRSF8 | QIQLQQSGPEVVKPGASVKIS | DIVLTQSPASLAVSLGQRAT |
| (brentuximab | CKASGYTFTDYYITWVKQKP | ISCKASQSVDFDGDSYMNW |
| vedotin) | GQGLEWIGWIYPGSGNTKYN | YQQKPGQPPKVLIYAASNL |
| | EKFKGKATLTVDTSSSTAFM | ESGIPARFSGSGSGTDFTLNI |
| | QLSSLTSEDTAVYFCANYGN | HPVEEEDAATYYCQQSNED |
| | YWFAYWGQGTQVTVSAAST | PWTFGGGTKLEIKRTVAAP |
| | KGPSVFPLAPSSKSTSGGTAA | SVFIFPPSDEQLKSGTASVV |
| | LGCLVKDYFPEPVTVSWNSG | CLLNNFYPREAKVQWKVD |
| | ALTSGVHTFPAVLQSSGLYSL | NALQSGNSQESVTEQDSKD |
| | SSVVTVPSSSLGTQTYICNVN | STYSLSSTLTLSKADYEKHK |
| | HKPSNTKVDKKVEPKSCDKT | VYACEVTHQGLSSPVTKSF |
| | HTCPPCPAPELLGGPSVFLFPP | NRGEC (SEQ ID NO: 439) |
| | KPKDTLMISRTPEVTCVVVD | CDR1 (SEQ ID NO: 443)- |
| | VSHEDPEVKFNWYVDGVEV | KASQSVDFDGDSYMN |
| | HNAKTKPREEQYNSTYRVVS | CDR2 (SEQ ID NO: 444)- |
| | VLTVLHQDWLNGKEYKCKV | AASNLES |
| | SNKALPAPIEKTISKAKGQPR | CDR3 (SEQ ID NO: 445)- |
| | EPQVYTLPPSRDELTKNQVSL | QQSNEDPWT |
| | TCLVKGFYPSDIAVEWESNG | |
| | QPENNYKTTPPVLDSDGSFFL | |
| | YSKLTVDKSRWQQGNVFSCS | |
| | VMHEALHNHYTQKSLSLSPG | |
| | (SEQ ID NO: 438) | |
| | CDR1 (SEQ ID NO: 440)- | |
| | GYTFTDYY | |
| | CDR2 (SEQ ID NO: 441)- | |
| | YPGSGNT | |
| | CDR3 (SEQ ID NO: 442)- | |
| | ANYGNYWFAY | |
| Anti-TNFRSF8 | QVQLVQSGAEVKKPGASVKV | DIVMTQSPDSLAVSLGERAT |
| (US20100239571A1) | SCKASGYTFTDYYITWVRQA | INCKASQSVDFDGDSYMN |
| | PGQGLEWMGWIYPGSGNTK | WYQQKPGQPPKLLIYAASN |
| | YNEKFKGRVTMTVDTSISTA | LESGVPDRFSGSGSGTDFTL |
| | YMELSRLRSDDTAVYFCANY | TISSLQAEDVAVYYCQQSN |
| | GNYWFAYWGQGTLVTVSS | EDPWTFGQGTKVEIK (SEQ |
| | (SEQ ID NO: 446) | ID NO: 447) |
| | CDR1 (SEQ ID NO: 448)- | CDR1 (SEQ ID NO: 451)- |
| | GYTFTDYY | KASQSVDFDGDSYMN |
| | CDR2 (SEQ ID NO: 449)- | CDR2 (SEQ ID NO: 452)- |
| | YPGSGNT | AASNLES |
| | CDR3 (SEQ ID NO: 450)- | CDR3 (SEQ ID NO: 453)- |
| | ANYGNYWFAY | QQSNEDPWT |
| Anti-TNFRSF9 | QVQLQQWGAGLLKPSETLSL | EIVLTQSPATLSLSPGERATL |
| (urelumab) | TCAVYGGSFSGYYWSWIRQS | SCRASQSVSSYLAWYQQKP |
| | PEKGLEWIGEINHGGYVTYNP | GQAPRLLIYDASNRATGIPA |
| | SLESRVTISVDTSKNQFSLKLS | RFSGSGSGTDFTLTISSLEPE |
| | SVTAADTAVYYCARDYGPG | DFAVYYCQQRSNWPPALTF |
| | NYDWYFDLWGRGTLVTVSS | CGGTKVEIKRTVAAPSVFIF |
| | ASTKGPSVFPLAPCSRSTSEST | PPSDEQLKSGTASVVCLLN |
| | AALGCLVKDYFPEPVTVSWN | NFYPREAKVQWKVDNALQ |
| | SGALTSGVHTFPAVLQSSGLY | SGNSQESVTEQDSKDSTYSL |
| | SLSSVVTVPSSSLGTKTYTCN | SSTLTLSKADYEKHKVYAC |
| | VDHKPSNTKVDKRVESKYGP | EVTHQGLSSPVTKSFNRGEC |
| | PCPPCPAPEFLGGPSVFLFPPK | (SEQ ID NO: 455) |
| | PKDTLMISRTPEVTCVVVDVS | CDR1 (SEQ ID NO: 459)- |
| | QEDPEVQFNWYVDGVEVHN | RASQSVSSYLA |
| | AKTKPREEQFNSTYRVVSVLT | CDR2 (SEQ ID NO: 460)- |
| | VLHQDWLNGKEYKCKVSNK | DASNRATGI |
| | GLPSSIEKTISKAKGQPREPQV | CDR3 (SEQ ID NO: 461)- |
| | YTLPPSQEEMTKNQVSLTCLV | QQRSNWPPALT |
| | KGFYPSDIAVEWESNGQPEN | |
| | NYKTTPPVLDSDGSFFLYSRL | |
| | TVDKSRWQEGNVFSCSVMHE | |
| | ALHNHYTQKSLSLSLGK (SEQ | |
| | ID NO: 454) | |
| | CDR1 (SEQ ID NO: 456)- | |
| | GGSFSGYY | |
| | CDR2 (SEQ ID NO: 457)- | |
| | NHGGYV | |
| | CDR3 (SEQ ID NO: 458)- | |
| | ARDYGPGNYDWYFDL | |
| Anti-TNFRSF9 | EVQLVQSGAEVKKPGESLRIS | SYELTQPPSVSVSPGQTASIT |
| (utomilumab) | CKGSGYSFSTYWISWVRQMP | CSGDNIGDQYAHWYQQKP |
| | GKGLEWMGKIYPGDSYTNYS | GQSPVLVIYQDKNRPSGIPE |
| | PSFQGQVTISADKSISTAYLQ | RFSGSNSGNTATLTISGTQA |
| | WSSLKASDTAMYYCARGYGI | MDEADYYCATYTGFGSLA |
| | FDYWGQGTLVTVSSASTKGP | VFGGGTKLTVLGQPKAAPS |
| | SVFPLAPCSRSTSESTAALGC | VTLFPPSSEELQANKATLVC |
| | LVKDYFPEPVTVSWNSGALT | LISDFYPGAVTVAWKADSS |
| | SGVHTFPAVLQSSGLYSLSSV | PVKAGVETTTPSKQSNNKY |
| | VTVPSSNFGTQTYTCNVDHK | AASSYLSLTPEQWKSHRSY |
| | PSNTKVDKTVERKCCVECPP | SCQVTHEGSTVEKTVAPTE |
| | CPAPPVAGPSVFLFPPKPKDT | CS (SEQ ID NO: 463) |
| | LMISRTPEVTCVVVDVSHEDP | CDR1 (SEQ ID NO: 467)- |
| | EVQFNWYVDGVEVHNAKTK | SGDNIGDQYAH |
| | PREEQFNSTFRVVSVLTVVHQ | CDR2 (SEQ ID NO: 468)- |
| | DWLNGKEYKCKVSNKGLPA | QDKNRPS |
| | PIEKTISKTKGQPREPQVYTLP | CDR3 (SEQ ID NO: 469)- |
| | PSREEMTKNQVSLTCLVKGF | ATYTGFGSLAV |
| | YPSDIAVEWESNGQPENNYK | |
| | TTPPMLDSDGSFFLYSKLTVD | |
| | KSRWQQGNVFSCSVMHEAL | |
| | HNHYTQKSLSLSPGK (SEQ ID | |
| | NO: 462) | |
| | CDR1 (SEQ ID NO: 464)- | |
| | GYSFSTYW | |
| | CDR2 (SEQ ID NO: 465)- | |
| | YPGDSYT | |
| | CDR3 (SEQ ID NO: 466)- | |
| | ARGYGIFDY | |
| Anti-NST5 | EVQLLESGGGLVQPGGSLRLS | QSVLTQPPSASGTPGQRVTI |
| (oleclumab) | CAASGFTFSSYAYSWVRQAP | SCSGSLSNIGRNPVNWYQQ |
| | GKGLEWVSAISGSGGRTYYA | LPGTAPKLLIYLDNLRLSGV |
| | DSVKGRFTISRDNSKNTLYLQ | PDRFSGSKSGTSASLAISGL |
| | MNSLRAEDTAVYYCARLGY | QSEDEADYYCATWDDSHP |
| | GRVDEWGRGTLVTVSSASTK | GWTFGGGTKLTVLGQPKA |
| | GPSVFPLAPSSKSTSGGTAAL | APSVTLFPPSSEELQANKAT |
| | GCLVKDYFPEPVTVSWNSGA | LVCLISDFYPGAVTVAWKA |
| | LTSGVHTFPAVLQSSGLYSLS | DSSPVKAGVETTTPSKQSN |
| | SVVTVPSSSLGTQTYICNVNH | NKYAASSYLSLTPEQWKSH |
| | KPSNTKVDKRVEPKSCDKTH | RSYSCQVTHEGSTVEKTVA |
| | TCPPCPAPEFEGGPSVFLFPPK | PTECS (SEQ ID NO: 471) |
| | PKDTLMISRTPEVTCVVVDVS | CDR1 (SEQ ID NO: 475)- |
| | HEDPEVKFNWYVDGVEVHN | SGSLSNIGRNPVN |
| | AKTKPREEQYNSTYRVVSVL | CDR2 (SEQ ID NO: 476)- |
| | TVLHQDWLNGKEYKCKVSN | LDNLRLS |
| | KALPASIEKTISKAKGQPREP | CDR3 (SEQ ID NO: 477)- |
| | QVYTLPPSREEMTKNQVSLT | ATWDDSHPGWT |
| | CLVKGFYPSDIAVEWESNGQ | |
| | PENNYKTTPPVLDSDGSFFLY | |
| | SKLTVDKSRWQQGNVFSCSV | |
| | MHEALHNHYTQKSLSLSPGK | |
| | (SEQ ID NO: 470) | |
| | CDR1 (SEQ ID NO: 472)- | |
| | GFTFSSYA | |
| | CDR2 (SEQ ID NO: 473)- | |
| | SGSGGRT | |
| | CDR3 (SEQ ID NO: 474)- | |
| | ARLGYGRVDE | |
| Anti-NST5 | QVQLVESGGGVVQPGRSLRL | EIVLTQSPATLSLSPGERATL |
| (US20170253665A1) | SCAASGFTFSNYGMHWVRQ | SCRASQGVSSYLAWYQQKP |
| | APGKGLEWVAVILYDGSNKY | GQAPRLLIYDASNRATGIPA |
| | YPDSVKGRFTISRDNSKNTLY | RFSGSGPGTDFTLTISSLEPE |
| | LQMNSLRAEDTAVYYCARG | DFAVYYCQQRSNWHLTFG |
| | GSSWYPDSFDIWGQGTMVTV | GGTKVEIK (SEQ ID NO: 479) |
| | SS (SEQ ID NO: 478) | CDR1 (SEQ ID NO: 483)- |
| | CDR1 (SEQ ID NO: 480)- | RASQGVSSYLA |
| | NYGMH | CDR2 (SEQ ID NO: 484)- |
| | CDR2 (SEQ ID NO: 481)- | DASNRAT |
| | VILYDGSNKYYPDSVK | CDR3 (SEQ ID NO: 485)- |
| | CDR3 (SEQ ID NO: 482)- | QQRSNWHLT |
| | GGSSWYPDSFDI | |
| Anti-TNFRSF18 | QVQLVESGGGVVQPGRSLRL | DIQMTQSPSSLSASVGDRVT |
| (US20170253665A1) | SCAASGFTFSSYAMHWVRQA | ITCRASQTIYNYLNWYQQK |
| | PGKGLEWVAVISYDGSNKYY | PGKAPKLLIYAASSLQSGVP |
| | ADSVKGRFTISRDNSKNTLYL | SRFGGRGYGTDFTLTINSLQ |
| | QMNSLRAEDTAVYYCARGIA | PEDFATYFCQQSYTSPLTFG |
| | AAGPPYYYYYYYMDVWGK | QGTKVDIK (SEQ ID NO: 487) |
| | GTTVTVSS (SEQ ID NO: 486) | CDR1 (SEQ ID NO: 491)- |
| | CDR1 (SEQ ID NO: 488)- | QTIYNYLN |
| | GFTFSSY | CDR2 (SEQ ID NO: 492)- |
| | CDR2 (SEQ ID NO: 489)- | AASSLQS |
| | SYDGSN | CDR3 (SEQ ID NO: 493)- |
| | CDR3 (SEQ ID NO: 490)- | QQSYTSPLT |
| | GIAAAGPPYYYYYYYMDV | |
| Anti-TNFRSF18 | QVQLVESGGGVVQPGRSLRL | EIVLTQSPGTLSLSPGERATL |
| (US9701751 B2) | SCAASGFTFSSYAMSWVRQA | SCRASESVDXYGVSFMNW |
| | PGKGLEWVASISSGGTTYYPD | YQQKPGQAPRLLIYAASXQ |
| | SVKGRFTISRDNSKNTLYLQM | GSGIPDRFSGSGSGTDFTLTI |
| | NSLRAEDTAVYYCARVGGY | SRLEPEDFAVYYCQQTKEV |
| | YDSMDYWGQGTLVTVSS | TWTFGQGTKVEIKR (SEQ |
| | (SEQ ID NO: 494) | ID NO: 495) |
| | CDR1 (SEQ ID NO: 496)- | CDR1 (SEQ ID NO: 499)- |
| | GFTFSSYA | RASESVDXYGVSFMN |
| | CDR2 (SEQ ID NO: 497)- | CDR2 (SEQ ID NO: 500)- |
| | SSGGTT | AASXQGS |
| | CDR3 (SEQ ID NO: 498)- | CDR3 (SEQ ID NO: 501)- |
| | ARVGGYYDSMDY | QQTKEVTWT |

*References in parenthesis indicate the sources of peptide sequences.

Alternatively, antigen-binding sites that bind to each of T_{reg} associated antigens can be routinely identified by screening for binding to the amino acid sequence of each antigen. For example, antigen-binding sites that bind to CCR8 can be routinely identified by screening for binding to the amino acid sequence of CCR8 is defined by SEQ ID NO:502.

SEQ ID NO: 502
MDYTLDLSVTTVTDYYYPDIFSSPCDAELIQTNGKLLLAVFYCLLFVFSL
LGNSLVILVLVVCKKLRSITDVYLLNLALSDLLFVFSFPFQTYYLLDQWV
FGTVMCKVVSGFYYIGFYSSMFFITLMSVDRYLAVVHAVYALKVRTIRMG
TTLCLAVWLTAIMATIPLLVFYQVASEDGVLQCYSFYNQQTLKWKIFTNF
KMNILGLLIPFTIFMFCYIKILHQLKRCQNHNKTKAIRLVLIVVIASLLF
WVPFNVVLFLTSLHSMHILDGCSISQQLTYATHVTEIISFTHCCVNPVIY
AFVGEKFKKHLSEIFQKSCSQIFNYLGRQMPRESCEKSSSCQQHSSRSSS
VDYILLILRHRRQGKHWTSTQRKADFQHPAGAVGPEPTDRGLQWRSSPAA
DAQEENLYAAVKDTQPEDGVEMDTRAAASEAPQDVTYAQLHSLTLRRKAT
EPPPSQEREPPAEPSIYATLAIH

Antigen-binding sites that bind to CD7 can be routinely identified by screening for binding to the amino acid sequence of CD7 is defined by SEQ ID NO:503.

SEQ ID NO: 503
MAGPPRLLLLPLLLALARGLPGALAAQEVQQSPHCTTVPVGASVNITCST
SGGLRGIYLRQLGPQPQDIIYYEDGVVPTTDRRFRGRIDFSGSQDNLTIT
MHRLQLSDTGTYTCQAITEVNVYGSGTLVLVTEEQSQGWHRCSDAPPRAS
ALPAPPTGSALPDPQTASALPDPPAASALPAALAVISFLLGLGLGVACVL
ARTQIKKLCSWRDKNSAACVVYEDMSHSRCNTLSSPNQYQ

Antigen-binding sites that bind to CTLA4 can be routinely identified by screening for binding to the amino acid sequence of CTLA4 is defined by SEQ ID NO:504.

SEQ ID NO: 504
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

Antigen-binding sites that bind to CX3CR1 can be routinely identified by screening for binding to the amino acid sequence of CX3CR1 is defined by SEQ ID NO:505.

SEQ ID NO: 505
MREPLEAFKLADLDFRKSSLASGWRMASGAFTMDQFPESVTENFEYDDLA
EACYIGDIVVFGTVFLSIFYSVIFAIGLVGNLLVVFALTNSKKPKSVTDI
YLLNLALSDLLFVATLPFWTHYLINEKGLHNAMCKFTTAFFFIGFFGSIF
FITVISIDRYLAIVLAANSMNNRTVQHGVTISLGVWAAAILVAAPQFMFT
KQKENECLGDYPEVLQEIWPVLRNVETNFLGFLLPLLIMSYCYFRIIQTL
FSCKNHKKAKAIKLILLVVIVFFLFWTPYNVMIFLETLKLYDFFPSCDMR
KDLRLALSVTETVAFSHCCLNPLIYAFAGEKFRRYLYHLYGKCLAVLCGR
SVHVDFSSSESQRSRHGSVLSSNFTYHTSDGDALLLL

Antigen-binding sites that bind to ENTPD1 can be routinely identified by screening for binding to the amino acid sequence of LILRB2 is defined by SEQ ID NO:506.

SEQ ID NO: 506
MGREELFLTFSFSSGFQESNVKTFCSKNILAILGFSSIIAVIALLAVGLT
QNKALPENVKYGIVLDAGSSHTSLYIYKWPAEKENDTGVVHQVEECRVKG
PGISKFVQKVNEIGIYLTDCMERAREVIPRSQHQETPVYLGATAGMRLLR
MESEELADRVLDVVERSLSNYPFDFQGARIITGQEEGAYGWITINYLLGK
FSQKTRWFSIVPYETNNQETFGALDLGGASTQVTFVPQNQTIESPDNALQ
FRLYGKDYNVYTHSFLCYGKDQALWQKLAKDIQVASNEILRDPCFHPGYK
KVVNVSDLYKTPCTKRFEMTLPFQQFEIQGIGNYQQCHQSILELFNTSYC
PYSQCAFNGIFLPPLQGDFGAFSAFYFVMKFLNLTSEKVSQEKVTEMMKK
FCAQPWEEIKTSYAGVKEKYLSEYCFSGTYILSLLLQGYHFTADSWEHIH
FIGKIQGSDAGWTLGYMLNLTNMIPAEQPLSTPLSHSTYVFLMVLFSLVL
FTVAIIGLLIFHKPSYFWKDMV

Antigen-binding sites that bind to HAVCR2 can be routinely identified by screening for binding to the amino acid sequence of HAVCR2 is defined by SEQ ID NO:507.

SEQ ID NO: 507
MFSHLPFDCVLLLLLLLLTRSSEVEYRAEVGQNAYLPCFYTPAAPGNLVP
VCWGKGACPVFECGNVVLRTDERDVNYWTSRYWLNGDFRKGDVSLTIENV
TLADSGIYCCRIQIPGIMNDEKFNLKLVIKPAKVTPAPTRQRDFTAAFPR
MLTTRGHGPAETQTLGSLPDINLTQISTLANELRDSRLANDLRDSGATIR
IGIYIGAGICAGLALALIFGALIFKWYSHSKEKIQNLSLISLANLPPSGL
ANAVAEGIRSEENIYTIEENVYEVEEPNEYYCYVSSRQQPSQPLGCRFAM
P

Antigen-binding sites that bind to IL1R2 can be routinely identified by screening for binding to the amino acid sequence of IL1R2 is defined by SEQ ID NO:508.

SEQ ID NO: 508
MLRLYVLVMGVSAFTLQPAAHTGAARSCRFRGRHYKREFRLEGEPVALRC
PQVPYWLWASVSPRINLTWHKNDSARTVPGEEETRMWAQDGALWLLPALQ
EDSGTYVCTTRNASYCDKMSIELRVFENTDAFLPFISYPQILTLSTSGVL
VCPDLSEFTRDKTDVKIQWYKDSLLLDKDNEKFLSVRGTTHLLVHDVALE
DAGYYRCVLTFAHEGQQYNITRSIELRIKKKKEETIPVIISPLKTISASL
GSRLTIPCKVFLGTGTPLTTMLWWTANDTHIESAYPGGRVTEGPRQEYSE
NNENYIEVPLIFDPVTREDLHMDFKCVVHNTLSFQTLRTTVKEASSTFSW
GIVLAPLSLAFLVLGGIWMHRRCKHRTGKADGLTVLWPHHQDFQSYPK

Antigen-binding sites that bind to PDCD1LG2 can be routinely identified by screening for binding to the amino acid sequence of PDCD1LG2 is defined by SEQ ID NO:509.

SEQ ID NO: 509
MIFLLLMLSLELQLHQIAALFTVTVPKELYIIEHGSNVTLECNFDTGSHV
NLGAITASLQKVENDTSPHRERATLLEEQLPLGKASFHIPQVQVRDEGQY
QCIIIYGVAWDYKYLTLKVKASYRKINTHILKVPETDEVELTCQATGYPL
AEVSWPNVSVPANTSHSRTPEGLYQVTSVLRLKPPPGRNFSCVFWNTHVR
ELTLASIDLQSQMEPRTHPTWLLHIFIPFCIIAFIFIATVIALRKQLCQK
LYSSKDTTKRPVTTTKREVNSAI

Antigen-binding sites that bind to TIGIT can be routinely identified by screening for binding to the amino acid sequence of TIGIT is defined by SEQ ID NO:510.

SEQ ID NO: 510
MRWCLLLIWAQGLRQAPLASGMMTGTIETTGNISAEKGGSIILQCHLSST
TAQVTQVNWEQQDQLLAICNADLGWHISPSFKDRVAPGPGLGLTLQSLTV
NDTGEYFCIYHTYPDGTYTGRIFLEVLESSVAEHGARFQIPLLGAMAATL
VVICTAVIVVVALTRKKKALRIHSVEGDLRRKSAGQEEWSPSAPSPPGSC
VQAEAAPAGLCGEQRGEDCAELHDYFNVLSYRSLGNCSFFTETG

Antigen-binding sites that bind to TNFRSF4 can be routinely identified by screening for binding to the amino acid sequence of TNFRSF4 is defined by SEQ ID NO:511.

SEQ ID NO: 511
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

Antigen-binding sites that bind to TNFRSF8 can be routinely identified by screening for binding to the amino acid sequence of TNFRSF8 is defined by SEQ ID NO:512.

SEQ ID NO: 512
MRVLLAALGLLFLGALRAFPQDRPFEDTCHGNPSHYYDKAVRRCCYRCPM
GLFPTQQCPQRPTDCRKQCEPDYYLDEADRCTACVTCSRDDLVEKTPCAW
NSSRVCECRPGMFCSTSAVNSCARCFFHSVCPAGMIVKFPGTAQKNTVCE
PASPGVSPACASPENCKEPSSGTIPQAKPTPVSPATSSASTMPVRGGTRL
AQEAASKLTRAPDSPSSVGRPSSDPGLSPTQPCPEGSGDCRKQCEPDYYL
DEAGRCTACVSCSRDDLVEKTPCAWNSSRTCECRPGMICATSATNSCARC
VPYPICAAETVTKPQDMAEKDTTFEAPPLGTQPDCNPTPENGEAPASTSP
TQSLLVDSQASKTLPIPTSAPVALSSTGKPVLDAGPVLFWVILVLVVVVG
SSAFLLCHRRACRKRIRQKLHLCYPVQTSQPKLELVDSRPRRSSTQLRSG
ASVTEPVAEERGLMSQPLMETCHSVGAAYLESLPLQDASPAGGPSSPRDL
PEPRVSTEHTNNKIEKIYIMKADTVIVGTVKAELPEGRGLAGPAEPELEE
ELEADHTPHYPEQETEPPLGSCSDVMLSVEEEGKEDPLPTAASGK

Antigen-binding sites that bind to TNFRSF9 can be routinely identified by screening for binding to the amino acid sequence of TNFRSF9 is defined by SEQ ID NO:513.

SEQ ID NO: 513
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

Antigen-binding sites that bind to GEM can be routinely identified by screening for binding to the amino acid sequence of GEM is defined by SEQ ID NO:514.

SEQ ID NO: 514
MTLNNVTMRQGTVGMQPQQQRWSIPADGRHLMVQKEPHQYSHRNRHSATP
EDHCRRSWSSDSTDSVISSESGNTYYRVVLIGEQGVGKSTLANIFAGVHD
SMDSDCEVLGEDTYERTLMVDGESATIILLDMWENKGENEWLHDHCMQVG
DAYLIVYSITDRASFEKASELRIQLRRARQTEDIPIILVGNKSDLVRCRE
VSVSEGRACAVVFDCKFIETSAAVQHNVKELFEGIVRQVRLRRDSKEKNE
RRLAYQKRKESMPRKARRFWGKIVAKNNKNMAFKLKSKSCHDLSVL

Antigen-binding sites that bind to NT5E can be routinely identified by screening for binding to the amino acid sequence of NT5E is defined by SEQ ID NO:515.

SEQ ID NO: 515
MCPRAARAPATLLLALGAVLWPAAGAWELTILHTNDVHSRLEQTSEDSSK
CVNASRCMGGVARLFTKVQQIRRAEPNVLLLDAGDQYQGTIWFTVYKGAE
VAHFMNALRYDAMALGNHEFDNGVEGLIEPLLKEAKFPILSANIKAKGPL
ASQISGLYLPYKVLPVGDEVVGIVGYTSKETPFLSNPGTNLVFEDEITAL
QPEVDKLKTLNVNKIIALGHSGFEMDKLIAQKVRGVDVVVGGHSNTFLYT
GNPPSKEVPAGKYPFIVTSDDGRKVPVVQAYAFGKYLGYLKIEFDERGNV
ISSHGNPILLNSSIPEDPSIKADINKWRIKLDNYSTQELGKTIVYLDGSS
QSCRFRECNMGNLICDAMINNNLRHTDEMFWNHVSMCILNGGGIRSPIDE
RNNGTITWENLAAVLPFGGTFDLVQLKGSTLKKAFEHSVHRYGQSTGEFL
QVGGIHVVYDLSRKPGDRVVKLDVLCTKCRVPSYDPLKMDEVYKVILPNF
LANGGDGFQMIKDELLRHDSGDQDINVVSTYISKMKVIYPAVEGRIKFST
GSHCHGSFSLIFLSLWAVIFVLYQ

Antigen-binding sites that bind to TNFRSF18 can be routinely identified by screening for binding to the amino acid sequence of TNFRSF18 is defined by SEQ ID NO:516.

SEQ ID NO: 516
MAQHGAMGAFRALCGLALLCALSLGQRPTGGPGCGPGRLLLGTGTDARCC
RVHTTRCCRDYPGEECCSEWDCMCVQPEFHCGDPCCTTCRHHPCPPGQGV
QSQGKFSFGFQCIDCASGTFSGGHEGHCKPWTDCCWRCRRRPKTPEAASS
PRKSGASDRQRRRGGWETCGCEPGRPPGPPTAASPSPGAPQAAGALRSAL
GRALLPWQQKWVQEGGSDQRPGPCSSAAAAGPCRRERETQSWPPSSLAGP
DGVGS

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, 5354, E356, E357, K360, Q362, S364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the CK of a human IgG1 constant region may be at amino acid E123, F116, 5176, V163, 5174, and/or T164.

Alternatively, amino acid substitutions could be selected from the following sets of substitutions shown in Table 8.

TABLE 8

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 9.

TABLE 9

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 10.

TABLE 10

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 11.

TABLE 11

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, |
| | K392M, K392V, K392F |
| | K392D, K392E, K409F, |
| | K409W, T411D and T411E |

Alternatively, at least one amino acid substitutions could be selected from the following set of substitutions in Table 12, where the position(s) indicated in the First Polypeptide column is replaced by any known negatively-charged amino acid, and the position(s) indicated in the Second Polypeptide Column is replaced by any known positively-charged amino acid.

TABLE 12

| First Polypeptide | Second Polypeptide |
|---|---|
| K392, K370, K409, or K439 | D399, E356, or E357 |

Alternatively, at least one amino acid substitutions could be selected from the following set of in Table 13, where the position(s) indicated in the First Polypeptide column is replaced by any known positively-charged amino acid, and the position(s) indicated in the Second Polypeptide Column is replaced by any known negatively-charged amino acid.

TABLE 13

| First Polypeptide | Second Polypeptide |
|---|---|
| D399, E356, or E357 | K409, K439, K370, or K392 |

Alternatively, amino acid substitutions could be selected from the following set in Table 14.

TABLE 14

| First Polypeptide | Second Polypeptide |
|---|---|
| T350V, L351Y, F405A, and | T350V, T366L, K392L, and T394W |
| Y407V | |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, 5364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, 5364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, 5364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, 5364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, 5400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, 5400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by 0347R, D399V and F405T substitutions.

In some embodiments, the amino acid sequence of one polypeptide chain of the antibody constant region differs from the amino acid sequence of an IgG1 constant region by 0347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

In some embodiments, the amino acid sequence of one polypeptide chain of the antibody constant (human IgG1) region may be SEQ ID NO:164.

SEQ ID NO: 164
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFLLYSKLTVDKSRW
QQGNVFSCSVMHEALHNHYTQKSLSLSPG

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

The multi-specific proteins described herein include an NKG2D-binding site, a CD16-binding site, and a tumor-associated antigen selected from any one of the antigens provided in Table 15. In some embodiments, the multi-specific proteins bind simultaneously to cells expressing NKG2D and/or CD16, such as NK cells, and to tumor cells expressing a tumor-associated antigen selected from any one of the antigens provided in Table 15. Binding of the multi-specific proteins to NK cells can enhance the activity of the NK cells toward destruction of the tumor cells.

TABLE 15

| Type of Antigen | Biological Name |
|---|---|
| Chemokine receptor | CXCR4 |
| Cell surface α chain of the IL-2 | CD25 |
| receptor | |
| Adhesion molecule | Very late antigen-4 (VLA-4) |
| Transmembrane glycoprotein | CD44 |
| Aminopeptidase N | CD13 |
| 3-fucosyl-N-acetyl-lactosamine | CD15 |
| Integrin-associated protein | CD47 |
| Cell surface glycoprotein | CD81 |
| Type II integral membrane protein | CD23 |
| Member of tumor necrosis factor | CD40 |
| receptors (TNFR) | |
| Member of the tumor necrosis factor | CD70 |
| superfamily | |
| Subunit of B-cell antigen receptor | CD79a or CD79b |
| (BCR) | |
| Member of the B7 family of immune | CD80 |
| coregulatory proteins | |
| Type I cytokine receptor | CRLF2 (also known as thymic |
| | stromal lymphopoietin (TSLP) |
| | receptor (TSLPR) |
| Member of the signaling lymphocytic | SLAMF7 (also named CD319) |
| activation molecule (SLAM) family | |
| receptors | |
| Heparin sulphate proteoglycan | CD138 |
| Multifunctional ectoenzyme that | CD38 |
| catalyzes the synthesis and | |
| hydrolysis of cyclic ADP- | |
| ribose (cADPR) from NAD⁺ | |
| to ADP-ribose | |
| T-cell associated tumor antigen | T-cell receptor beta-1 chain C |
| | region (TRBC1) |
| T-cell associated tumor antigen | T-cell receptor beta-2 chain C |
| | region (TRBC2) |
| Leukocyte immunoglobulin-like | LILRB1, LILRB2, LILRB3, |
| receptors (LILR) | LILRB4, LILRB5, LILRA1, |
| | LILRA2, LILRA3, LILRA4, |
| | LILRA5, and LILRA6 |
| Regulatory T cell expressing | CCR8, CD7, CTLA4, CX3CR1, |
| protein | ENTPD1, HAVCR2, IL-1R2, |
| | PDCD1LG2, TIGIT, TNFRSF4, |
| | TNFRSF8, TNFRSF9, GEM, |
| | NT5E, and TNFRSF18 |

In some embodiments, the multi-specific proteins bind to a tumor-associated antigen selected from any one of the antigens provided in Table 15 with a similar affinity to the corresponding monoclonal antibody (i.e., a monoclonal antibody containing the same a tumor-associated antigen-binding site as the one incorporated in the multi-specific proteins (selected from any one of the antigens provided in Table 15)). In some embodiments, the multi-specific proteins are more effective in killing the tumor cells expressing a tumor-associated antigen selected from any one of the antigens provided in Table 15 than the corresponding monoclonal antibodies.

In certain embodiments, the multi-specific proteins described herein, which include an NKG2D-binding site and a binding site for a tumor-associated antigen selected from any one of the antigens provided in Table 15, activate primary human NK cells when co-culturing with cells expressing the tumor-associated antigen. NK cell activation is marked by the increase in CD107a degranulation and IFN-γ cytokine production. Furthermore, compared to a corresponding monoclonal antibody for a tumor-associated antigen selected from any one of the antigens provided in Table 15, the multi-specific proteins may show superior activation of human NK cells in the presence of cells expressing the tumor-associated antigen.

In certain embodiments, the multi-specific proteins described herein, which include an NKG2D-binding site and a binding site for a tumor-associated antigen selected from any one of the antigens provided in Table 15, enhance the activity of rested and IL-2-activated human NK cells co-culturing with cells expressing the tumor-associated antigen.

In certain embodiments, compared to a corresponding monoclonal antibody that binds to a tumor-associated antigen selected from any one of the antigens provided in Table 15, the multi-specific proteins offer an advantage in targeting tumor cells that express medium and low levels of the tumor-associated antigen. The multi-specific binding proteins described herein may be more effective in reducing tumor growth and killing cancer cells. For example, TriNKETs A49-TriNKET-CXCR4-Hz515H7 (an NKG2D-binding domain from clone ADI-27749 and a CXCR4-binding domain derived from Hz515H7), A44-TriNKET-CXCR4-Hz515H7 (an NKG2D-binding domain from clone ADI-27744 and a CXCR4-binding domain derived from Hz515H7), and C26-TriNKET-CXCR4-Hz515H7 (an NKG2D-binding domain from clone ADI-28226 and a CXCR4-binding domain derived from Hz515H7) have enhanced potency and maximum lysis CXCR4-expressing target cells, compared to an anti-CXCR4 monoclonal antibody.

### III. Therapeutic Applications

The invention provides methods for treating cancer using a multi-specific binding protein described herein and/or a pharmaceutical composition described herein. The methods may be used to treat a variety of cancers which express CXCR4 by administering to a patient in need thereof a therapeutically effective amount of a multi-specific binding protein described herein.

The therapeutic method can be characterized according to the cancer to be treated. For example, in certain embodiments, the cancer is acute myeloid leukemia, multiple myeloma, diffuse large B cell lymphoma, thymoma, adenoid cystic carcinoma, gastrointestinal cancer, renal cancer, breast cancer, glioblastoma, lung cancer, ovarian cancer, brain cancer, prostate cancer, pancreatic cancer, or melanoma.

In certain other embodiments, the cancer is a solid tumor. In certain other embodiments, the cancer is colon cancer, bladder cancer, cervical cancer, endometrial cancer, esophageal cancer, leukemia, liver cancer, rectal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VlPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to CXCR4: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

In some other embodiments, when the second binding site binds CXCR4, the cancer to be treated is selected from acute myeloid leukemia, multiple myeloma, diffuse large B cell lymphoma, thymoma, adenoid cystic carcinoma, gastrointestinal cancer, renal cancer, breast cancer, glioblastoma, lung cancer, ovarian cancer, brain cancer, prostate cancer, pancreatic cancer, and melanoma.

In some other embodiments, when the second binding site binds CD25, the cancer to be treated is selected from acute myeloid leukemia, chronic lymphocytic leukemia, glioblastoma, bladder cancer, colon cancer, germ cell tumors, lung cancer, osteosarcoma, melanoma, ovarian cancer, multiple myeloma, head and neck cancer, renal cell cancer, and breast cancer.

In some other embodiments, when the second binding site binds VLA4, CD44, CD13, CD15, CD47, or CD81, the cancer to be treated is selected from acute myeloid leukemia, multiple myeloma, chronic lymphocytic leukemia, B cell lymphoma, T cell lymphoma, Hodgkin lymphoma, breast cancer, glioblastoma, head and neck cancer, ovarian cancer, prostate cancer, melanoma, lung cancer, pancreatic cancer, liver cancer, gastric cancer, thyroid cancer, and brain cancer.

In some other embodiments, when the second binding site binds CD23, CD40, CD70, CD79a, CD79b, CD80, or CRLF2, the cancer to be treated is selected from B cell malignancies, Non-Hodgkin lymphoma, chronic lymphocytic leukemia, acute lymphoblastic leukemia, multiple myeloma, diffuse large B cell lymphoma, follicular lymphoma, T cell lymphoma, renal cancer, glioblastoma, head and neck cancer, nasopharyngeal carcinoma, bladder cancer, cervical cancer, kidney cancer, and ovarian cancer.

In some other embodiments, when the second binding site binds LILRB1, LILRB2, LILRB3, LILRB4, LILRB5, LILRA1, LILRA2, LILRA3, LILRA4, LILRA5, or LILRA6, the cancer to be treated is selected from AML, B cell leukemia, B cell lymphoma, multiple myeloma, T cell leukemia, T cell lymphoma, lung cancer, gastric cancer, breast cancer, and pancreas cancer, wherein the method comprises administering an effective amount of protein according to any one of claims 1-24 or a formulation according to claim 25 to a patient.

### IV. Combination Therapy

Another aspect of the invention provides for combination therapy. A multi-specific binding protein described herein can be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Pharmaceutical compositions can contain a therapeutically effective amount of a multi-specific binding protein comprising an antigen (listed in Table 15) site.

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/mL of disodium phosphate dihydrate, 0.7 to 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e g, mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein of the present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and which are not intended to limit the invention.

### Example 1—NKG2D Binding Domains Bind to NKG2D

### NKG2D-Binding Domains Bind to Purified Recombinant NKG2D

The nucleic acid sequences of human, mouse, or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (comprising heavy chain and light chain variable domains selected from SEQ ID NOs:101-104, or anti-mouse NKG2D clones MI-6 and CX-5 available at eBioscience) was added to each well.

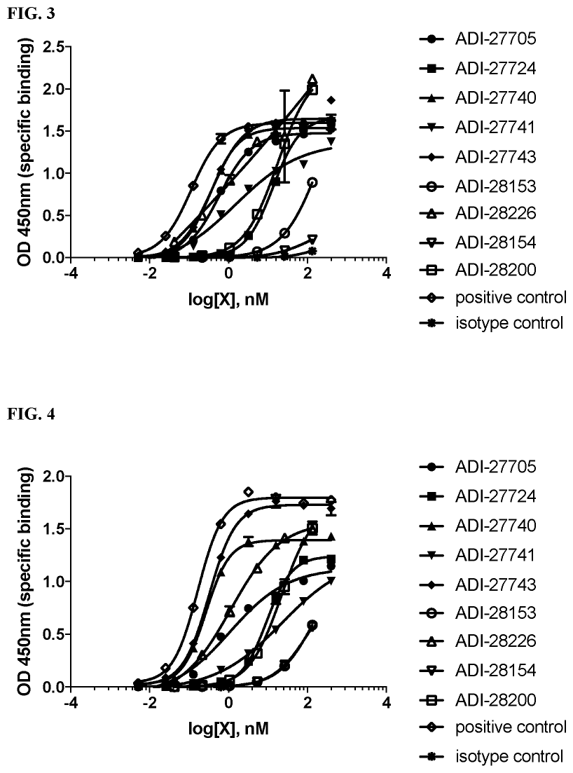

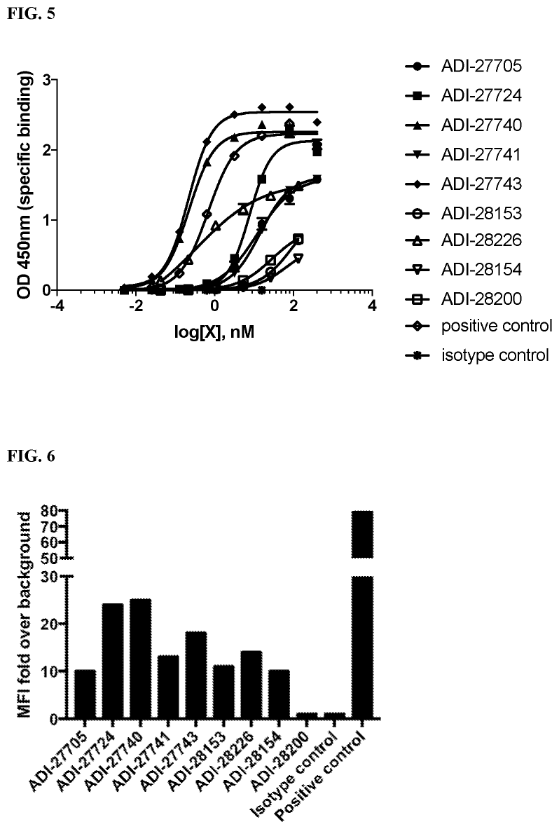

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 3) and cynomolgus (FIG. 4) recombinant NKG2D-Fc with similar affinity, but with lower affinity to mouse (FIG. 5) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control, or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

NKG2D-binding domains produced by all clones bound to EL4 cells expressing human and mouse NKG2D. Positive control antibodies (comprising heavy chain and light chain variable domains selected from SEQ ID NOs:101-104, or anti-mouse NKG2D clones MI-6 and CX-5 available at eBioscience) gave the best FOB binding signal. The NKG2D-binding affinity for each clone was similar between cells expressing human NKG2D (FIG. 6) and mouse (FIG. 7) NKG2D.

### Example 2—NKG2D-Binding Domains Block Natural Ligand Binding to NKG2D

Competition with ULBP-6

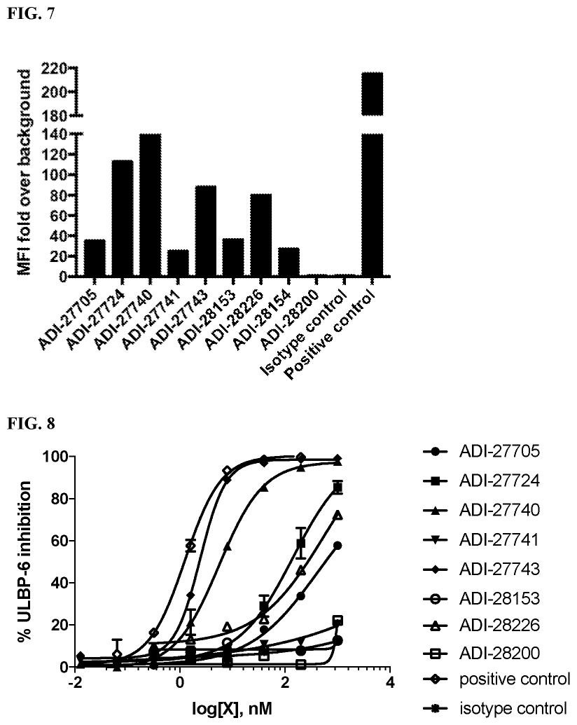

Recombinant human NKG2D-Fc proteins were adsorbed to wells of a microplate, and the wells were blocked with bovine serum albumin to reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (comprising heavy chain and light chain variable domains selected from SEQ ID NOs:101-104) and various NKG2D-binding domains blocked ULBP-6 binding to NKG2D, while isotype control showed little competition with ULBP-6 (FIG. 8).

### ULBP-6 Sequence is Represented by SEQ ID NO:108

(SEQ ID NO: 108)
MAAAAIPALLLCLPLLFLLFGWSRARRDDPHSLCYDITVIPKFRPGPRWC
AVQGQVDEKTFLHYDCGNKTVTPVSPLGKKLNVTMAWKAQNPVLREVVDI
LTEQLLDIQLENYTPKEPLTLQARMSCEQKAEGHSSGSWQFSIDGQTFLL
FDSEKRMWTTVHPGARKMKEKWENDKDVAMSFHYISMGDCIGWLEDFLMG
MDSTLEPSAGAPLAMSSGTTQLRATATTLILCCLLIILPCFILPGI

Competition with MICA

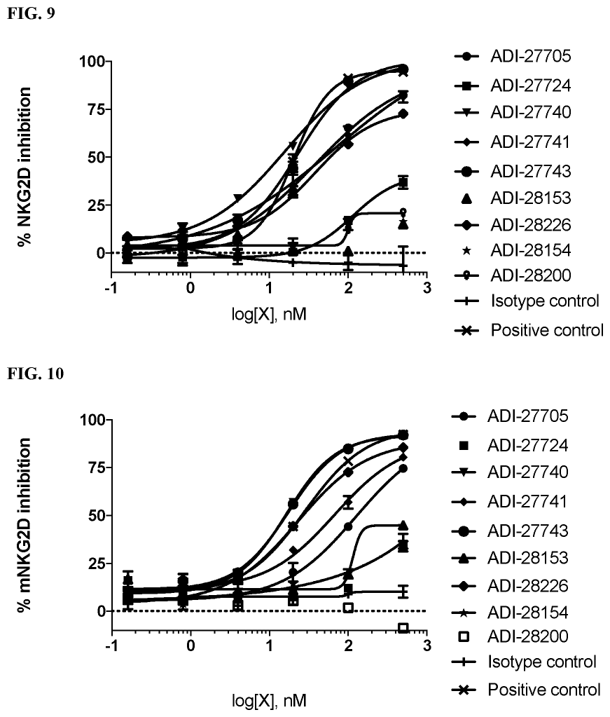

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (comprising heavy chain and light chain variable domains selected from SEQ ID NOs:101-104) and various NKG2D-binding domains blocked MICA binding to NKG2D, while isotype control showed little competition with MICA (FIG. 9).

Competition with Rae-1 Delta

Recombinant mouse Rae-1delta-Fc (purchased from R&D Systems) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (comprising heavy chain and light chain variable domains selected from SEQ ID NOs:101-104, or anti-mouse NKG2D clones MI-6 and CX-5 available at eBioscience) and various NKG2D-binding domain clones blocked Rae-1delta binding to mouse NKG2D, while the isotype control antibody showed little competition with Rae-1delta (FIG. 10).

### Example 3—NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

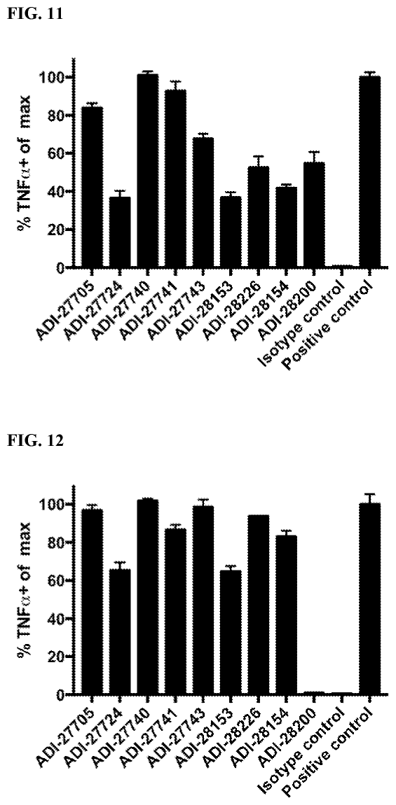

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-α production, an indicator for NKG2D activation, was assayed by flow cytometry. The percentage of TNF-α positive cells was normalized to the cells treated with the positive control. All NKG2D-binding domains activated both human NKG2D (FIG. 11) and mouse NKG2D (FIG. 12).

### Example 4—NKG2D-Binding Domains Activate NK Cells

### Primary Human NK Cells

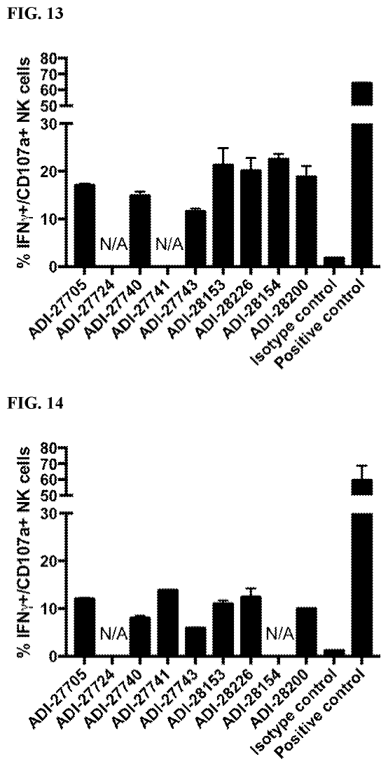

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-γ. CD107a and IFN-γ staining were analyzed in CD3⁻CD56⁺ cells to assess NK cell activation. The increase in CD107a/IFN-γ double-positive cells is indicative of better NK cell activation through engagement of two activating receptors rather than one receptor. NKG2D-binding domains and the positive control (e.g., heavy chain variable domain represent by SEQ ID NO:101 or SEQ ID NO:103, and light chain variable domain represented by SEQ ID NO:102 or SEQ ID NO:104) showed a higher percentage of NK cells becoming CD107a⁺ and IFN-γ⁺ than the isotype control (FIG. 13 & FIG. 14 represent data from two independent experiments, each using a different donor's PBMC for NK cell preparation).

### Primary Mouse NK Cells

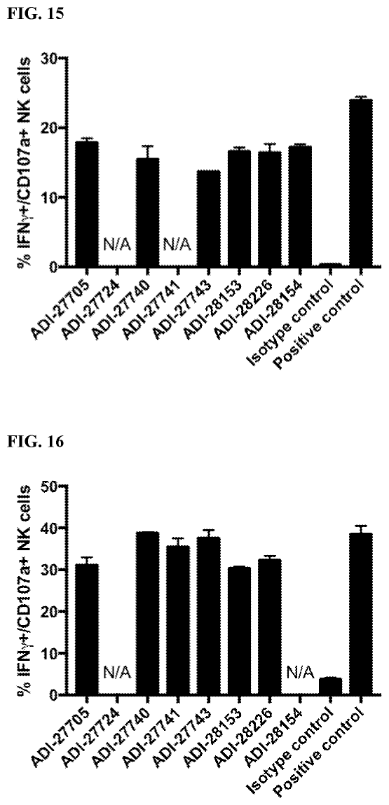

Spleens were obtained from C57Bl/6 mice and crushed through a 70 μm cell strainer to obtain single cell suspension. Cells were pelleted and resuspended in ACK lysis buffer (purchased from Thermo Fisher Scientific # A1049201; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3⁻NK1.1⁺) were then isolated from spleen cells using a negative depletion technique with magnetic beads with typically >90% purity. Purified NK cells were cultured in media containing 100 ng/mL mIL-15 for 48 hours before they were transferred to the wells of a microplate to which the NKG2D-binding domains were adsorbed, and cultured in the media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFN-γ. CD107a and IFN-γ staining were analyzed in CD3⁻NK1.1⁺ cells to assess NK cell activation. The increase in CD107a/IFN-γ double-positive cells is indicative of better NK cell activation through engagement of two activating receptors rather than one receptor. NKG2D-binding domains and the positive control (selected from anti-mouse NKG2D clones MI-6 and CX-5 available at eBioscience) showed a higher percentage of NK cells becoming CD107a⁺ and IFN-γ⁺ than the isotype control (FIG. 15 & FIG. 16 represent data from two independent experiments, each using a different mouse for NK cell preparation).

### Example 5—NKG2D-Binding Domains Enable Cytotoxicity of Target Tumor Cells

Human and mouse primary NK cell activation assays demonstrated increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab fragment regions (NKG2D-binding domain) acted as another targeting arm to activate NK cells. THP-1 cells, which are of human origin and express high levels of Fc receptors, were used as a tumor target and a Perkin Elmer DELFIA Cytotoxicity Kit was used. THP-1 cells were labeled with BATDA reagent, and resuspended at 10⁵/mL in culture media. Labeled THP-1 cells were then combined with NKG2D antibodies and isolated mouse NK cells in wells of a microtiter plate at 37° C. for 3 hours. After incubation, 20 μL of the culture supernatant was removed, mixed with 200 μL of Europium solution and incubated with shaking for 15 minutes in the dark. Fluorescence was measured over time by a PheraStar plate reader equipped with a time-resolved fluorescence module (Excitation 337 nM, Emission 620 nM) and specific lysis was calculated according to the kit instructions.

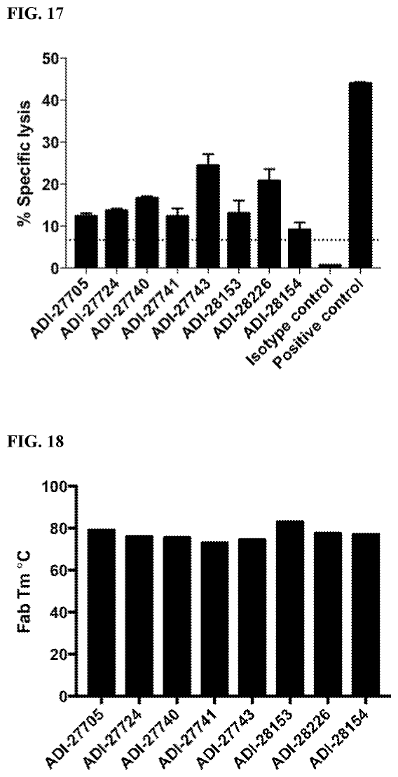

The positive control, ULBP-6—a natural ligand for NKG2D—conjugated to Fc, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 17).

### Example 6—NKG2D Antibodies Show High Thermostability

Melting temperatures of NKG2D-binding domains were assayed using differential scanning fluorimetry. The extrapolated apparent melting temperatures are high relative to typical IgG1 antibodies (FIG. 18).

### Example 7—Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative magnetic beads (StemCell #17955). NK cells were >90% CD3⁻CD56⁺ as determined by flow cytometry. Cells were then expanded 48 hours in media containing 100 ng/mL hIL-2 (Peprotech #200-02) before use in activation assays. Antibodies were coated onto a 96-well flat-bottom plate at a concentration of 2 μg/mL (anti-CD16, Biolegend #302013) and 5 μg/mL (anti-NKG2D, R&D # MAB139) in 100 μL sterile PBS overnight at 4° C. followed by washing the wells thoroughly to remove excess antibody. For the assessment of degranulation IL-2-activated NK cells were resuspended at 5×10⁵ cells/mL in culture media supplemented with 100 ng/mL human IL-2 (hIL2) and 1 μg/mL APC-conjugated anti-CD107a mAb (Biolegend #328619). 1×10⁵ cells/well were then added onto antibody coated plates. The protein transport inhibitors Brefeldin A (BFA, Biolegend #420601) and Monensin (Biolegend #420701) were added at a final dilution of 1:1000 and 1:270, respectively. Plated cells were incubated for 4 hours at 37° C. in 5% CO₂. For intracellular staining of IFN-γ, NK cells were labeled with anti-CD3 (Biolegend #300452) and anti-CD56 mAb (Biolegend #318328), and subsequently fixed, permeabilized and labeled with anti-IFN-γ mAb (Biolegend #506507). NK cells were analyzed for expression of CD107a and IFN-γ by flow cytometry after gating on live CD56⁺CD3⁻cells.

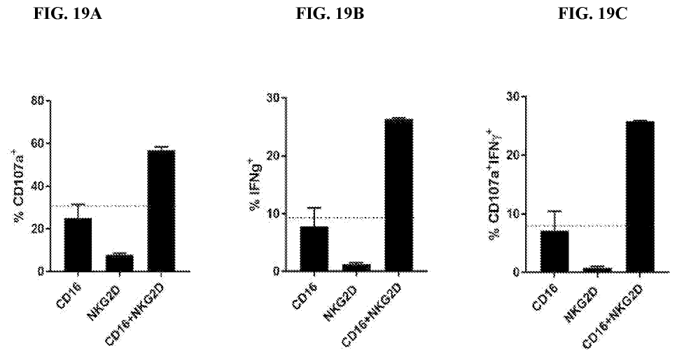

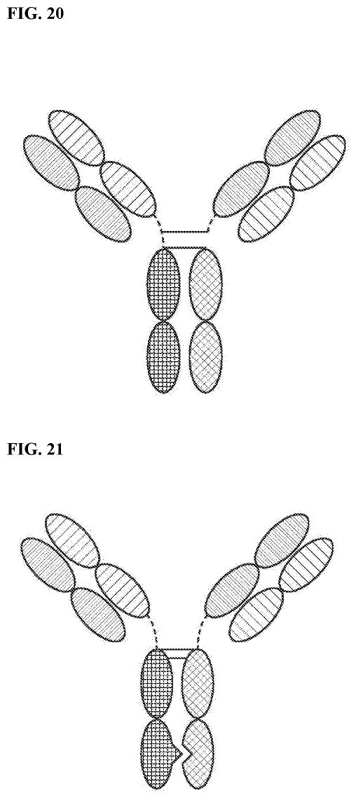

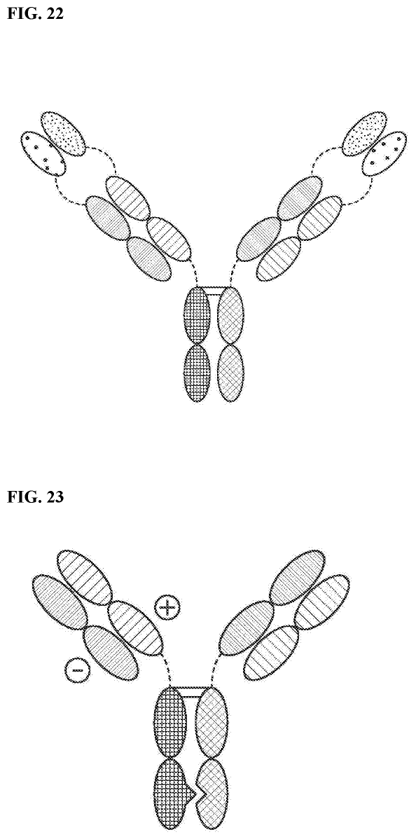

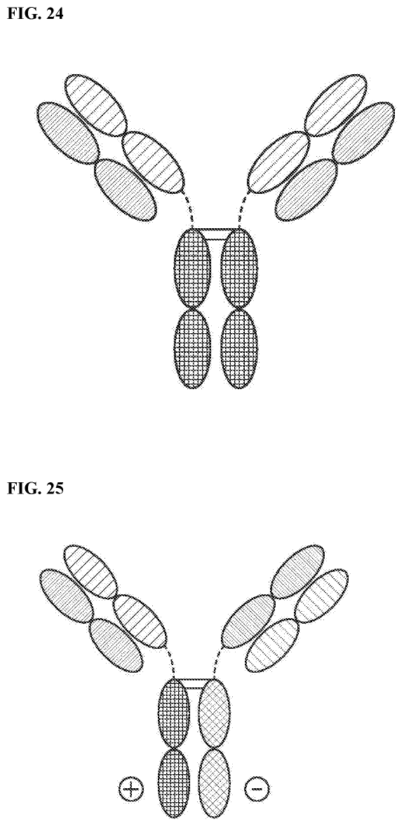

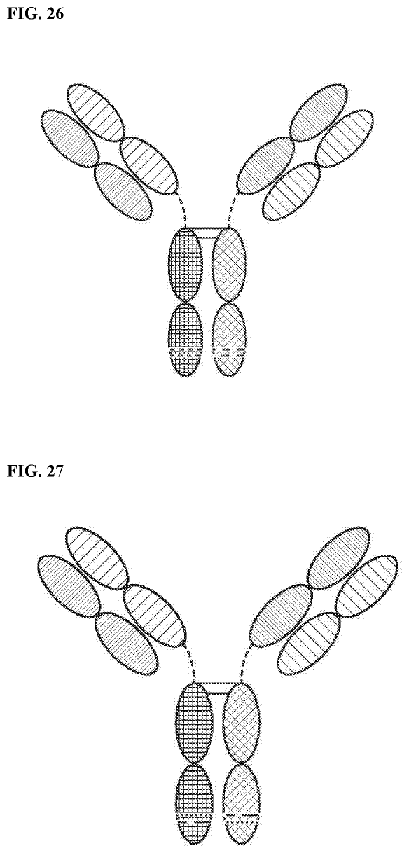

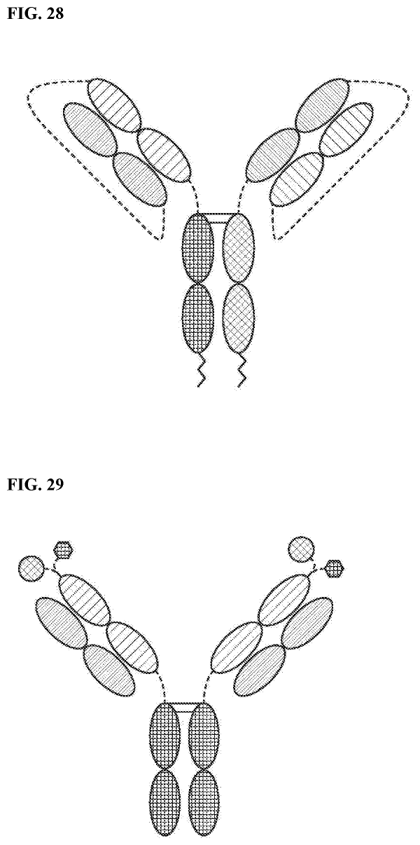

To investigate the relative potency of receptor combination, crosslinking of NKG2D or CD16, and co-crosslinking of both receptors by plate-bound stimulation was performed. As shown in FIG. 19 (FIGS. 19A-19C), combined stimulation of CD16 and NKG2D resulted in highly elevated levels of CD107a (degranulation) (FIG. 19A) and/or IFN-γ production (FIG. 19B). Dotted lines represent an additive effect of individual stimulations of each receptor.

CD107a levels and intracellular IFN-γ production of IL-2-activated NK cells were analyzed after 4 hours of plate-bound stimulation with anti-CD16, anti-NKG2D or a combination of both monoclonal antibodies. Graphs indicate the mean (n=2) ±Sd. FIG. 19A demonstrates levels of CD107a; FIG. 19B demonstrates levels of IFN-γ; FIG. 19C demonstrates levels of CD107a and IFN-γ. Data shown in FIGS. 19A-19C are representative of five independent experiments using five different healthy donors.

### Example 8—Trispecific Binding Protein (TriNKET)-Mediated Enhanced Cytotoxicity of Target Cells

### Expression of CXCR4 on Human Cancer Cell Lines

Human cancer cell lines were screened for surface expression of CXCR4 using flow cytometry. A commercially available antibody against human CXCR4 (clone 12G5) was used for cell staining. Cell lines were harvested from culture, and cells were washed in FACS buffer before staining. Cells were incubated with anti-CXCR4, or corresponding isotype control antibody for 20 minutes on ice. Cells were then washed and resuspended in FACS buffer for analysis. CXCR4 staining was compared to isotype control antibody.

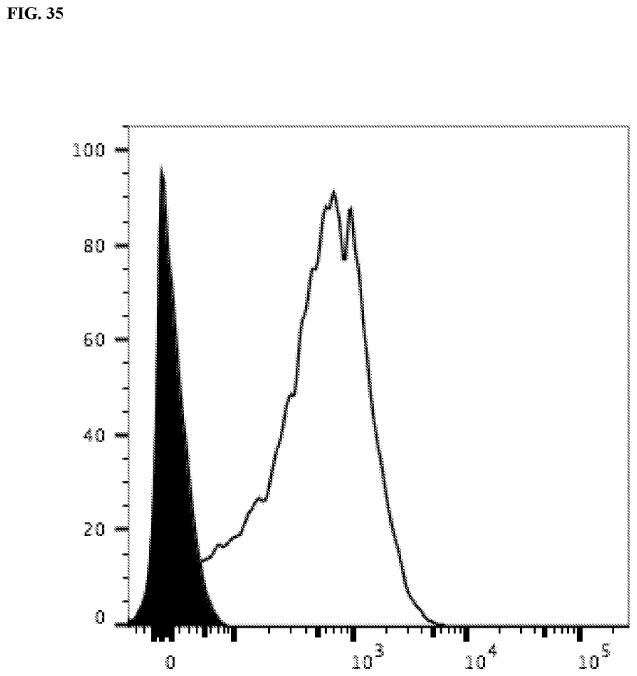

FIG. 35 shows expression of CXCR4 on the surface of Raji human B cell lymphoma cell line. Raji cells demonstrated about a log shift in binding median fluorescent intensity (MFI) when stained with an antibody specific for CXCR4 compared to an isotype control antibody.

### Cytotoxicity Assay

PBMCs were isolated from human peripheral blood buffy coats using density gradient centrifugation. Isolated PBMCs were washed and prepared for NK cell isolation. NK cells were isolated using a negative selection technique with magnetic beads. Purity of isolated NK cells achieved was typically greater than 90% CD3⁻CD56⁺. Isolated NK cells were incubated overnight without cytokine, and used the following day in cytotoxicity assays.

KHYG-1 cells transduced to express CD16-F158V were used to investigate the contribution of dual NKG2D and CD16 stimulation. KHYG-1-CD16V cells were maintained in 10% HI-1-BS-RPMI-1640 with 10 ng/mL IL-2. The day before use as effector cells in killing assays, KHYG-1-CD16V cells were harvested from culture, and cells were washed out of the IL-2 containing media. After washing KHYG-1 cells were resuspended in 10% HI—FBS-RPMI-1640, and were rested overnight without cytokine.

### KHYG-1-CD16V Cytotoxicity Assay

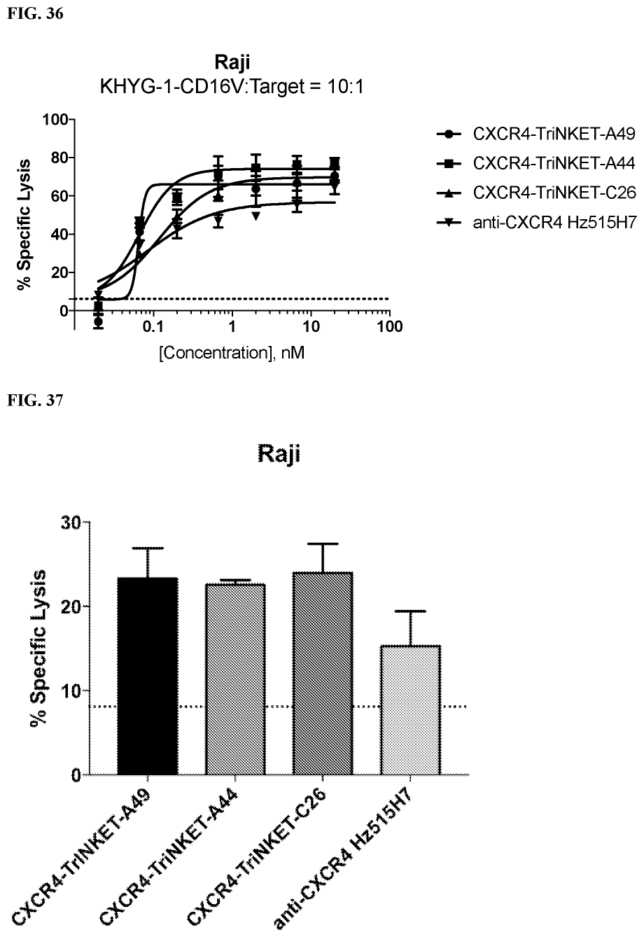

FIG. 36 shows CXCR4-targeted TriNKETs enhance KHYG-1 killing of Raji target cells in a dose-dependent manor. KHYG-1 cells showed weak activity against Raji cells at a 10:1 effector-to-target ratio, with about 6% lysis of target cells. A monoclonal antibody against CXCR4, Hz515H7, was able to enhance KHYG-1 activity. Three TriNKETs using the Hz515H7 CXCR4 binding domain were designed using three different NKG2D binding domains. TriNKETs tested were A49-TriNKET-CXCR4-Hz515H7 (an NKG2D-binding domain from clone ADI-27749 and a CXCR4-binding domain derived from Hz515H7), A44-TriNKET-CXCR4-Hz515H7 (an NKG2D-binding domain from clone ADI-27744 and a CXCR4-binding domain derived from Hz515H7), and C26-TriNKET-CXCR4-Hz515H7 (an NKG2D-binding domain from clone ADI-28226 and a CXCR4-binding domain derived from Hz515H7). All three TriNKETs showed enhanced potency and maximum lysis of Raji target cells compared to the monoclonal antibody.

### DELFIA Cytotoxicity Assay

Human cancer cell lines expressing a target of interest were harvested from culture, washed with HBS, and resuspended in growth media at 10⁶ cells/mL for labeling with BATDA reagent (Perkin Elmer, AD0116). Manufacturer instructions were followed for labeling of the target cells. After labeling, cells were washed 3 times with HBS and resuspended at 0.5×10⁵ cells/mL in culture media. To prepare the background wells, an aliquot of the labeled cells was put aside, and the cells were spun out of the media. 100 μL of the media was carefully added to wells in triplicate to avoid disturbing the pelleted cells. 100 μL of BATDA-labeled cells were added to each well of the 96-well plate. Wells were saved for spontaneous release from target cells and prepared for lysis of target cells by addition of 1% Triton-X. Monoclonal antibodies or TriNKETs against the tumor target of interest were diluted in culture media, and 50 μL of diluted mAb or TriNKET was added to each well. Rested NK cells were harvested from culture, washed, and resuspended at 1.0×10⁵-2.0×10⁶ cell/mL in culture media, depending on the desired effector to target cell ratio. 50 μL of NK cells were added to each well of the plate to provide a total of 200 μL culture volume. The plate was incubated at 37° C. with 5% CO₂ for 2-4 hours before developing the assay.

After culturing for 2-3 hours, the plate was removed from the incubator and the cells were pelleted by centrifugation at 200×g for 5 minutes. 20 μL of culture supernatant was transferred to a clean microplate provided from the manufacturer, and 200 μL of room temperature Europium solution was added to each well. The plate was protected from light and incubated on a plate shaker at 250 rpm for 15 minutes. The plate was read using a SpectraMax® i3X instrument (Molecular Devices), and percent specific lysis was calculated (% Specific lysis=(Experimental release—Spontaneous release)/(Maximum release−Spontaneous release))×100).

### Primary Human NK Cytotoxicity Assay

FIG. 37 shows CXCR4-targeted TriNKETs enhance primary NK cell killing of the CXCR4 positive tumor cell line Raji. Human NK cells showed weak activity against Raji cells at a 5:1 effector-to-target ratio, with 8% lysis of target cells. A monoclonal antibody against CXCR4, Hz515H7, was able to enhance NK cell activity to about 15% lysis. Three TriNKETs using the Hz515H7 CXCR4 binding domain were designed using three different NKG2D binding domains. All three TriNKETs showed enhanced NK cell mediated lysis compared to the monoclonal antibody.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A protein comprising:
(a) a first antigen-binding site that binds NKG2D;
(b) a second antigen-binding site that binds an antigen selected from the group consisting of: CXCR4, CD25, VLA4, CD44, CD13, CD15, CD47, CD81, CD23, CD40, CD70, CD79a, CD79b, CD80, CRLF2, SLAMF7, CD138, CD38, T-cell receptor beta-1 chain C region (TRBC1), T-cell receptor beta-2 chain C region (TRBC2), leukocyte immunoglobulin-like receptor family member selected from LILRB2, LILRB1, LILRB3, LILRB4, LILRB5, LILRA1, LILRA2, LILRA3, LILRA4, LILRA5, and LILRA6, and a protein expressed from regulatory T cells selected from a group consisting of CCR8, CD7, CTLA4, CX3CR1, ENTPD1, HAVCR2, IL-1R2, PDCD1LG2, TIGIT, TNFRSF4, TNFRSF8, TNFRSF9, GEM, NT5E, and TNFRSF18; and
(c) an antibody Fc domain or a portion thereof sufficient to bind CD16, or a third antigen-binding site that binds CD16.

2. A protein comprising:
(a) a first antigen-binding site that binds NKG2D;
(b) a second antigen-binding site that binds CXCR4; and
(c) an antibody Fc domain or a portion thereof sufficient to bind CD16, or a third antigen-binding site that binds CD16.

3. A protein comprising:
(a) a first antigen-binding site that binds NKG2D;
(b) a second antigen-binding site that binds CD25; and
(c) an antibody Fc domain or a portion thereof sufficient to bind CD16, or a third antigen-binding site that binds CD16.

4. A protein comprising:
(a) a first antigen-binding site that binds NKG2D;
(b) a second antigen-binding site that binds a tumor associated antigen selected from VLA4, CD44, CD13, CD15, CD47, and CD81; and
(c) an antibody Fc domain or a portion thereof sufficient to bind CD16, or a third antigen-binding site that binds CD16.

5. A protein comprising:
(a) a first antigen-binding site that binds NKG2D;
(b) a second antigen-binding site that binds a tumor associated antigen selected from CD23, CD40, CD70, CD79a, CD79b, CD80, and CRLF2; and
(c) an antibody Fc domain or a portion thereof sufficient to bind CD16, or a third antigen-binding site that binds CD16.

6. A protein comprising:
(a) a first antigen-binding site that binds NKG2D;
(b) a second antigen-binding site that binds a multiple myeloma associated antigen selected from SLAMF7, CD138 and CD38; and
(c) an antibody Fc domain or a portion thereof sufficient to bind CD16, or a third antigen-binding site that binds CD16.

7. A protein comprising:
(a) a first antigen-binding site that binds NKG2D;
(b) a second antigen-binding site that binds a T-cell associated tumor antigen selected from T-cell receptor beta-1 chain C region (TRBC1) and T-cell receptor beta-2 chain C region (TRBC2); and
(c) an antibody Fc domain or a portion thereof sufficient to bind CD16, or a third antigen-binding site that binds CD16.

8. A protein comprising:
(a) a first antigen-binding site that binds NKG2D;
(b) a second antigen-binding site that binds a leukocyte immunoglobulin-like receptor family member selected from LILRB2, LILRB1, LILRB3, LILRB4, LILRB5, LILRA1, LILRA2, LILRA3, LILRA4, LILRA5, and LILRA6; and
(c) an antibody Fc domain or a portion thereof sufficient to bind CD16, or a third antigen-binding site that binds CD16.

9. A protein comprising:
(a) a first antigen-binding site that binds NKG2D;
(b) a second antigen-binding site that binds a protein expressed from regulatory T cells selected from a group consisting of CCR8, CD7, CTLA4, CX3CR1, ENTPD1, HAVCR2, IL-1R2, PDCD1LG2, TIGIT, TNFRSF4, TNFRSF8, TNFRSF9, GEM, NT5E, and TNFRSF18; and
(c) an antibody Fc domain or a portion thereof sufficient to bind CD16, or a third antigen-binding site that binds CD16.

10. The protein of any one of claims 1-9, wherein the first antigen-binding site binds to NKG2D in humans, non-human primates, and rodents.

11. The protein of claim any one of claims 1-10, wherein the first antigen-binding site comprises a heavy chain variable domain and a light chain variable domain.

12. A protein according to claim 11, wherein the heavy chain variable domain and the light chain variable domain are present on the same polypeptide.

13. A protein according to claim 11 or 12, wherein the second antigen-binding site comprises a heavy chain variable domain and a light chain variable domain.

14. A protein according to claim 13, wherein the heavy chain variable domain and the light chain variable domain of the second antigen-binding site are present on the same polypeptide.

15. A protein according to claim 13 or 14, wherein the light chain variable domain of the first antigen-binding site has an amino acid sequence identical to the amino acid sequence of the light chain variable domain of the second antigen-binding site.

16. A protein according to any one of the preceding claims, wherein the first antigen-binding site comprises a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, and SEQ ID NO:93.

17. A protein according to any one of claims 1-15, wherein the first antigen-binding site comprises a heavy chain variable domain at least 90% identical to SEQ ID NO:41 and a light chain variable domain at least 90% identical to SEQ ID NO:42.

18. A protein according to any one of claims 1-15, wherein the first antigen-binding site comprises a heavy chain variable domain at least 90% identical to SEQ ID NO:49 and a light chain variable domain at least 90% identical to SEQ ID NO:50.

19. A protein according to any one of claims 1-15, wherein the first antigen-binding site comprises a heavy chain variable domain at least 90% identical to SEQ ID NO:57 and a light chain variable domain at least 90% identical to SEQ ID NO:58.

20. A protein according to any one of claims 1-15, wherein the first antigen-binding site comprises a heavy chain variable domain at least 90% identical to SEQ ID NO:59 and a light chain variable domain at least 90% identical to SEQ ID NO:60.

21. A protein according to any one of claims 1-15, wherein the first antigen-binding site comprises a heavy chain variable domain at least 90% identical to SEQ ID NO:61 and a light chain variable domain at least 90% identical to SEQ ID NO:62.

22. A protein according to any one of claims 1-15, wherein the first antigen-binding site comprises a heavy chain variable domain at least 90% identical to SEQ ID NO:69 and a light chain variable domain at least 90% identical to SEQ ID NO:70.

23. A protein according to any one of claims 1-15, wherein the first antigen-binding site comprises a heavy chain variable domain at least 90% identical to SEQ ID NO:77 and a light chain variable domain at least 90% identical to SEQ ID NO:78.

24. A protein according to any one of claims 1-15, wherein the first antigen-binding site comprises a heavy chain variable domain at least 90% identical to SEQ ID NO:85 and a light chain variable domain at least 90% identical to SEQ ID NO:86.

25. A protein according to any one of claims 1-15, wherein the first antigen-binding site comprises a heavy chain variable domain at least 90% identical to SEQ ID NO:93 and a light chain variable domain at least 90% identical to SEQ ID NO:94.

26. A protein according to any one of claims 1-15, wherein the first antigen-binding site comprises a heavy chain variable domain at least 90% identical to SEQ ID NO:101 and a light chain variable domain at least 90% identical to SEQ ID NO:102.

27. A protein according to any one of claims 1-15, wherein the first antigen-binding site comprises a heavy chain variable domain at least 90% identical to SEQ ID NO:103 and a light chain variable domain at least 90% identical to SEQ ID NO:104.

28. The protein of any one of claims 1-10, wherein the first antigen-binding site is a single-domain antibody.

29. The protein of claim 28, wherein the single-domain antibody is a V_{H}H fragment or a V_{NAR} fragment.

30. A protein of any one of claim 1-10 or 28-29, wherein the second antigen-binding site comprises a heavy chain variable domain and a light chain variable domain.

31. A protein of claim 30, wherein the heavy chain variable domain and the light chain variable domain of the second antigen-binding site are present on the same polypeptide.

32. A protein of any of claim 1, 2, or 16-31, wherein the second antigen-binding site binds CXCR4, the heavy chain variable domain of the second antigen-binding site comprises an amino acid sequence at least 90% identical to SEQ ID NO:109 and the light chain variable domain of the second antigen-binding site comprises an amino acid sequence at least 90% identical to SEQ ID NO:110.

33. A protein of claim 32, wherein the heavy chain variable domain of the second antigen-binding site comprises an amino acid sequence including:
a heavy chain CDR1 sequence identical to the amino acid sequence of SEQ ID NO:111;
a heavy chain CDR2 sequence identical to the amino acid sequence of SEQ ID NO:112; and
a heavy chain CDR3 sequence identical to the amino acid sequence of SEQ ID NO:113.

34. A protein of claim 33, wherein the light chain variable domain of the second antigen-binding site comprises an amino acid sequence including:
a light chain CDR1 sequence identical to the amino acid sequence of SEQ ID NO:114;
a light chain CDR2 sequence identical to the amino acid sequence of SEQ ID NO:115;
and a light chain CDR3 sequence identical to the amino acid sequence of SEQ ID NO:116.

35. A protein of any one of claim 1, 2, or 16-31, wherein the second antigen-binding site binds CXCR4, the heavy chain variable domain of the second antigen-binding site comprises an amino acid sequence at least 90% identical to SEQ ID NO:117 and the light chain variable domain of the second antigen-binding site comprises an amino acid sequence at least 90% identical to SEQ ID NO:118.

36. A protein of claim 35, wherein the heavy chain variable domain of the second antigen-binding site comprises an amino acid sequence including:
a heavy chain CDR1 sequence identical to the amino acid sequence of SEQ ID NO:119;
a heavy chain CDR2 sequence identical to the amino acid sequence of SEQ ID NO:120; and
a heavy chain CDR3 sequence identical to the amino acid sequence of SEQ ID NO:121.

37. A protein according to claim 36, wherein the light chain variable domain of the second antigen-binding site comprises an amino acid sequence including:
a light chain CDR1 sequence identical to the amino acid sequence of SEQ ID NO:122;
a light chain CDR2 sequence identical to the amino acid sequence of SEQ ID NO:123; and
a light chain CDR3 sequence identical to the amino acid sequence of SEQ ID NO:124.

38. A protein of any one of claim 1, 2, or 16-31, wherein the second antigen-binding site binds CXCR4, the heavy chain variable domain of the second antigen-binding site comprises an amino acid sequence at least 90% identical to SEQ ID NO:522 and the light chain variable domain of the second antigen-binding site comprises an amino acid sequence at least 90% identical to SEQ ID NO:526.

39. A protein of claim 38, wherein the heavy chain variable domain of the second antigen-binding site comprises an amino acid sequence including:
a heavy chain CDR1 sequence identical to the amino acid sequence of SEQ ID NO:523;
a heavy chain CDR2 sequence identical to the amino acid sequence of SEQ ID NO:524; and
a heavy chain CDR3 sequence identical to the amino acid sequence of SEQ ID NO:525.

40. A protein of claim 39, wherein the light chain variable domain of the second antigen-binding site comprises an amino acid sequence including:
a light chain CDR1 sequence identical to the amino acid sequence of SEQ ID NO:527;
a light chain CDR2 sequence identical to the amino acid sequence of SEQ ID NO:528; and
a light chain CDR3 sequence identical to the amino acid sequence of SEQ ID NO:529.

41. A protein of any one of claim 1, 3, or 16-31, wherein the second antigen-binding site binds CD25, the heavy chain variable domain of the second antigen-binding site comprises an amino acid sequence at least 90% identical to SEQ ID NO:134 and the light chain variable domain of the second antigen-binding site comprises an amino acid sequence at least 90% identical to SEQ ID NO:135.

42. A protein of any one of claim 1, 3, or 16-31, wherein the second antigen-binding site binds CD25, the heavy chain variable domain of the second antigen-binding site comprises an amino acid sequence at least 90% identical to SEQ ID NO:142 and the light chain variable domain of the second antigen-binding site comprises an amino acid sequence at least 90% identical to SEQ ID NO:143.

43. A protein of any one of claim 1, 3, or 16-31, wherein the second antigen-binding site binds CD25, the heavy chain variable domain of the second antigen-binding site comprises an amino acid sequence at least 90% identical to SEQ ID NO:150 and the light chain variable domain of the second antigen-binding site comprises an amino acid sequence at least 90% identical to SEQ ID NO:151.

44. A protein of any one of claim 1, 4, or 16-31, wherein the second antigen-binding site binds VLA4/VCAM-1, the heavy chain variable domain of the second antigen-binding site comprises an amino acid sequence at least 90% identical to SEQ ID NO:166 and the light chain variable domain of the second antigen-binding site comprises an amino acid sequence at least 90% identical to SEQ ID NO:167.

45. A protein of any one of claim 1, 4, or 16-31, wherein the second antigen-binding site binds CD44, the heavy chain variable domain of the second antigen-binding site comprises an amino acid sequence at least 90% identical to SEQ ID NO:174 and the light chain variable domain of the second antigen-binding site comprises an amino acid sequence at least 90% identical to SEQ ID NO:175.

46. A protein of any one of claim 1, 4, or 16-31, wherein the second antigen-binding site binds CD47, the heavy chain variable domain of the second antigen-binding site comprises an amino acid sequence at least 90% identical to SEQ ID NO:182 and the light chain variable domain of the second antigen-binding site comprises an amino acid sequence at least 90% identical to SEQ ID NO:183.

47. A protein of any one of claim 1, 5, or 16-31, wherein the second antigen-binding site binds CD23, the heavy chain variable domain of the second antigen-binding site comprises an amino acid sequence at least 90% identical to SEQ ID NO:197 and the light chain variable domain of the second antigen-binding site comprises an amino acid sequence at least 90% identical to SEQ ID NO:198.

48. A protein of any one of claim 1, 5, or 16-31, wherein the second antigen-binding site binds CD40, the heavy chain variable domain of the second antigen-binding site comprises an amino acid sequence at least 90% identical to SEQ ID NO:205 and the light chain variable domain of the second antigen-binding site comprises an amino acid sequence at least 90% identical to SEQ ID NO:206.

49. A protein of any one of claim 1, 5, or 16-31, wherein the second antigen-binding site binds CD40, the heavy chain variable domain of the second antigen-binding site comprises an amino acid sequence at least 90% identical to SEQ ID NO:213 and the light chain variable domain of the second antigen-binding site comprises an amino acid sequence at least 90% identical to SEQ ID NO:214.

50. A protein of any one of claim 1, 5, or 16-31, wherein the second antigen-binding site binds CD40, the heavy chain variable domain of the second antigen-binding site comprises an amino acid sequence at least 90% identical to SEQ ID NO:221 and the light chain variable domain of the second antigen-binding site comprises an amino acid sequence at least 90% identical to SEQ ID NO:222.

51. A protein of any one of claim 1, 5, or 16-31, wherein the second antigen-binding site binds CD40, the heavy chain variable domain of the second antigen-binding site comprises an amino acid sequence at least 90% identical to SEQ ID NO:229 and the light chain variable domain of the second antigen-binding site comprises an amino acid sequence at least 90% identical to SEQ ID NO:230.

52. A protein of any one of claim 1, 5, or 16-31, wherein the second antigen-binding site binds CD70, the heavy chain variable domain of the second antigen-binding site comprises an amino acid sequence at least 90% identical to SEQ ID NO:237 and the light chain variable domain of the second antigen-binding site comprises an amino acid sequence at least 90% identical to SEQ ID NO:238.

53. A protein of any one of claim 1, 5, or 16-31, wherein the second antigen-binding site binds CD79b, the heavy chain variable domain of the second antigen-binding site comprises an amino acid sequence at least 90% identical to SEQ ID NO:245 and the light chain variable domain of the second antigen-binding site comprises an amino acid sequence at least 90% identical to SEQ ID NO:246.

54. A protein of any one of claim 1, 5, or 16-31, wherein the second antigen-binding site binds CD80, the heavy chain variable domain of the second antigen-binding site comprises an amino acid sequence at least 90% identical to SEQ ID NO:253 and the light chain variable domain of the second antigen-binding site comprises an amino acid sequence at least 90% identical to SEQ ID NO:254.

55. A protein of any one of claim 1, 5, or 16-31, wherein the second antigen-binding site binds CRLF2, the heavy chain variable domain of the second antigen-binding site comprises an amino acid sequence at least 90% identical to SEQ ID NO:261 and the light chain variable domain of the second antigen-binding site comprises an amino acid sequence at least 90% identical to SEQ ID NO:262.

56. A protein of any one of claim 1, 6, or 16-31, wherein the second antigen-binding site binds SLAMF7, the heavy chain variable domain of the second antigen-binding site comprises an amino acid sequence at least 90% identical to SEQ ID NO:272 and the light chain variable domain of the second antigen-binding site comprises an amino acid sequence at least 90% identical to SEQ ID NO:273.

57. A protein of any one of claim 1, 6, or 16-31, wherein the second antigen-binding site binds SLAMF7, the heavy chain variable domain of the second antigen-binding site comprises an amino acid sequence at least 90% identical to SEQ ID NO:280 and the light chain variable domain of the second antigen-binding site comprises an amino acid sequence at least 90% identical to SEQ ID NO:281.

58. A protein of any one of claim 1, 6, or 16-31, wherein the second antigen-binding site binds CD138, the heavy chain variable domain of the second antigen-binding site comprises an amino acid sequence at least 90% identical to SEQ ID NO:288 and the light chain variable domain of the second antigen-binding site comprises an amino acid sequence at least 90% identical to SEQ ID NO:289.

59. A protein of any one of claim 1, 6, or 16-31, wherein the second antigen-binding site binds CD38, the heavy chain variable domain of the second antigen-binding site comprises an amino acid sequence at least 90% identical to SEQ ID NO:296 and the light chain variable domain of the second antigen-binding site comprises an amino acid sequence at least 90% identical to SEQ ID NO:297.

60. A protein of any one of claim 1, 6, or 16-31, wherein the second antigen-binding site binds CD38, the heavy chain variable domain of the second antigen-binding site comprises an amino acid sequence at least 90% identical to SEQ ID NO:304 and the light chain variable domain of the second antigen-binding site comprises an amino acid sequence at least 90% identical to SEQ ID NO:305.

61. A protein of any one of claim 1, 9, or 16-31, wherein the second antigen-binding site binds CD7, the heavy chain variable domain of the second antigen-binding site comprises an amino acid sequence at least 90% identical to SEQ ID NO:325 or SEQ ID NO:329.

62. A protein of any one of claim 1, 9, or 16-31, wherein the second antigen-binding site binds CTLA4, the heavy chain variable domain of the second antigen-binding site comprises an amino acid sequence at least 90% identical to SEQ ID NO:333 and the light chain variable domain of the second antigen-binding site comprises an amino acid sequence at least 90% identical to SEQ ID NO:334.

63. A protein of any one of claim 1, 9, or 16-31, wherein the second antigen-binding site binds CTLA4, the heavy chain variable domain of the second antigen-binding site comprises an amino acid sequence at least 90% identical to SEQ ID NO:341 and the light chain variable domain of the second antigen-binding site comprises an amino acid sequence at least 90% identical to SEQ ID NO:342.

64. A protein of any one of claim 1, 9, or 16-31, wherein the second antigen-binding site binds CX3CR1, the heavy chain variable domain of the second antigen-binding site comprises an amino acid sequence at least 90% identical to SEQ ID NO:349 or SEQ ID NO:353.

65. A protein of any one of claim 1, 9, or 16-31, wherein the second antigen-binding site binds ENTPD1, the heavy chain variable domain of the second antigen-binding site comprises an amino acid sequence at least 90% identical to SEQ ID NO:358 and the light chain variable domain of the second antigen-binding site comprises an amino acid sequence at least 90% identical to SEQ ID NO:359.

66. A protein of any one of claim 1, 9, or 16-31, wherein the second antigen-binding site binds ENTPD1, the heavy chain variable domain of the second antigen-binding site comprises an amino acid sequence at least 90% identical to SEQ ID NO:366 and the light chain variable domain of the second antigen-binding site comprises an amino acid sequence at least 90% identical to SEQ ID NO:367.

67. A protein of any one of claim 1, 9, or 16-31, wherein the second antigen-binding site binds HAVCR2, the heavy chain variable domain of the second antigen-binding site comprises an amino acid sequence at least 90% identical to SEQ ID NO:374 and the light chain variable domain of the second antigen-binding site comprises an amino acid sequence at least 90% identical to SEQ ID NO:375.

68. A protein of any one of claim 1, 9, or 16-31, wherein the second antigen-binding site binds HAVCR2, the heavy chain variable domain of the second antigen-binding site comprises an amino acid sequence at least 90% identical to SEQ ID NO:382 and the light chain variable domain of the second antigen-binding site comprises an amino acid sequence at least 90% identical to SEQ ID NO:383.

69. A protein of any one of claim 1, 9, or 16-31, wherein the second antigen-binding site binds PDCDILG2, the heavy chain variable domain of the second antigen-binding site comprises an amino acid sequence at least 90% identical to SEQ ID NO:390 and the light chain variable domain of the second antigen-binding site comprises an amino acid sequence at least 90% identical to SEQ ID NO:391.

70. A protein of any one of claim 1, 9, or 16-31, wherein the second antigen-binding site binds PDCDILG2, the heavy chain variable domain of the second antigen-binding site comprises an amino acid sequence at least 90% identical to SEQ ID NO:398 and the light chain variable domain of the second antigen-binding site comprises an amino acid sequence at least 90% identical to SEQ ID NO:399.

71. A protein of any one of claim 1, 9, or 16-31, wherein the second antigen-binding site binds TIGIT, the heavy chain variable domain of the second antigen-binding site comprises an amino acid sequence at least 90% identical to SEQ ID NO:406 and the light chain variable domain of the second antigen-binding site comprises an amino acid sequence at least 90% identical to SEQ ID NO:407.

72. A protein of any one of claim 1, 9, or 16-31, wherein the second antigen-binding site binds TIGIT, the heavy chain variable domain of the second antigen-binding site comprises an amino acid sequence at least 90% identical to SEQ ID NO:414 and the light chain variable domain of the second antigen-binding site comprises an amino acid sequence at least 90% identical to SEQ ID NO:415.

73. A protein of any one of claim 1, 9, or 16-31, wherein the second antigen-binding site binds TNFRSF4, the heavy chain variable domain of the second antigen-binding site comprises an amino acid sequence at least 90% identical to SEQ ID NO:422 and the light chain variable domain of the second antigen-binding site comprises an amino acid sequence at least 90% identical to SEQ ID NO:423.

74. A protein of any one of claim 1, 9, or 16-31, wherein the second antigen-binding site binds TNFRSF4, the heavy chain variable domain of the second antigen-binding site comprises an amino acid sequence at least 90% identical to SEQ ID NO:430 and the light chain variable domain of the second antigen-binding site comprises an amino acid sequence at least 90% identical to SEQ ID NO:431.

75. A protein of any one of claim 1, 9, or 16-31, wherein the second antigen-binding site binds TNFRSF8, the heavy chain variable domain of the second antigen-binding site comprises an amino acid sequence at least 90% identical to SEQ ID NO:438 and the light chain variable domain of the second antigen-binding site comprises an amino acid sequence at least 90% identical to SEQ ID NO:439.

76. A protein of any one of claim 1, 9, or 16-31, wherein the second antigen-binding site binds TNFRSF8, the heavy chain variable domain of the second antigen-binding site comprises an amino acid sequence at least 90% identical to SEQ ID NO:446 and the light chain variable domain of the second antigen-binding site comprises an amino acid sequence at least 90% identical to SEQ ID NO:447.

77. A protein of any one of claim 1, 9, or 16-31, wherein the second antigen-binding site binds TNFRSF9, the heavy chain variable domain of the second antigen-binding site comprises an amino acid sequence at least 90% identical to SEQ ID NO:454 and the light chain variable domain of the second antigen-binding site comprises an amino acid sequence at least 90% identical to SEQ ID NO:455.

78. A protein of any one of claim 1, 9, or 16-31, wherein the second antigen-binding site binds TNFRSF9, the heavy chain variable domain of the second antigen-binding site comprises an amino acid sequence at least 90% identical to SEQ ID NO:462 and the light chain variable domain of the second antigen-binding site comprises an amino acid sequence at least 90% identical to SEQ ID NO:463.

79. A protein of any one of claim 1, 9, or 16-31, wherein the second antigen-binding site binds NST5, the heavy chain variable domain of the second antigen-binding site comprises an amino acid sequence at least 90% identical to SEQ ID NO:470 and the light chain variable domain of the second antigen-binding site comprises an amino acid sequence at least 90% identical to SEQ ID NO:471.

80. A protein of any one of claim 1, 9, or 16-31, wherein the second antigen-binding site binds NST5, the heavy chain variable domain of the second antigen-binding site comprises an amino acid sequence at least 90% identical to SEQ ID NO:478 and the light chain variable domain of the second antigen-binding site comprises an amino acid sequence at least 90% identical to SEQ ID NO:479.

81. A protein of any one of claim 1, 9, or 16-31, wherein the second antigen-binding site binds TNFRSF18, the heavy chain variable domain of the second antigen-binding site comprises an amino acid sequence at least 90% identical to SEQ ID NO:486 and the light chain variable domain of the second antigen-binding site comprises an amino acid sequence at least 90% identical to SEQ ID NO:487.

82. A protein of any one of claim 1, 9, or 16-31, wherein the second antigen-binding site binds TNFRSF18, the heavy chain variable domain of the second antigen-binding site comprises an amino acid sequence at least 90% identical to SEQ ID NO:494 and the light chain variable domain of the second antigen-binding site comprises an amino acid sequence at least 90% identical to SEQ ID NO:495.

83. A protein of any one of claim 1-12 or 16-29, wherein the second antigen-binding site is a single-domain antibody.

84. The protein of claim 83, wherein the second antigen-binding site is a V_{H}H fragment or a V_{NAR} fragment.

85. A protein according to any one of claims 1-84, wherein the antibody Fc domain comprises a hinge and a CH2 domain.

86. A protein according to any one of claims 1-84, wherein the antibody Fc domain comprises hinge and CH2 domains of a human IgG1 antibody.

87. A protein of claim 85 or 86, wherein the Fc domain comprises an amino acid sequence at least 90% identical to amino acids 234-332 of a human IgG1 antibody.

88. A protein of claim 87, wherein the Fc domain comprises amino acid sequence at least 90% identical to the Fc domain of human IgG1 and differs at one or more positions selected from the group consisting of Q347, Y349, L351, 5354, E356, E357, K360, Q362, 5364, T366, L368, K370, N390, K392, T394, D399, 5400, D401, F405, Y407, K409, T411, K439.

89. A formulation comprising a protein according to any one of the preceding claims and a pharmaceutically acceptable carrier.

90. A cell comprising one or more nucleic acids expressing a protein according to any one of claims 1-88.

91. A method of directly and/or indirectly enhancing tumor cell death, the method comprising exposing a tumor and natural killer cells to a protein according to any one of claims 1-88.

92. A method of treating cancer, wherein the method comprises administering a protein according to any one of claims 1-88 or a formulation according to claim 89 to a patient.

93. The method of claim 92, wherein when the second binding site binds CXCR4, the cancer is selected from the group consisting of acute myeloid leukemia, multiple myeloma, diffuse large B cell lymphoma, thymoma, adenoid cystic carcinoma, gastrointestinal cancer, renal cancer, breast cancer, glioblastoma, lung cancer, ovarian cancer, brain cancer, prostate cancer, pancreatic cancer, and melanoma.

94. The method of claim 92, wherein when the second binding site binds CD25, the cancer is selected from the group consisting of acute myeloid leukemia, chronic lymphocytic leukemia, glioblastoma, bladder cancer, colon cancer, germ cell tumors, lung cancer, osteosarcoma, melanoma, ovarian cancer, multiple myeloma, head and neck cancer, renal cell cancer, and breast cancer.

95. The method of claim 92, wherein, when the second binding site binds VLA4, CD44, CD13, CD15, CD47, or CD81, the cancer is selected from the group consisting of acute myeloid leukemia, multiple myeloma, chronic lymphocytic leukemia, B cell lymphoma, T cell lymphoma, Hodgkin lymphoma, breast cancer, glioblastoma, head and neck cancer, ovarian cancer, prostate cancer, melanoma, lung cancer, pancreatic cancer, liver cancer, gastric cancer, thyroid cancer, and brain cancer.

96. The method of claim 92, wherein when the second binding site binds CD23, CD40, CD70, CD79a, CD79b, CD80, or CRLF2, the cancer is selected from the group consisting of a B cell malignancies, Non-Hodgkin lymphoma, chronic lymphocytic leukemia, acute lymphoblastic leukemia, multiple myeloma, diffuse large B cell lymphoma, follicular lymphoma, T cell lymphoma, renal cancer, glioblastoma, head and neck cancer, nasopharyngeal carcinoma, bladder cancer, cervical cancer, kidney cancer, and ovarian cancer.

97. The method of claim 92, wherein when the second binding site binds LILRB1, LILRB2, LILRB3, LILRB4, LILRB5, LILRA1, LILRA2, LILRA3, LILRA4, LILRA5, or LILRA6, the cancer is selected from the group consisting of AML, B cell leukemia, B cell lymphoma, multiple myeloma, T cell leukemia, T cell lymphoma, lung cancer, gastric cancer, breast cancer, and pancreas cancer.
